SEQUENCE LISTING (61 sequences)

SEQ ID NO: 1 - DNA, 4017 bp, synthetic construct
  source 1..4017: mol_type = other DNA
agggaggggagggatggggggaaagcaagctggaggacaggtgagacagcaggacaggtgaggcgggccctgaggggggggcgggtgggagccaggtgaatgtacggctcttggcggccgagggggggcgggcggcaggaggaggcagagggcggcggaggaggagccccccagcagcgagcggcgagcaactgaccgcggccttctgaccaggaccggagcagggccccaagcccccgggcctggtgggggacgcgcttcttcccacactgtgagcctcagcagctccagccagcggacccgacggctgagaggagccccagaaccaggactgggggatttggagctgggcagagacttaacccccacagcaccgggaagcagccaactcccctcgcctccttcccccttcgtggcttgcggtctctcttccccgcctcggcccccaggaagtgaagatgttacagcctggtcctcatcctccctcaccccaagctgctgctcctggagaagcctggccaggcccctctcaggctccctggcagagcctagaggagaagatggggagccccgaggatgacctgattgggattccattcccggaccacagcagtgagctcctgagctgcctcaatgagcagcgccagctgggccacctatgtgacctcaccatccggacgcagggccttgaataccgcacccacagggctgtgctagctgcctgtagccactacttcaagaagcttttcactgagggcggtggcggagctgtcatgggggccgggggtagcgggacggccactgggggagcaggggccggtgtgtgtgagctggactttgtagggccagaggcactaggcgccctccttgaatttgcctatacagccacactgaccaccagcagcgccaacatgccagctgtgctccaggctgcccgcctgctggagatcccgtgtgtcatcgctgcttgcatggagattctgcagggcagtgggctagaagctcccagcccggacgaggatgactgtgagcgagcccgccagtatctggaggcctttgccacagccacggcctctggagttcccaatggtgaagacagtcctccacaggtgcccctcccaccacctccgccaccgccacctcggcctgttgcccgccgcagccgcaagccccggaaagctttcctgcaaaccaagggggccagagcaaaccacctagtccctgaggtgcccacagtgcccgcccatcccttgacctatgaggaggaggaggtggcgggcagagtgggcagcagtgggggcagtgggccgggggacagctacagccctcccacaggaactgcctcccctcctgagggtccccagagctacgaaccctatgagggtgaggaagaagaagaggagctggtatatcccccagcctatgggctggcgcagggtggcgggcccccgctgtccccagaggagctgggctcagatgaggatgccatcgatcctgacctgatggcctacctaagctccctgcaccaggacaacctggcaccaggcctggacagccaagacaagctggtgcgcaaacgccgctcccagatgcctcaggagtgccctgtctgccacaagatcatccatggggcaggcaaactgcctcgccacatgaggacccacacaggcgagaagccctttgcctgcgaggtctgcggtgttcgattcaccaggaacgacaagctgaagatccacatgcggaagcacacgggagagcgcccctactcatgcccgcactgcccagcccgcttcctgcacagctacgacctcaagaaccacatgcacctgcacacaggggaccggccctatgagtgccacctgtgccacaaggctttcgccaaggaggaccacctgcagcgccacctcaaaggccagaactgcctggaggtgcgcacccgacggcgccgcaaggacgatgcaccaccccactacccaccaccctctaccgctgctgcatcccccgctggcctcgacctctccaatggccacctggacaccttccgcctctctctagctcgattctgggagcagtcagcccccactgggcccccggtctctaccccagggccccctgatgacgatgaggaggaaggggcacccaccacaccccaggctgaaggtgccatggagtcctcttaaagagggacgagggccagactgaagcagcacaaggccggggacacccatgccaagcagtgggagcacgcaggacagacacagcaggggtctggggcacggagccttgctggcatcagcatcagcccttcctcccagagccctcattccaattccaagctaagaaggtattggggcagaggctccccaaattggggtgatcccccaaggagtgatacatatattgtgtatatatttacagctgtattgtaaaagtggggtccctgtccccagctgctcctggggagtagaagcaataatgtatttctaatttgtgggtcccacttcggctatgcgggtttctagggggtgggggcttgggaccaaagccttgccccgcccctatgccccttgggggttttggctgtgtaagggggtgaaggactgcccctccctttcgagacccctccttcctggtttctgttcctttttcctggcagtgaattatgcaaagggggccggcaaaggaagggtaggtgggggaaagccaggtggaagcttgaaagactgggggactgggcctgtaaggaaggagccatcccagtccccctccgccctgctcccggcgctgagtcatggggtcgtggagaagggggcggggtggcctgattggctcgcctgcccctgggggcagtagaggggccccgcccagctaggggagccgctccgttccactcccctccctagccctccctccccacggccctgggcagggaatgtcttgttcccgccgctccctccccggggccagagggcagggcgggccgggcggcgtcctaccctcttctcctcctccccatctcctccccgcccaggtgcgagccggagccgccgccaccgctgccgcccctgactcacgccgcccccgggctggcgcagcgaagggtgtgggacagggtaaggggttggaagagccttgtggagagcgggcgagccggcgccatctggcggccatgctctgagtgggcgagcgccccccgcggccactggagcgagctgtcttcacgctcctcatccaccccagctggtgagcggcgcccccttgccaaggcagtgggcacagaacttctcgcttggccgcaggggaaggggctgcggacctgtgggaaagtgatccccttcccagatccttgccagccgggcttcctgtcaggcaggggagaataatccccactctgctcttaggattgaatccacccccattctgtacatagcctcttctgttggtcttgttgaaatctagtttcagatttttaactacccaattctgctgggggtgggggacacccccccttcctcgctgggtgctggaccccttttgcagcctgggctctgccttgcactatttccccttcctggcctgacggctcctccccctccttaaaaggggcaggttcaggggcccggtgctcttcctcccttccatgcacccccatgcccatttgcacagctgcccaggtacccctaacagtggggaggggtcacagggagggggtagcgggaccagtccctgttatctatttaaaaagtgatgatgtaatatattggggtggcggggagatcgggttgtcctgggcctcatcttagcatttcaggtgatggggggagcccagggctggggagacctggggcccagccccagaaagtggggacaatgtggcctcccttctccctactttcggctttcccagtcagtgccttagggggagaggcactccccccctcctattcccttccccccaccccaactcccccacctcgggtgtaagcgacaggaagaaataataataatttaagattc

SEQ ID NO: 2 - DNA, 3606 bp, synthetic construct
  source 1..3606: mol_type = other DNA
agcagcgagcggcgagcaactgaccgcggccttctgaccaggaccggagcagggccccaagcccccgggcctggtgggggacgcgcttcttcccacactgtgagcctcagcagctccagccagcggacccgacggctgagaggagaagatggggagccccgaggatgacctgattgggattccattcccggaccacagcagtgagctcctgagctgcctcaatgagcagcgccagctgggccacctatgtgacctcaccatccggacgcagggccttgaataccgcacccacagggctgtgctagctgcctgtagccactacttcaagaagcttttcactgagggcggtggcggagctgtcatgggggccgggggtagcgggacggccactgggggagcaggggccggtgtgtgtgagctggactttgtagggccagaggcactaggcgccctccttgaatttgcctatacagccacactgaccaccagcagcgccaacatgccagctgtgctccaggctgcccgcctgctggagatcccgtgtgtcatcgctgcttgcatggagattctgcagggcagtgggctagaagctcccagcccggacgaggatgactgtgagcgagcccgccagtatctggaggcctttgccacagccacggcctctggagttcccaatggtgaagacagtcctccacaggtgcccctcccaccacctccgccaccgccacctcggcctgttgcccgccgcagccgcaagccccggaaagctttcctgcaaaccaagggggccagagcaaaccacctagtccctgaggtgcccacagtgcccgcccatcccttgacctatgaggaggaggaggtggcgggcagagtgggcagcagtgggggcagtgggccgggggacagctacagccctcccacaggaactgcctcccctcctgagggtccccagagctacgaaccctatgagggtgaggaagaagaagaggagctggtatatcccccagcctatgggctggcgcagggtggcgggcccccgctgtccccagaggagctgggctcagatgaggatgccatcgatcctgacctgatggcctacctaagctccctgcaccaggacaacctggcaccaggcctggacagccaagacaagctggtgcgcaaacgccgctcccagatgcctcaggagtgccctgtctgccacaagatcatccatggggcaggcaaactgcctcgccacatgaggacccacacaggcgagaagccctttgcctgcgaggtctgcggtgttcgattcaccaggaacgacaagctgaagatccacatgcggaagcacacgggagagcgcccctactcatgcccgcactgcccagcccgcttcctgcacagctacgacctcaagaaccacatgcacctgcacacaggggaccggccctatgagtgccacctgtgccacaaggctttcgccaaggaggaccacctgcagcgccacctcaaaggccagaactgcctggaggtgcgcacccgacggcgccgcaaggacgatgcaccaccccactacccaccaccctctaccgctgctgcatcccccgctggcctcgacctctccaatggccacctggacaccttccgcctctctctagctcgattctgggagcagtcagcccccactgggcccccggtctctaccccagggccccctgatgacgatgaggaggaaggggcacccaccacaccccaggctgaaggtgccatggagtcctcttaaagagggacgagggccagactgaagcagcacaaggccggggacacccatgccaagcagtgggagcacgcaggacagacacagcaggggtctggggcacggagccttgctggcatcagcatcagcccttcctcccagagccctcattccaattccaagctaagaaggtattggggcagaggctccccaaattggggtgatcccccaaggagtgatacatatattgtgtatatatttacagctgtattgtaaaagtggggtccctgtccccagctgctcctggggagtagaagcaataatgtatttctaatttgtgggtcccacttcggctatgcgggtttctagggggtgggggcttgggaccaaagccttgccccgcccctatgccccttgggggttttggctgtgtaagggggtgaaggactgcccctccctttcgagacccctccttcctggtttctgttcctttttcctggcagtgaattatgcaaagggggccggcaaaggaagggtaggtgggggaaagccaggtggaagcttgaaagactgggggactgggcctgtaaggaaggagccatcccagtccccctccgccctgctcccggcgctgagtcatggggtcgtggagaagggggcggggtggcctgattggctcgcctgcccctgggggcagtagaggggccccgcccagctaggggagccgctccgttccactcccctccctagccctccctccccacggccctgggcagggaatgtcttgttcccgccgctccctccccggggccagagggcagggcgggccgggcggcgtcctaccctcttctcctcctccccatctcctccccgcccaggtgcgagccggagccgccgccaccgctgccgcccctgactcacgccgcccccgggctggcgcagcgaagggtgtgggacagggtaaggggttggaagagccttgtggagagcgggcgagccggcgccatctggcggccatgctctgagtgggcgagcgccccccgcggccactggagcgagctgtcttcacgctcctcatccaccccagctggtgagcggcgcccccttgccaaggcagtgggcacagaacttctcgcttggccgcaggggaaggggctgcggacctgtgggaaagtgatccccttcccagatccttgccagccgggcttcctgtcaggcaggggagaataatccccactctgctcttaggattgaatccacccccattctgtacatagcctcttctgttggtcttgttgaaatctagtttcagatttttaactacccaattctgctgggggtgggggacacccccccttcctcgctgggtgctggaccccttttgcagcctgggctctgccttgcactatttccccttcctggcctgacggctcctccccctccttaaaaggggcaggttcaggggcccggtgctcttcctcccttccatgcacccccatgcccatttgcacagctgcccaggtacccctaacagtggggaggggtcacagggagggggtagcgggaccagtccctgttatctatttaaaaagtgatgatgtaatatattggggtggcggggagatcgggttgtcctgggcctcatcttagcatttcaggtgatggggggagcccagggctggggagacctggggcccagccccagaaagtggggacaatgtggcctcccttctccctactttcggctttcccagtcagtgccttagggggagaggcactccccccctcctattcccttccccccaccccaactcccccacctcgggtgtaagcgacaggaagaaataataataatttaagattca

SEQ ID NO: 3 - DNA, 3594 bp, synthetic construct
  source 1..3594: mol_type = other DNA
agctggaggacaggtgagacagcaggacaggaccggagcagggccccaagcccccgggcctggtgggggacgcgcttcttcccacactgtgagcctcagcagctccagccagcggacccgacggctgagaggagaagatggggagccccgaggatgacctgattgggattccattcccggaccacagcagtgagctcctgagctgcctcaatgagcagcgccagctgggccacctatgtgacctcaccatccggacgcagggccttgaataccgcacccacagggctgtgctagctgcctgtagccactacttcaagaagcttttcactgagggcggtggcggagctgtcatgggggccgggggtagcgggacggccactgggggagcaggggccggtgtgtgtgagctggactttgtagggccagaggcactaggcgccctccttgaatttgcctatacagccacactgaccaccagcagcgccaacatgccagctgtgctccaggctgcccgcctgctggagatcccgtgtgtcatcgctgcttgcatggagattctgcagggcagtgggctagaagctcccagcccggacgaggatgactgtgagcgagcccgccagtatctggaggcctttgccacagccacggcctctggagttcccaatggtgaagacagtcctccacaggtgcccctcccaccacctccgccaccgccacctcggcctgttgcccgccgcagccgcaagccccggaaagctttcctgcaaaccaagggggccagagcaaaccacctagtccctgaggtgcccacagtgcccgcccatcccttgacctatgaggaggaggaggtggcgggcagagtgggcagcagtgggggcagtgggccgggggacagctacagccctcccacaggaactgcctcccctcctgagggtccccagagctacgaaccctatgagggtgaggaagaagaagaggagctggtatatcccccagcctatgggctggcgcagggtggcgggcccccgctgtccccagaggagctgggctcagatgaggatgccatcgatcctgacctgatggcctacctaagctccctgcaccaggacaacctggcaccaggcctggacagccaagacaagctggtgcgcaaacgccgctcccagatgcctcaggagtgccctgtctgccacaagatcatccatggggcaggcaaactgcctcgccacatgaggacccacacaggcgagaagccctttgcctgcgaggtctgcggtgttcgattcaccaggaacgacaagctgaagatccacatgcggaagcacacgggagagcgcccctactcatgcccgcactgcccagcccgcttcctgcacagctacgacctcaagaaccacatgcacctgcacacaggggaccggccctatgagtgccacctgtgccacaaggctttcgccaaggaggaccacctgcagcgccacctcaaaggccagaactgcctggaggtgcgcacccgacggcgccgcaaggacgatgcaccaccccactacccaccaccctctaccgctgctgcatcccccgctggcctcgacctctccaatggccacctggacaccttccgcctctctctagctcgattctgggagcagtcagcccccactgggcccccggtctctaccccagggccccctgatgacgatgaggaggaaggggcacccaccacaccccaggctgaaggtgccatggagtcctcttaaagagggacgagggccagactgaagcagcacaaggccggggacacccatgccaagcagtgggagcacgcaggacagacacagcaggggtctggggcacggagccttgctggcatcagcatcagcccttcctcccagagccctcattccaattccaagctaagaaggtattggggcagaggctccccaaattggggtgatcccccaaggagtgatacatatattgtgtatatatttacagctgtattgtaaaagtggggtccctgtccccagctgctcctggggagtagaagcaataatgtatttctaatttgtgggtcccacttcggctatgcgggtttctagggggtgggggcttgggaccaaagccttgccccgcccctatgccccttgggggttttggctgtgtaagggggtgaaggactgcccctccctttcgagacccctccttcctggtttctgttcctttttcctggcagtgaattatgcaaagggggccggcaaaggaagggtaggtgggggaaagccaggtggaagcttgaaagactgggggactgggcctgtaaggaaggagccatcccagtccccctccgccctgctcccggcgctgagtcatggggtcgtggagaagggggcggggtggcctgattggctcgcctgcccctgggggcagtagaggggccccgcccagctaggggagccgctccgttccactcccctccctagccctccctccccacggccctgggcagggaatgtcttgttcccgccgctccctccccggggccagagggcagggcgggccgggcggcgtcctaccctcttctcctcctccccatctcctccccgcccaggtgcgagccggagccgccgccaccgctgccgcccctgactcacgccgcccccgggctggcgcagcgaagggtgtgggacagggtaaggggttggaagagccttgtggagagcgggcgagccggcgccatctggcggccatgctctgagtgggcgagcgccccccgcggccactggagcgagctgtcttcacgctcctcatccaccccagctggtgagcggcgcccccttgccaaggcagtgggcacagaacttctcgcttggccgcaggggaaggggctgcggacctgtgggaaagtgatccccttcccagatccttgccagccgggcttcctgtcaggcaggggagaataatccccactctgctcttaggattgaatccacccccattctgtacatagcctcttctgttggtcttgttgaaatctagtttcagatttttaactacccaattctgctgggggtgggggacacccccccttcctcgctgggtgctggaccccttttgcagcctgggctctgccttgcactatttccccttcctggcctgacggctcctccccctccttaaaaggggcaggttcaggggcccggtgctcttcctcccttccatgcacccccatgcccatttgcacagctgcccaggtacccctaacagtggggaggggtcacagggagggggtagcgggaccagtccctgttatctatttaaaaagtgatgatgtaatatattggggtggcggggagatcgggttgtcctgggcctcatcttagcatttcaggtgatggggggagcccagggctggggagacctggggcccagccccagaaagtggggacaatgtggcctcccttctccctactttcggctttcccagtcagtgccttagggggagaggcactccccccctcctattcccttccccccaccccaactcccccacctcgggtgtaagcgacaggaagaaataataataatttaagattc

SEQ ID NO: 4 - DNA, 20 bp, synthetic construct
  source 1..20: mol_type = other DNA
cttgcagcactgcctaggct

SEQ ID NO: 5 - DNA, 20 bp, synthetic construct
  source 1..20: mol_type = other DNA
tggttcttgcagcactgcct

SEQ ID NO: 6 - DNA, 20 bp, synthetic construct
  source 1..20: mol_type = other DNA
tcttgcagcactgcctaggc

SEQ ID NO: 7 - DNA, 20 bp, synthetic construct
  source 1..20: mol_type = other DNA
gcccaccttgaagatctgta

SEQ ID NO: 8 - DNA, 20 bp, synthetic construct
  source 1..20: mol_type = other DNA
tgcccaccttgaagatctgt

SEQ ID NO: 9 - DNA, 20 bp, synthetic construct
  source 1..20: mol_type = other DNA
caccttgaagatctgtaggg

SEQ ID NO: 10 - DNA, 20 bp, synthetic construct
  source 1..20: mol_type = other DNA
ttatcatgatactatgtccc

SEQ ID NO: 11 - DNA, 20 bp, synthetic construct
  source 1..20: mol_type = other DNA
agttactgagcacagtgcct

SEQ ID NO: 12 - DNA, 20 bp, synthetic construct
  source 1..20: mol_type = other DNA
gagttactgagcacagtgcc

SEQ ID NO: 13 - DNA, 20 bp, synthetic construct
  source 1..20: mol_type = other DNA
attaatgcctttaaataggt

SEQ ID NO: 14 - DNA, 20 bp, synthetic construct
  source 1..20: mol_type = other DNA
tcttaaacctacctatttaa

SEQ ID NO: 15 - DNA, 20 bp, synthetic construct
  source 1..20: mol_type = other DNA
gctaattaatgcctttaaat

SEQ ID NO: 16 - DNA, 20 bp, synthetic construct
  source 1..20: mol_type = other DNA
taggctgggctcaaactcca

SEQ ID NO: 17 - DNA, 19 bp, synthetic construct
  source 1..19: mol_type = other DNA
ctaggctgggctcaaactc

SEQ ID NO: 18 - DNA, 20 bp, synthetic construct
  source 1..20: mol_type = other DNA
tgagaggatcacttgagccc

SEQ ID NO: 19 - DNA, 20 bp, synthetic construct
  source 1..20: mol_type = other DNA
ctggagtttgagcccagcct

SEQ ID NO: 20 - DNA, 20 bp, synthetic construct
  source 1..20: mol_type = other DNA
ttctttgtttgtttagagac

SEQ ID NO: 21 - DNA, 21 bp, synthetic construct
  source 1..21: mol_type = other DNA
agcccagcctaggcagtgctg

SEQ ID NO: 22 - DNA, 21 bp, synthetic construct
  source 1..21: mol_type = other DNA
gagactggttcttgcagcact

SEQ ID NO: 23 - DNA, 21 bp, synthetic construct
  source 1..21: mol_type = other DNA
tttagagactggttcttgcag

SEQ ID NO: 24 - DNA, 21 bp, synthetic construct
  source 1..21: mol_type = other DNA
tttgtttagagactggttctt

SEQ ID NO: 25 - DNA, 21 bp, synthetic construct
  source 1..21: mol_type = other DNA
tttgtttgtttagagactggt

SEQ ID NO: 26 - DNA, 20 bp, synthetic construct
  source 1..20: mol_type = other DNA
gggccaagaggctcagtcac

SEQ ID NO: 27 - DNA, 20 bp, synthetic construct
  source 1..20: mol_type = other DNA
ggataaagagatcaggtcca

SEQ ID NO: 28 - DNA, 20 bp, synthetic construct
  source 1..20: mol_type = other DNA
tccctacagatcttcaaggt

SEQ ID NO: 29 - DNA, 20 bp, synthetic construct
  source 1..20: mol_type = other DNA
ttcaaggtgggcaaggaact

SEQ ID NO: 30 - DNA, 20 bp, synthetic construct
  source 1..20: mol_type = other DNA
tccctccctacagatcttca

SEQ ID NO: 31 - DNA, 20 bp, synthetic construct
  source 1..20: mol_type = other DNA
ctccctacagatcttcaagg

SEQ ID NO: 32 - DNA, 20 bp, synthetic construct
  source 1..20: mol_type = other DNA
accttgaagatctgtaggga

SEQ ID NO: 33 - DNA, 20 bp, synthetic construct
  source 1..20: mol_type = other DNA
cttcaaggtgggcaaggaac

SEQ ID NO: 34 - DNA, 20 bp, synthetic construct
  source 1..20: mol_type = other DNA
acagatcttcaaggtgggca

SEQ ID NO: 35 - DNA, 20 bp, synthetic construct
  source 1..20: mol_type = other DNA
aagagatcaggtccaaggcc

SEQ ID NO: 36 - DNA, 20 bp, synthetic construct
  source 1..20: mol_type = other DNA
gggcaaggaactgggccaag

SEQ ID NO: 37 - DNA, 20 bp, synthetic construct
  source 1..20: mol_type = other DNA
tagggagggataaagagatc

SEQ ID NO: 38 - DNA, 20 bp, synthetic construct
  source 1..20: mol_type = other DNA
atcaggtccaaggccaggtg

SEQ ID NO: 39 - DNA, 20 bp, synthetic construct
  source 1..20: mol_type = other DNA
aggtccaaggccaggtgcgg

SEQ ID NO: 40 - DNA, 20 bp, synthetic construct
  source 1..20: mol_type = other DNA
tgaaccaccgcacctggcct

SEQ ID NO: 41 - DNA, 21 bp, synthetic construct
  source 1..21: mol_type = other DNA
tccctccctacagatcttcaa

SEQ ID NO: 42 - DNA, 21 bp, synthetic construct
  source 1..21: mol_type = other DNA
caagcatttatcatgatacta

SEQ ID NO: 43 - DNA, 21 bp, synthetic construct
  source 1..21: mol_type = other DNA
tcatgatactatgtcccaggc

SEQ ID NO: 44 - DNA, 20 bp, synthetic construct
  source 1..20: mol_type = other DNA
ccacttccctcctccattaa

SEQ ID NO: 45 - DNA, 20 bp, synthetic construct
  source 1..20: mol_type = other DNA
ctttaatggaggagggaagt

SEQ ID NO: 46 - DNA, 20 bp, synthetic construct
  source 1..20: mol_type = other DNA
cctttaatggaggagggaag

SEQ ID NO: 47 - DNA, 20 bp, synthetic construct
  source 1..20: mol_type = other DNA
tttaatggaggagggaagtg

SEQ ID NO: 48 - DNA, 21 bp, synthetic construct
  source 1..21: mol_type = other DNA
atggaggagggaagtggggtg

SEQ ID NO: 49 - DNA, 21 bp, synthetic construct
  source 1..21: mol_type = other DNA
agagaatctatgtcaccccac

SEQ ID NO: 50 - DNA, 21 bp, synthetic construct
  source 1..21: mol_type = other DNA
aaggcattaattagcgtttgc

SEQ ID NO: 51 - DNA, 21 bp, synthetic construct
  source 1..21: mol_type = other DNA
aataggtaggtttaagagaat

SEQ ID NO: 52 - DNA, 21 bp, synthetic construct
  source 1..21: mol_type = other DNA
catgcactactttaaaatttt

SEQ ID NO: 53 - DNA, 21 bp, synthetic construct
  source 1..21: mol_type = other DNA
aagtagtgcatgcaaacgcta

SEQ ID NO: 54 - DNA, 800 bp, synthetic construct
  source 1..800: mol_type = other DNA
ttctcatttagcaagagtggaaaactgttgtggatcccaggagaagtctggagctaggtagtaggggctagataatagagggctttgactaataggagtttgggccttatcctgtggaaatctgaagggttttgggaaaatgaattcagggctaggtgcaagacagaagggcaaataccagactaagacacacagccactaagagcctctttttttttttttgtccagtcagaggtaatgaggcctatatatttattaggaaggatgattaaccatggctgtagcttcacctaagaggaacgtggctcttgggccgggtgcggtggctcacgcctgtaatcccagcactttgggaggtagaggtgggcggatcacctgaggtcaggagttcaagaccagcctgaccaacatggagaaacctcttctctactaaaaatacaaaattagccgggcgtggtggtgcatgcctgtaatccctgttactcgggaggctgaggcaggagaatcgcttgaacctgggaggtggaggttgcggtgagctgagatcacaccattgcactccagcctggacaacaagagcaaaactctatctcaaaaaaaaagagaggaatgtggctcttgaagaatctttctagtctcatcttccctgtccccttagacacaccttcaaagatcctttttcttttggtttggcttgtctttccccctcactttacctaatccagcccaattccatagccactgctggttcctttaatggaggagggaagtggggtgacatagattctcttaaacctacc

SEQ ID NO: 55 - DNA, 800 bp, synthetic construct
  source 1..800: mol_type = other DNA
tatttaaaggcattaattagcgtttgcatgcactactttaaaattttcctctcaattgcttgagtccaggagttcgaggttacggtgaactatgatttcaccattgcagtccagctgaggcaacagagagagaccctcttaaagaaaaaaaaaactttttcctctcagactctgtccctagcattgactcaccccatcattttttttttttttcttgagatggagtttcaagaaaaaactccaggctggaggcagtggtatggtctcggctcactgcaacctccgcctcccgggttcaagcgattctccctgcctcagcctcccaaatagctgggattacaggcacccgccaccacacccagctaattttttgtatttttagcagagacggggcttctccatattagccaagctggtctggaactcctgacttcaagtgatccacccaccttggcctcccaaagtactgggattacaggcacgagccaccgcgcccggccttcacctattatatctattcttgtcttttaaatagtgagtgacttcacagctcagtctctttgttctgaacttccattctgagtcgcaacccaggtacaccttaggaagggcgggatcccgttatcctgtccctgaacatcaactactgagacagctgatgaagctggctcggtgtgtgacggaggccccctcagagtcagcagagccgagcgtacagcgcacatcaggcagagcagaccagaggcagggaagacacactggggcagggccagtgggcacatctggaccacctgcgaggg

SEQ ID NO: 56 - DNA, 800 bp, synthetic construct
  source 1..800: mol_type = other DNA
ttattcttcacagaggacagctcccaactctttacctctcttcctaatgcaacactcactctcactctcagcagccttataatatctgtaaatgtagaaactatcagttatgaacttctgtaactcccttttcatctttggcacctcaaaactttaatgtatgtaaaagcatgggctttgaaattggtaaactggagctctatcaatctctggctgtgtcaacttgagcatgttgcccaatttctatgagcctcagttttcttatctaaaaaatgaaactaataatgcctactccaaagaattgttggatggattaagtaagacaacctaatataattgctgtgcatagtaaatcctaactgttggttgccttatacctctaatcttctttctgctctcttttctttagaagatggcatgcatcttttccaaggtaaattttacctctgttgaactctatcccatactctcttgccatggccctaattttgttcagttacttaactccttcccttccctctgacatccttggtcccttcctctccacagttttattctctcgtcttacacaaacatgcccaagtctcatcatccataaaaacctctgtcattaccacttcagctctcttcttctgttcatacccagctgtgttgggtgccatgaatcattcactactattatttttaaaattcctatcactacttctcccctctgcaatctggcttctcctgccaacatgctactgaaattaatgaattaattcatcaaatatttacaagcatttatcatgatactatgt

SEQ ID NO: 57 - DNA, 800 bp, synthetic construct
  source 1..800: mol_type = other DNA
cacaggcactgtgctcagtaactcaagtcattataatactgtgtgcttgacaatatgttgtacttactggccaaaaatcaccaatgtctaattgtcaagctaaatgtcttctttcaatttcaatctatctgacatgctggaaacttccctttccaaggcttctttgctatcgctcccttggctcttctgttcctttgatcttgctgtatttactttgtagtatcctcctaattatgggggtgattccctaggcatccatcttcagctttcttctctctcactcttttaactttactctgtgaatttcctgtaattatacactacatttggtgtgtgtgtgtgtgtgtctgtgtgcacgtgcatttttctaggaaggagttcatagcttttatcagctctgcaaggaggtctatgaccctcccaataacaaccctgaccagttccacctcacgtgtagacacattcttccactgtgatgacttccattatcctccacatgctgctgattcctggatctccaaccctaaacctctttcccatgctccagcgaccatcttcccaactctgcctaggtccctcctggagggtccttctagaataatagttctcatcaggcaggtggcagaacctctgtctcccactccttgcatgcccctctgtgaggcttgggtgggttgggggtgatgggaggggtgcgtgtataatcagagtccctggggtgcagaagctctgttctgaccttgcctcttgctatccatggaattactgctctatcctggccattttcccattctctttct

SEQ ID NO: 58 - DNA, 800 bp, synthetic construct
  source 1..800: mol_type = other DNA
gaagccgggtgtggtggtgcatgcctgtggtcccagccacttgggaaactgaggtgggaggattgctcaagcccaggaggtcaaggctgctgtgagctatgattgtgacactgcactccagcctgggcaacaaagggagaccccttaactaactaaatgaatgaatgaatgaatgaatgaaggcaacctgagctgcattccttggcctgccaacctgcccagccccatccctcagccctccctgagtctgagggccctgcaggtcccacacagggccaggctccatcttgtttctgcaaatttgcaccttccgttccattccctgccacactgctcacgggtcaccatggatgctggataactgccaccgtctccttggagaagccctccgtcatctcagcccctgatgtcaggtattcaacctgcaaatcctccctgtaggtgctgggctctccagagccctgggctgtgtgcaggggactcaggggagaacgggccccacccagctccttcctcacagagctttcagaggctgggggcctccctgatcccctcccagatcccaaggcccctgctgcccccaccctgcagactgggactccgtgaggctgggctctgacttggatattgtggttccagcacacagcaggcaccgtggctgtagtaggcgtgcatgggaagtcaggaggagcagacctgtcatctcccctgctgaggacacagcccggtcagcgtgtcttggcggcctggggccagtgactgagcctcttggcccagttccttgcccaccttgaagatct

SEQ ID NO: 59 - DNA, 800 bp, synthetic construct
  source 1..800: mol_type = other DNA
gtagggagggataaagagatcaggtccaaggccaggtgcggtggttcacgcctgtaatcccagcactttgggaggccgagccaggcagatcgcgaggtcaggagttcgagacgagcctggccatcatggtgaaaccccgtctctactaaaaatacaaaaattggccaggcatggtggcaggcgcctgtaatcccagctgaggcaggagaatcgcttgaacccgggaggtggaggttgcagtgagccgagagcgagccactgcactcctgcctgggtaacaaagcaagactccatctcaaaaaaaaaaaaaaaaaaaaaaatcaagtctggcgcagccccaggaagcaatcttgggctgggcacaccgcatttctgtgacatgaaggcagcgacacctcgtcgctgtcacagggttgctggggttgggggctagaggagaggaggagcctctgttgagggctccaagaagggacagagaggtggtgctcatgggtcctggagacaccttttgggtggttcgtgccctccatcctgggccactttggggaggtgaaggagggaagcattaagggacaagaccccccgttcccaatttctctccggagccagggtttctctgattcgaagaaacaggtgtcacaacccaggaagtccactgatggcatctgccctggggcatcagcatttagggctgatcactgaggtctgcacctcccaaggctgctgtgcccattcctgggcgccccaaaggggaagaaaaactcctgaatgtgcaccgggacaggacccatcccatgcgg

SEQ ID NO: 60 - DNA, 800 bp, synthetic construct
  source 1..800: mol_type = other DNA
agaaaattgccacctattgtattcaccatatgccaggcattgggctgggcacttgagtttatttttctaattcaagaacaaggccaggcacggtggctcatgcctgtaatcccagcactttgggaggctaaggcgggcagatcacaaggtcaggagtttgagaccagcctggccaacatggagaaatcccatctctactaaaaatacaaaaattagctgggtttcgtggtgtgcacctgtagtcccagctactcagggggctgaggcaggagaatctcttgaacccaggaggcggaggttgcagtgagccgagatcgcgccactgcactccagcctgagtgacagagtgagactccgtctaaaaaaaaagaacaaaagccgggcatggtggctcaagcctgtaatcccagcactttgggaggccaaggtgggcagatcatgaggtcagtagttccagaccagcctgaccaacatggtgaaaacctgtttctactaaaaatacaaaaattagctgggcgtggtggcaggcacttgcaatcccagctactcaggaggctgcagcaggagaatcacttgaacccgggaggcagaggttttagtgagctgagatcccgccactgcactccaacctgggcaacagagcaagactctgtctcaaaaaaaaaaaaaaagaaaaaagaaaaaaagaaaagaaaaaagaactaaaagagagtggagcacatttggcatgtgactatagtcccagctacttggtaggctgaggtgagaggatcacttgagccctggagtttgagcccagc

SEQ ID NO: 61 - DNA, 800 bp, synthetic construct
  source 1..800: mol_type = other DNA
ctaggcagtgctgcaagaaccagtctctaaacaaacaaagaaaaaaaaacaggaaaaaaaaagagttatcctagcctagagaaatgttaacagtctatccccttaagatcataaaagtgaaataactggacagagtactgaaaaaagaagaaatctgttgaacaagtctaagtgatttgtagagtctctccaaaggacagtaaagatttcagaaccatggggaggaggctttcttgcagggtctgatgtatacctctagttctatttttagatatattccattgtgctctcttcatttgctcaccattagcatgggaattattccccttgttttatttatatttgtagtaacaagtctagcacagtaactggcatagaagtacttaataaatgttacaatgtgttgaagacttttgagatgtagaattaaattatttgaactacgtttcaggaagttgactctggaagctgtgtgatgaatgaatttagtgactaggagattaggcaaagagagcaattgggaggccacggcaatgatccagacaagagaagtctcagaactgtaatatgacattagcagcagggtagaaagaaggaaatacaaggtatatttcttcaggctttaggattaattggatttaggatgaaaggaaagagtaaattaaatccagtttcttggtgacatgagtgatgtcattaacctgtgacataggaaacactggaagaaaagcaggtttgaggtttaaaataatgttaaacaggccgggagcggtggctcacgcctgtaatcccaacatttt

(19) United States
(12) Patent Application Publication
(10) Pub. No.: US 2026/0209699 A1
(43) Pub. Date: Jul. 23, 2026

(54) ENGINEERED CELLS FOR THE PRODUCTION OF CD4+ T CELLS AND CD4+ TREGS

(71) Applicant: BlueRock Therapeutics LP, Cambridge, MA (US)

(72) Inventors: Mathias Pawlak, Boston, MA (US); Giulia Notarangelo, Boston, MA (US); Chad Dufaud, Cambridge, MA (US)

(21) Appl. No.: 19/138,465

(22) PCT Filed: Dec. 29, 2023
(86) PCT No.: PCT/US2023/086555

§ 371 Date: Jun. 12, 2025

Related U.S. Application Data
(60) Provisional application No. 63/477,910, filed on Dec. 30, 2022.
(60) Provisional application No. 63/477,914, filed on Dec. 30, 2022.

(51) Int. Cl. C12N 5/0783; A61K 35/17; C07K 14/47; C07K 14/705

(52) U.S. Cl. CPC C12N 5/0636; A61K 35/17; C07K 14/4705; C07K 14/705; C07K 14/7051; C07K 14/70525; C07K 14/70532; C07K 14/70539; C12N 5/0637; C12N 2506/45; C12N 2510/00

(57) ABSTRACT

This disclosure relates to engineered cells, artificial antigen presenting substrates (aAPSs), compositions and methods useful for producing CD4+ T cells and CD4+ Tregs from stem and/or progenitor cells. In particular, the disclosure relates to engineered cells, aAPSs, compositions and methods thereof, that can be used to facilitate CD4+ T cell formation. This disclosure also provides for the use of cells for treatment of patients with disease or in need of immune suppression.

### CROSS-REFERENCE TO RELATED APPLICATIONS

This International Patent Application claims the benefit of U.S. Provisional Patent Application No. 63/477,910, filed Dec. 30, 2022, and U.S. Provisional Patent Application No. 63/477,914, filed Dec. 30, 2022, both of which are hereby incorporated by reference in their entireties.

### BACKGROUND

Millions of people worldwide suffer from autoimmune diseases (e.g., type-1 diabetes). Autoimmune diseases arise when the body's natural immune system fails to differentiate between a subject's own healthy cells and foreign or unhealthy cells. This dysregulation of the immune system can lead to life-threatening complications or even death.

In recent years, there has been an increase in attention on the use of Regulatory T cells (Tregs), a specialized subpopulation of CD4+ T cells that act to suppress immune responses, to treat autoimmune disease. One approach is Treg cell transfer, which involves delivering an activated and expanded population of a subject's own Tregs to establish immune homeostasis and self-tolerance. Treg cell transfer therapies have been tested in subjects with type I diabetes with some success, and several clinical trials are ongoing in which Tregs are adoptively transferred to patients with autoimmune disease or recipients of organ transplants with promising results.

Despite tremendous potential for treating autoimmune disease, patients with GvHD (graft versus host disease), or recipients of organ transplants to prevent rejection, reliable sources of Tregs are lacking. Unfortunately, sources of Tregs for cell transfer are generally limited small quantities of cells isolated from blood (e.g., whole blood or apheresis products) and tissue (e.g., thymus), Isolation of cells from blood and tissue is invasive and time-consuming and can yield only small numbers of Tregs, and the expansion of these Tregs ex vivo to obtain the cell numbers needed for a therapeutic benefit is technically challenging. Moreover, Tregs obtained from autogenic sources are often polyclonal in nature and thus can introduce variability in their immunosuppressive response, which can have undesired effects. Thus, methods for producing Tregs in vitro could be advantageous. However, due to the spatial-temporal complexity of Treg development, as well as their phenotypic plasticity, systems for efficient generation of Tregs in vitro have thus far eluded investigators, thus hampering the therapeutical potential of Treg cell therapies.

### SUMMARY

This disclosure provides engineered cells, compositions and methods for generating CD4+ T cells and CD4+ T cell subtypes (e.g., Tregs) from stem and progenitor cells in vitro. In particular, this disclosure provides engineered cells that are useful for producing CD4+ T cells from stem or progenitor cells, as well as compositions and methods that modulate specific interactions between antigen presenting molecules and antigen recognition receptors to enhance formation of CD4+ T cells and Tregs for therapeutic use.

Aspects of this disclosure can leverage attributes of stem and progenitor cells, e.g., the ability to undergo many rounds of cell division, to rapidly and efficiently produce CD4+ T cells. The stem or progenitor cells engineered according to aspects of this disclosure carry transgenes that, when activated, facilitate the formation of CD4+ T cells and CD4+ Tregs. The stem or progenitor cells can also be endowed with antigen-specific moieties, e.g., engineered T cell receptors (TCRs) or chimeric antigen receptors (CARs), which can allow for enhanced immunomodulatory responses at the site of autoimmune activity or even organ transplant.

Other aspects of this disclosure leverage the use of artificial antigen presenting substrates (aAPSs), which can be in the form of artificial antigen presenting cells (aAPCs), to regulate interactions (e.g., duration and strength) between antigen presenting molecules, such as HLA class II molecules, and antigen recognition receptors, such as T cell receptors (TCRs) of emerging progenitor T cells (e.g., DN3-4 or DP cells), to give rise to CD4+ T cells and Tregs in vitro. As disclosed herein, the duration and strength of interactions can be tightly controlled through co-culture or removal of the aAPSs while signal strength can also be controlled through the ratio of progenitor T cells to aAPSs or by the extent of surface expression of the antigen presenting molecules, the nature of the antigen presented and/or antigen recognition receptors. Furthermore, as described herein, the CD4+ T cells generated according to various aspects of this disclosure can be further processed to give rise to various T cell subtypes (e.g., Tregs), which can be used to regulate immune responses and treat disease.

In one aspect, provided herein is an engineered progenitor T cell comprising a heterologous nucleic acid that, when activated, results in the expression of a lineage commitment factor at a level that is higher than that of a comparable wild-type cell to thereby facilitate differentiation towards a CD4+ T cell.

In some embodiments, the engineered progenitor T cell comprises a CD4+CD8+ double positive (DP) phenotype. In other embodiments, the engineered progenitor T cell comprises a CD4−CD8− double negative (DN) phenotype.

In some embodiments, the heterologous nucleic acid is expressed by the engineered progenitor T cell. In some embodiments, the heterologous nucleic acid encodes the lineage commitment factor. In some embodiments, the lineage commitment factor comprises at least one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK. In some embodiments, wherein the lineage commitment factor comprises ThPOK.

In some embodiments, the heterologous nucleic acid is integrated in the genome of the engineered progenitor T cell in a sustained transcriptionally active payload region (STAPLR) or a sustained transgene expression locus (STEL). In some embodiments, the heterologous nucleic acid is integrated into a STAPLR. In some embodiments, the STAPLR is selected from the group consisting of: the intergenic region between the RPL34 gene and the OSTC gene; the intergenic region between the ACTB gene and the FSCN1 gene; the intergenic region between the AKIRIN1 gene and the NDUFS5 gene; the intergenic region between the PRDX1 gene and the AKR1A1 gene; the intergenic region between the PTGES3 gene and the NACA gene; the intergenic region between the MLF2 gene and the PTMS gene; the intergenic region between the RAB13 gene and the RPS27 gene; the intergenic region between the JTB gene and the RAB13 gene; the intergenic region between the AKR1A1 gene and the NASP gene; the intergenic region between the NDUFS5 gene and the MACF1 gene; the intergenic region between the SRSF9 gene and the DYNLL1 gene; the intergenic region between the MYL6B gene and the MYL6 gene; the intergenic region between the GPX1 gene and the RHOA gene; the intergenic region between the HNRNPA2B1 gene and the CBX3 gene; the intergenic region between the ROMO gene and the RBM39 gene; and the intergenic region between the PA2G4 gene and the RPL41 gene. In some embodiments, the STAPLR is the intergenic region between the PRDX1 gene and the AKR1A1 gene. In some embodiments, the heterologous nucleic acid is integrated at a location that is at least 100-5000 base pairs away from the nearest gene.

In some embodiments, the heterologous nucleic acid is integrated into the genome of the engineered progenitor T cell at or near a gene that is specifically expressed in a T cell. In some embodiments, the heterologous nucleic acid is integrated in the genome of the engineered progenitor T cell at or near a TRAC locus.

In some embodiments, the heterologous nucleic acid further comprises an inducible promoter upstream of the lineage commitment factor. In some embodiments, the heterologous nucleic acid, when activated, results in at least a 2-fold increase in expression of the lineage commitment factor as compared to a comparable wild-type cell under similar conditions and thereby steers differentiation of the engineered progenitor T cell into the CD4+ T cell.

In some embodiments, the engineered progenitor T cell further comprises a heterologous nucleic acid sequence encoding a second and optionally a third lineage commitment factor. In some embodiments, the second lineage commitment factor and optionally the third lineage commitment factor, when activated, promotes differentiation towards a CD4+ T cell or a CD4+ T cell subtype. In some embodiments, the T cell subtype comprises a Treg cell.

In some embodiments, the engineered progenitor T cell is derived from an induced pluripotent stem cell. In some embodiments, the induced pluripotent stem cell is derived from a T cell comprising an autoantigen specific TCR.

In some embodiments, the engineered progenitor T cell is genetically modified to integrate an exogenous nucleic acid encoding a T cell receptor (TCR) or a chimeric antigen receptor (CAR). In some embodiments, the exogenous nucleic acid encodes the TCR and the TCR is specific to an autoantigen. In some embodiments, the exogenous nucleic acid is integrated within a STEL. In some embodiments, the STEL comprises a housekeeping gene.

In some embodiments, the housekeeping gene comprises GAPDH. In some embodiments, the exogenous nucleic acid is integrated into a TCR alpha constant (TRAC) locus.

In another aspect, provided herein is an engineered cell comprising a heterologous nucleic acid comprising a coding sequence for a lineage commitment factor that promotes differentiation of the engineered cell towards a CD4+ T cell, wherein the heterologous nucleic acid is integrated within the genome of the engineered cell in a sustained transcriptionally active payload region (STAPLR) or a sustained transgene expression locus (STEL).

In some embodiments, the heterologous nucleic acid is integrated at a location that is at least 100-5000 base pairs away from the nearest gene, and wherein the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

In some embodiments, the engineered cell is further modified to reduce the expression of a competing lineage commitment factor, and wherein the competing lineage commitment factor optionally comprises Runx3.

In yet another aspect, provided herein is an engineered cell comprising a heterologous nucleic acid that, when activated, results in at least a 2-fold increase in expression of ThPOK as compared to a comparable wild-type cell under similar conditions, thereby promoting differentiation of the engineered cell towards a CD4+ T cell.

In some embodiments, the heterologous nucleic acid comprises a coding sequence for ThPOK. In some embodiments, the heterologous nucleic acid further comprises an inducible promoter upstream of the coding sequence.

In some embodiments, the engineered cell further comprises at least one heterologous coding sequence for a lineage commitment factor that, when activated, promotes differentiation of the CD4+ T cell subtype. In some embodiments, the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, or c-Myb.

In some embodiments, the engineered cell comprises one of an embryonic stem cell, an induced pluripotent stem cell, a hematopoietic stem cell, a lymphoid progenitor cell, or a progenitor T cell. In some embodiments, the engineered cell is a stem cell that is engineered to express a TCR that recognizes an autoantigen upon differentiation into a T cell.

In yet another aspect, provided herein is an artificial antigen presenting substrate (aAPS) comprising an immunomodulatory polypeptide.

In some embodiments, the immunomodulatory polypeptide comprises an antigen presenting molecule. In some embodiments, the antigen presenting molecule is linked to an antigen by a peptide. In some embodiments, the antigen presenting molecule comprises a major histocompatibility complex (MHC) molecule. In some embodiments, the MHC molecule comprises an MHC-class II molecule. In some embodiments, the MHC-class II molecule comprises at least one of an HLA-DP molecule, an HLA-DM molecule, an HLA-DO molecule, an HLA-DQ molecule, or an HLA-DR molecule.

In some embodiments, the aAPS comprises an artificial antigen presenting cell (aAPC), a bead, a particle, or a nanoparticle. In some embodiments, the aAPS comprises the aAPC. In some embodiments, the aAPC comprises a cell that is immobilized to a solid surface.

In some embodiments, the aAPS further comprises at least one co-stimulatory molecule or a Notch ligand. In some embodiments, the aAPS comprises the at least one co-stimulatory molecule, and wherein the at least one co-stimulatory molecule comprises at least one of CD40, CD80, CD86, ICOS-L, CD58, or ICAM1. In some embodiments, the co-stimulatory molecule comprises CD80 or ICAM1. In some embodiments, the aAPS comprises the Notch ligand, and wherein the Notch ligand comprises DLL4.

In some embodiments, the aAPS is engineered to secrete one or more cytokines or growth factors. In some embodiments, the one or more cytokines or growth factors are selected to influence cell fate of a target cell into a CD4+ T cell or regulatory T cell. In some embodiments, the one or more cytokines or growth factors comprises TGF-b.

In yet another aspect, provided herein is a composition comprising: a stem cell, any of the engineered progenitor T cells provided herein, or any of the engineered cells provided herein, and any of the aAPSs provided herein.

In yet another aspect, provided herein is a composition comprising any of the aAPSs provided herein, and cell culture media. In some embodiments, the composition further comprises a stem or progenitor T cell that expresses, or is capable of expressing upon differentiation into a T cell, an antigen recognition receptor. In some embodiments, the antigen recognition receptor comprises a TCR. In some embodiments, the antigen of the aAPS is bound by the TCR of the stem or progenitor T cell.

In yet another aspect, provided herein is a method of producing a CD4+ T cell, the method comprising: combining a stem or progenitor T cell that expresses an antigen recognition receptor, or is capable of expressing said antigen recognition receptor upon differentiation into a T cell, with an aAPSs provided herein; differentiating the stem or progenitor T cell towards a CD4+ T cell; and contacting the antigen presenting molecule of the aAPS with the antigen recognition receptor of the T cell thereby promoting differentiation of the stem or progenitor cell towards a CD4+ T cell.

In some embodiments, the combining comprises: adhering the aAPS to a surface of a tissue culture dish; and adding the stem or progenitor T cell to the tissue culture dish comprising the aAPS.

In yet another aspect provided herein is a method of making a CD4+ T cell, the method comprising: activating a heterologous nucleic acid comprising a coding sequence for a lineage commitment factor within an engineered CD4−CD8− double negative (DN) cell or an engineered CD4+CD8+ double positive (DP) cell, wherein the activating results in an increased expression of said lineage commitment factor within the engineered DN cell or the engineered DP cell as compared to a comparable wild-type cell under similar conditions and thereby promotes the differentiation of the engineered DN cell or the engineered DP cell towards a CD4+ T cell.

In some embodiments, the activating occurs in the engineered DN cell. In some embodiments, the activating occurs in the engineered DP cell.

In some embodiments, the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK. In some embodiments, the lineage commitment factor comprises ThPOK.

In some embodiments, the heterologous nucleic acid comprises an inducible promoter upstream of the lineage commitment factor.

In some embodiments, the activating comprises activating the inducible promoter upstream of the coding sequence. In some embodiments, the activating comprises contacting the DN cell with doxycycline or a derivative thereof. In some embodiments, the activating comprises contacting the DP cell with doxycycline or a derivative thereof.

In some embodiments, the DN or DP cell is derived from an induced pluripotent stem cell.

In some embodiments, the method further comprises overexpressing the lineage commitment factor at least 2-fold greater within the engineered DN or DP cell as compared to the comparable wild-type cell under similar conditions.

In some embodiments, the DN cell comprises a DN3 or DN4 cell.

In yet another aspect, provided herein is a method of obtaining a population of cells enriched for CD4+ T cells, the method comprising: obtaining a CD4−CD8− (DN) cells or CD4+CD8+(DP) cells; and overexpressing at least one exogenous lineage commitment factor within the DN cells or DP cells as compared to a comparable wild-type cell under similar conditions to thereby produce a population of cells enriched for CD4+ T cells.

In some embodiments, the method is performed with DN cells. In some embodiments, the method is performed with DP cells.

In some embodiments, the method further comprises contacting the DN cells or DP cells with a first artificial antigen presenting substrate (aAPS).

In some embodiments, the method further comprises contacting the DN cells or the DP cells with a second aAPS, wherein the second aAPS is different than the first aAPS.

In yet another aspect, provided herein is a method of treating a subject in need of immunosuppression, comprising administering to the subject a cell derived from any of the engineered progenitor T cells provided herein or any of the engineered cells provided herein. In some embodiments, the cell derived from the engineered cell is a Treg.

In yet another aspect, provided herein is a method of treating a subject diagnosed with a cancer or a subject in need of immunosuppression, comprising administering to the subject a cell derived from the stem or progenitor T cell contained in any of the compositions provided herein. In some embodiments, the cell derived from the stem or progenitor T cell is a Treg.

In yet another aspect, provided herein is a method of treating a subject diagnosed with a cancer or a subject in need of immunosuppression, comprising administering to the subject a CD4+ T cell or a population of cells enriched for CD4+ T cells obtained from any of the methods provided herein.

In some embodiments of any of the methods of treating a subject diagnosed with a cancer or a subject in need of immunosuppression provided herein, the subject has an autoimmune disease or is a recipient of a transplant.

In yet another aspect, provided herein is the use of any of the engineered progenitor T cells provided herein, or any of the engineered cells provided herein, in the manufacture of a medicament in treating a cancer or a subject in need of immunosuppression.

In yet another aspect, provided herein is the use of the stem or progenitor T cell contained in any of the compositions provided herein, in the manufacture of a medicament in treating a cancer or a subject in need of immunosuppression.

In yet another aspect, provided herein is the use of a CD4+ T cell or a population of cells enriched for CD4+ T cells obtained from any of the methods provided herein, in the manufacture of a medicament in treating a cancer or a subject in need of immunosuppression.

### BRIEF DESCRIPTION OF DRAWINGS

FIG. 1 illustrates an exemplary cell lineage pathway for generating CD4+ T cells from stem or progenitor T cells according to aspects of this disclosure. Shown is an exemplary pathway from a stem cell that harbors a nucleic acid encoding a lineage commitment factor to a CD4+ T cell.

FIG. 2 shows a progenitor T cell in combination with an artificial antigen presenting substrate (aAPS), which is in the form of an artificial antigen presenting cell (aAPC). In particular, shown is a progenitor T cell that has been modified to express a lineage commitment factor in combination with an aAPC presenting a cognate antigen to the antigen recognition receptor of the progenitor T cell.

FIG. 3 illustrates an exemplary cell culture procedure for generating CD34+ cells from stem cells.

FIG. 4 illustrates an exemplary cell culture procedure for generating CD4+ T cells from CD34+ cells.

FIG. 5 is a diagram illustrating an inducible expression system. As illustrated, a first component of the expression system is integrated into a STEL locus, e.g., GAPDH. A second component of the expression system is integrated into a STAPLR locus.

FIGS. 6A and 6B show exemplary experimental results confirming inducible ThPOK expression and reporter protein GFP expression from a STAPLR locus in ThPOK-edited iPSCs. In particular, FIG. 6A shows flow cytometry plots of cells expressing GFP reporter protein integrated into the STAPLR locus of iPSCs untreated (left panel) and treated (right panel) with doxycycline (dox). GFP expression is shown along X-axis. TRA-I-60 expression is shown on Y-axis. FIG. 6B shows histograms of control (left panel) and ThPOK-edited iPSCs (right panel) analyzed by flow cytometry for ThPOK protein expression. ThPOK expression is indicated by X-axis. Event count (normalized to mode) is indicated on Y-axis.

FIG. 7 shows exemplary cytogenetic results of ThPOK-edited iPSCs. The data show that ThPOK-edited cells exhibit a normal karyotype.

FIG. 8 shows exemplary flow cytometry results confirming that ThPOK-edited iPSCs maintain expression of pluripotency markers. In particular, FIG. 8 shows six FACS plots of ThPOK-edited iPSCs stained for SSEA3, SOX2, NANOG, OCT4, TRA-1-60, or SSEA4 as indicated.

FIGS. 9A and 9B are exemplary flow cytometry results showing GFP (FIG. 9A) and ThPOK expression (FIG. 9B) of unedited and three ThPOK-edited cell lines. FIG. 9A shows GFP expression of different cell lines following 0, 1, 2, 3, or 4 days (in order) of dox treatment. Each bar represents GFP geometric mean fluorescent expression (MFI), as measured by flow cytometry, following a different time of treatment with dox. The results show GFP is rapidly upregulated in edited cell populations upon addition of dox as soon as 1 day following treatment. The different cell lines of FIG. 9A were stained with an antibody against ThPOK. FIG. 9B shows that ThPOK expression is induced alongside GFP expression, i.e., ThPOK expression is rapidly upregulated upon addition of dox for 1 day.

FIGS. 10A-10D are exemplary flow cytometry results of the cell populations described above (i.e., unedited and ThPOK-edited cells treated with dox for 0, 1, 2, 3, or 4 days) demonstrating induction of ThPOK in iPSCs results in a loss of certain pluripotency makers, e.g., TRA-1-60 and Oct3/4. FIG. 10A shows flow cytometry results after staining cells with an antibody against TRA-1-60. These results show that after two days of dox treatment (days 3 and 4), the frequency of cells that are positive for pluripotency marker TRA-1-60 is reduced. FIG. 10B SSEA-4 expression on the cell populations after staining the cells with an antibody against SSEA-4. These results show that the frequency of cells positive for SSEA-4 remains constant. FIG. 10C shows Sox2 expression on the cell populations described above after staining the cells with antibodies against Sox2. These results show that the frequency of cells positive for Sox2 remains constant. FIG. 10D shows Oct3/4 expression on the cell populations described above after staining the cells with antibodies against Oct3/4. These results show that the frequency of cells positive for Oct3/4 is rapidly reduced upon treatment with dox. Taken together, these results show induction of ThPOK by the addition of dox results in a loss of pluripotency markers TRA-1-60 and Oct3/4, demonstrating that the inducible ThPOK is functional and when expressed in iPSCs causes changes in cell type.

FIG. 11 shows exemplary images of unedited iPSCs during dox treatment. In particular, shown are panels of microscopic images of iPSCs treated with dox for 0, 1, 2, 3, or 4 days, as indicated.

FIG. 12 shows exemplary images of ThPOK-edited iPSCs during dox treatment. In particular, shown are panels of microscopic images of ThPOK-edited iPSCs treated with dox for 0, 1, 2, 3, or 4 days, as indicated.

FIG. 13 shows exemplary flow cytometry results of iPSCs (unedited) following CD34+ differentiation.

FIG. 14 shows exemplary flow cytometry results demonstrating that premature induction of ThPOK (e.g., before day 14 of T cell differentiation) can result in reduced expression of CD7. In particular, FIG. 14 shows flow cytometry profiles of ThPOK-edited cells in which ThPOK had been induced during T cell differentiation during the time window indicated, e.g., between days 2-21; between days 7-14; between days 14-21, of T cell differentiation.

FIG. 15 shows exemplary flow cytometry results demonstrating premature induction of ThPOK (e.g., before day 14) results in reduced Notch receptor 1 (Notch1) expression. Notch signaling is required for T cell development. The data show that in samples of cell cultures not treated with dox, populations of CD7+ cells are positive for Notch1 expression; however, in samples from cultures treated with dox (e.g., during days 2-21; during days 7-14; or during days 14-21) CD7+ cells are negative for Notch1 expression.

FIG. 16 illustrates a construct that was used to integrate a TCR into an iPSC at a STEL locus (GAPDH locus). The construct comprises P2A-TCRalpha-T2A-TCRbeta-IRES-mCherry sequence components.

FIG. 17 shows exemplary microscope images of TCR-edited iPSCs. The left panel is a fluorescent image of a colony of GAPDH-TCR engineered iPSCs expressing mCherry, which demonstrates the TCR construct is integrated and expressed in the iPSCs. The right panel is a brightfield image of the cell colony from the right panel.

FIG. 18 shows exemplary cytogenetic results of GAPDH-TCR engineered iPSCs. The data show the GAPDH-TCR engineered iPSCs exhibit a normal karyotype.

FIG. 19 shows histograms from a flow cytometry analysis of GAPDH-TCR engineered iPSCs stained for pluripotency markers. In particular, these results show two GAPDH-TCR engineered lines that were unstained (FMO) or stained (Full stain) for SSEA-3, Oct3/4, and TRA-1-60. These results demonstrate GAPDH-TCR engineered iPSCs were positive for pluripotency markers thus demonstrating the GAPDH-TCR engineered iPSCs maintain pluripotency.

FIG. 20 shows exemplary flow cytometry profiles of different aAPCs expressing DLL4, HLA-DR, CD80, and ICAM1. In particular, shown are flow cytometry profiles of unstained aAPCs (control), aAPCs transduced with DLL4 only (APC1); aAPCs transduced with DLL4 and HLA-DR (APC2); aAPCs transduced with DLL4, HLA-DR, and CD80 (APC3); aAPCs transduced with DLL4, HLA-DR, and CD80, ICAM1 (APC4).

FIGS. 21A and 21B shows an exemplary flow cytometry analysis of CD5+ cells isolated at day 15 of T cell differentiation on an artificial antigen presenting substrate. The data show that, following 15 days of differentiation, 67.9% of CD5+ cells (cell line 1, FIG. 21A) or 56.6% (cell line 2, FIG. 21B) of CD5+ cells are CD4+ and CD8alpha.

FIGS. 22A and 22B shows an exemplary flow cytometry analysis of CD5+ cells isolated at day 20 of T cell differentiation on an artificial antigen presenting substrate. The data show that, following 20 days of differentiation, 94.2% (cell line 1, FIG. 22A) of CD5+ cells or 93.3% (cell line 2, FIG. 22B) of CD5+ cells are CD4+ and CD8alpha-.

FIG. 23 shows exemplary results of the expansion capacity of iPSCs derived from T cells (T-iPSCs) and iPSCs derived from other cell types (non-T-iPSCs).

FIGS. 24A and 24B show exemplary results from flow cytometry analyses for a T cell-derived iPSC clone (Cell Line 1), nucleofected with mRNA encoding ThPOK (ThPOK mRNA), without mRNA (No mRNA), or not nucleofected (No EP). FIG. 24A shows the expression of ThPOK, CD4 and CD8 as measured by flow cytometry, while FIG. 24B shows the level of expression of Runx3 as measured by flow cytometry.

FIGS. 25A and 25B show exemplary results from flow cytometry analyses for a T cell-derived iPSC clone (Cell Line 2), nucleofected with mRNA encoding ThPOK (ThPOK mRNA), without mRNA (No mRNA), or not nucleofected (No EP). FIG. 25A shows the expression of ThPOK, CD4 and CD8 as measured by flow cytometry, while FIG. 25B shows the level of expression of Runx3 as measured by flow cytometry.

### DEFINITIONS

The following definitions supplement those in the art and are directed to the present disclosure only. The following definitions are not to be imputed to any related or unrelated case, e.g., to any commonly owned patent or patent application. Although some methods and materials similar or equivalent to those described herein can be used to practice features of the disclosure, some preferred materials and methods are described herein. Accordingly, the terminology used herein is for the purpose of describing particular embodiments only and is not intended to be limiting.

As used herein, the singular form “a”, “an”, and “the” include plural references unless the context clearly dictates otherwise. For example, the term “a cell” includes a plurality of cells, including mixtures thereof.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “activating”, and its grammatical equivalents, refers to inducing or enhancing an activity of a cell or a component thereof. By way of example, and without limitation, activating refers to an activity that results in an increased expression of a nucleic acid within a target cell. For example, activating an activity of a cell can make use of an inducible gene expression system (e.g., a tetracycline/doxycycline-controlled transcriptional activation system) and can be accomplished by contacting the target cell with a compound such as tetracycline, or a derivative thereof such as doxycycline, to cause the binding of an activator protein, e.g., a transcriptional transactivator (tTA) protein, to a regulatory sequence of the nucleic acid to be expressed by the target cell. The binding of the activator protein to the regulatory sequence, e.g., a promoter, can result in the increased expression of the nucleic acid as compared to a comparable nucleic acid that has not been activated. In some instances, activating refers to an activity that results in an expressed expression of a protein within a cell. For example, activating an activity of a cell can involve incorporating nucleic acid, e.g., RNA, encoding a protein, e.g., ThPOK, into the cell, which can result in the increased expression of the protein.

The term “administering”, and its grammatical equivalents, refers to providing a composition, e.g., a composition of engineered cells, to a subject or a subject in need thereof. By way of example, and without limitation, administering can be carried out by injection, e.g., by intravenous injection, sub-cutaneous injection, intradermal injection, intraperitoneal injection, or intramuscular injection, of an amount (e.g., a therapeutically effective amount) of an agent, molecule, or cells (e.g., engineered cells as described herein).

The term “antigen”, and its grammatical equivalents, refers to a compound, composition, or substance that can be specifically bound by the products of specific humoral or cellular immunity, such as an antibody molecule or T cell receptor (TCR). Antigens can be any type of molecule including, for example, peptides, haptens, simple intermediary metabolites, sugars (e.g., oligosaccharides), lipids, and hormones as well as macromolecules such as complex carbohydrates (e.g., polysaccharides), phospholipids, and proteins. For example, the term antigen can refer to a substance that induces an immune response. Common categories of antigens include, but are not limited to, viral antigens, bacterial antigens, fungal antigens, protozoan and other parasitic antigens, tumor antigens, antigens involved in autoimmune disease, allergy and graft rejection, toxins, and other miscellaneous antigens. The antigen can comprise an autoantigen. For example, the antigen can be of the subject in need of cellular treatment. The antigen can comprise a foreign antigen. The antigen can be a cognate antigen. A “cognate antigen” refers to an antigen that is recognized by a particular antigen recognition receptor (e.g., TCR) of a progenitor T cell of the disclosure. In some instances, recognition of the cognate antigen by the TCR can facilitate development of a progenitor T cell into a CD4+ T cell.

The term “antigen presenting molecule”, and its grammatical equivalents, refers to a molecule or complex of molecules that can present an antigen to a progenitor T cell or an engineered cell (e.g., an engineered stem or progenitor cell comprising a TCR). For example, without limitations, in some embodiments, the antigen presenting molecule is a major histocompatibility complex (MHC) molecule. In some embodiments, the MHC molecule is an MHC class I molecule. In other embodiments, the MHC molecule is an MHC class II molecule. In humans, MHC molecules are referred to as human leukocyte antigen (HLA) molecules. Thus, in some embodiments, the antigen presenting molecule is an HLA molecule, e.g., an HLA class I or an HLA class II molecule. The antigen presenting molecule can be expressed by an artificial antigen presenting substrate. For example, the antigen presenting molecule can be encoded by a nucleic acid that is transduced into an artificial antigen presenting substrate. The nucleic acid encoding the antigen presenting molecule can also encode an antigen. The sequence encoding the antigen can be linked to a sequence encoding the antigen presenting molecule by a linker. The linker can encode, without limitation, between 3 to 15 peptides. The linker can comprise a plurality of glycine or serine residues.

The terms “artificial antigen presenting substrate” or “aAPS”, and its grammatical equivalents, refers to a genetically modified cell, substrate, or particle engineered to deliver one or more stimulatory signals to a target cell. By way of example, and without limitation, the artificial antigen presenting substrate includes an artificial antigen presenting cell (aAPC), a bead, a particle, or a nanoparticle. The aAPS can be engineered to provide one or more stimulatory signals that induce a signal transduction or changes in protein expression within a target cell and thereby influence cell fate of the target cell. For example, the aAPS can be engineered to deliver one or more stimulatory signals that facilitate the differentiation of a target cell (e.g., a stem or progenitor T cell comprising a TCR) into a CD4+ T Cell. The one or more stimulatory signals can include signals that stimulate antigen specific T cells. For example, a first signal can involve a major histocompatibility complex (MHC) molecule, which in humans is also referred to as the human leukocyte antigen (HLA). This molecule can be loaded with a specific antigen or epitope to be presented to antigen-specific T cells. The peptide-loaded MHC can then engage with a cognate T cell receptor (TCR), which can be found on the target cell. In some instances, an APS of the disclosure further includes a co-stimulatory molecule, e.g., protein CD80 or CD86. In some instances, an APS of the disclosure further includes an adhesion molecule, e.g., protein ICAM-1. In some instances, an aAPS can be further engineered to secrete one or more stimulatory cytokines such as IL-2, which enhances T cell stimulation and expansion, or growth factors such as TGF-beta. In some instances, an APS is further engineered to express a human Notch ligand (DLL4) on its surface.

The term “antigen recognition receptor”, and its grammatical equivalents, refers to a structure or complex that is present on a surface of a biological system (e.g., a cell) and can receive and initiate a signaling pathway in the biological system. For example, and without limitation, an antigen recognition receptor can be a T cell receptor (TCR) or a chimeric antigen receptor (CAR).

The term “cell”, and its grammatical equivalents, refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoan cell, a cell from a plant, a fungal cell, an animal cell, a cell from a mammal, and the like. Sometimes a cell is not originating from a natural organism (e.g., a cell can be a synthetically made, sometimes termed an artificial cell). In some embodiments, the cell is an adherent cell, e.g., a cell that can be immobilized on a surface of a culture vessel and/or can grow while adhering to a culture vessel. In other embodiments, the cell can be grown in a suspended state, e.g., in a medium.

The term “CD4+ T cell”, and its grammatical equivalents, refers to a type of lymphocyte that is positive for cell surface receptor/marker CD4 (cluster of differentiation 4) and negative for a functional CD8 receptor/marker. As described herein, CD4+ T cells can be differentiated into regulatory T (Treg) cells and conventional T helper (Th) cells. The CD4+ T cells can play a role in cell-mediated immunity and can be distinguished from other types of lymphocytes, such as B cells, by the presence of a TCR on their surface. The CD4 can be described as a co-receptor of a T cell receptor (TCR) and can assist the TCR in communicating with antigen-presenting cells. In some embodiments, CD4+ T cell is negative for surface receptor/marker CD8 alpha. In some embodiments, the CD4+ T cell is negative for surface receptor/marker CD8 beta. In some embodiments, the CD4+ T cell is negative for CD8 alpha and CD8 beta.

The term “CD4+T-regulatory cell”, and its grammatical equivalents, refers to a specialized subpopulation of T cells that can act to suppress immune response, thereby maintaining homeostasis and immune tolerance. By way of example, and without limitation, CD4+T-regulatory cells (as described as Tregs) are able to inhibit effector T cell proliferation and cytokine production and play a critical role in preventing autoimmunity, resolving and/or restraining autoimmunity. As described herein, the CD4+T-regulatory cell can be substantially similar to the type of CD4+T-regulatory cell that matures in the thymus. In some instances, the CD4+T-regulatory cell is positive for one or more of CD25, CTLA-4, GITR, LAG-3, GARP, LAP, Helios, and FOXP3 markers.

The term “coding sequence”, and its grammatical equivalents, refers to a segment of a nucleic acid that encodes a polypeptide. The segment of nucleic acid can be said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The term “composition”, and its grammatical equivalents, refers to a combination of an agent (e.g., an engineered cell) and a naturally-occurring or non-naturally-occurring carrier. For example, the carrier can be inert or active, such as an adjuvant, diluent, binder, stabilizer, buffer, salt, lipophilic solvent, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Carriers also include cell culture media and cell wash solutions.

The term “co-stimulatory molecule”, and its grammatical equivalents, refers to a molecule that delivers or modulates a signal which immune cells can use to activate an immune response in the presence of an antigen-presenting cell. In some instances, the co-stimulatory molecule is provided on the surface of an aAPS. In some instances, the co-stimulatory molecule is any one or more of CD40, CD80, CD83, CD86, ICOS-L, CD58, and ICAM1 (sometimes referred to as CD54). In some instances, an aAPS comprises CD80 and ICAM1.

The term “differentiation”, and its grammatical equivalents, refers to a process by which a stem cell or progenitor cell alters from one cell type to a more specialized cell type. Each specialized cell type in an organism can express a subset of all the genes that constitute the genome of the cell. Each cell type can be defined by its particular pattern of regulated gene expression. Cell differentiation can thus be described as a transition of a cell from one cell type to another cell type coincident with a switch from one pattern of gene expression to another.

The terms “engineered cell” or “genetically modified cell” and their grammatical equivalents, refers to a cell of human or non-human animal origin that cannot be found in nature. As described herein, an engineered cell refers to a cell that has been genetically modified. By way of example, and without limitation, an engineered cell can be genetically modified by the integration of a heterologous nucleic acid within the genome of the engineered cell, which, when activated, can allow the engineered cell to express a protein and/or a polynucleotide at a higher level than that of a comparable wild-type cell. In some embodiments, the engineered cell is a stem or progenitor cell that has been genetically modified. For example, in some embodiments the engineered cell is an induced pluripotent stem cell. In some embodiments, the genetically modified cells are adherent cells.

The terms “exogenous nucleic acid”, “heterologous nucleic acid”, or “transgene”, and their grammatical equivalents, can be used interchangeably herein and refers to a segment of nucleic acid which has been incorporated into a host genome or is capable of autonomous replication in a host cell. In some instances, the heterologous nucleic acid can harbor one or more coding sequences. Exemplary coding sequences can provide the host cell with a novel phenotype relative to a corresponding wild-type cell. Such heterologous nucleic acids may be, for example, introduced into a host cell by genetic transformation, viral transduction, transfection, or electroporation.

The term “gene”, and its grammatical equivalents, refer to a nucleic acid (e.g., DNA such as genomic DNA and cDNA) and its corresponding nucleotide sequence that can be involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region can contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which can be introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The terms “genetic modification”, or “genetically modified” and their grammatical equivalents, refer to a modification or manipulation of a cell's genetic material. The genetic modification can be accomplished using any number of a set of technologies known in the art that are used to change the genetic makeup of cells, including the transfer of genes within and across species boundaries to produce improved or novel organisms. A cell comprising a genetic modification can have an altered genetic structure caused by either removing or introducing one or more nucleotides. In some embodiments, the genetic modification can refer to an alteration of a nucleic acid within the cell. By “alteration” is meant a change (e.g., an increase or decrease) in the structure, expression levels or activity of a gene or polypeptide as detected by standard art known methods such as those described herein. As used herein, an alteration includes a change in a polynucleotide or polypeptide sequence or a change in expression levels, such as a 10% change, a 25% change, a 40% change, a 50% change, or greater.

The term “genome”, and its grammatical equivalents, refers to a complete set of genes or genetic material present in a cell or organism.

The term “immunosuppression”, and its grammatical equivalents, refers to the partial or complete suppression of an immune response of a subject or subject. In some embodiments, immunosuppression is induced to help the survival of a subject or subject after a transplant operation.

The term “intergenic region”, and its grammatical equivalents, refers to a stretch of nucleotide sequences located between two neighboring genes. When referencing an intergenic region, the entire region does not need to be utilized; nor does the present disclosure always refer to the entire intergenic region when using the term “intergenic region.” For instance, the traditional sense of intergenic region is that there are two genes which are separated by a series of nucleotides; that is, two genes flank the intergenic region. However, as gene databases are being updated, not all adjacent genes are consistently shown to be demarcated by an intergenic region. There are a continuing number of overlapping genes being reported, where an “overlapping gene” is a series of nucleotides encoding one gene partially intertwines, or overlaps on the same or opposing DNA strand, with a series of nucleotides which encode another gene. With this understanding, there are situations where an intergenic region can include a fragment of a particularly described transcript of an overlapping gene in a particular database or with a different cell line. Intergenic regions may contain functionally important elements, such as promoters or enhancers, yet not all intergenic regions comprise such elements. Nearby genes may be thousands of base pairs from the functional area of the intergenic region or can be immediately adjacent to the intergenic region. Additionally, the intergenic region can be of various size. For example, the intergenic region can be at least 100 base pairs in length. In other embodiments, the intergenic region can be at least 150 base pairs in length. In other embodiments, the intergenic region can be at least 200 base pairs in length. In some embodiments, the intergenic region is at least 300 base pairs, at least 400 base pairs, at least 500 base pairs, at least 1000 base pairs, at least 1500 base pairs, at least 2000 base pairs, at least 2500 base pairs, at least 3000 base pairs, at least 3500 base pairs, at least 5000 base pairs, at least 10000 base pairs, at least 15000 base pairs, at least 20000 base pairs, at least 30000, at least 40000, at least 50000, at least 75000, or at least 100000 base pairs in length.

The term “lineage commitment factor”, and its grammatical equivalents, refers to a cellular component (e.g., a protein, a protein fragment, a polypeptide, a polynucleotide, etc.) that can influence the differentiation of a cell. By way of example, and without limitation, a lineage commitment factor can refer to a protein or protein fragment that, when expressed by the cell, promotes the differentiation of the cell (e.g., an iPSC) into a more specialized cell type (e.g., a CD4+ T cell). In some embodiments, the linage commitment factor is a protein, or protein fragment, of FOXP3, Helios, or ThPOK.

The term “MHC-class II molecule”, and its grammatical equivalents, refers to an antigen presenting molecule. In particular, the term MHC-class II molecule can refer to a class of major histocompatibility complex (MHC) molecules normally found on professional antigen-presenting cells, such as dendritic cells, mononuclear phagocytes, some endothelial cells, thymic epithelial cells, and B cells. MHC-class II molecules can be heterodimers and can consist of an α and β chain, both of which can be encoded in the MHC gene locus. In some embodiments, the chains can include sub-designations, e.g., α1, α2, etc., which can refer to separate domains within the HLA gene. Each domain can be encoded by a different exon within a gene, and some genes can further include domains that encode leader sequences, transmembrane sequences, etc. In some embodiments, these molecules can include both extracellular regions as well as a transmembrane sequence and a cytoplasmic tail. In some embodiments, the α1 and β1 regions of the chains come together to make a membrane-distal peptide-binding domain, while the α2 and β2 regions, the remaining extracellular parts of the chains, form a membrane-proximal immunoglobulin-like domain. The antigen binding groove, where the antigen or peptide binds, is made up of two α-helixes walls and β-sheet. In some embodiments, the MHC class II molecules can deliver antigens to a target cell. Loading of an MHC class II molecule with an antigen can occur by phagocytosis; extracellular proteins are endocytosed, digested in lysosomes, and the resulting epitopic peptide fragments are loaded onto MHC class II molecules prior to their migration to the cell surface. In some embodiments, the MHC-class molecule is an HLA-DP molecule, an HLA-DM molecule, an HLA-DO molecule, an HLA-DQ molecule, or an HLA-DR molecule.

The terms “nucleic acid” and “nucleic acid molecule”, and their grammatical equivalents, refers to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g, nucleotides and/or nucleosides). In some embodiments, “nucleic acid” refers to an oligonucleotide chain comprising three or more individual nucleotide residues.

The term “progenitor cell”, and its grammatical equivalents, refer to a descendant of a stem cell that can further differentiate into specialized cell types within a particular cell lineage. For example, a progenitor cell can be a lymphoid progenitor cell, whereby upon further differentiation give rise to a lymphoblast (the precursor to T cells), or a myeloid progenitor cell, whereby upon further differentiation give rise to a myeloblast (e.g., the precursor to granulocytes). A progenitor cell can be a primary lymphoid progenitor cell. Likewise, a “progenitor T cell” refers to a progenitor cell within the T cell lineage, whereby upon further differentiation give rise to specialized T cells (e.g., effector cells, such as cytotoxic T cells, regulatory T (Treg) cells or T helper (Th) cells, or memory T cells). A progenitor T cell can, for example, further differentiate into a CD4+ T cell. A progenitor T cell can comprise a CD4+CD8+ double positive (DP) phenotype. A progenitor T cell can comprise a CD4−CD8− double negative (DN) phenotype.

The term “promoter”, and its grammatical equivalents, refers to a region of a nucleic acid positioned upstream of a gene where relevant proteins (such as RNA polymerase and transcription factors) bind to initiate transcription of the gene. In some embodiments, the promoter can be a tissue-specific promoter. For example, a promoter of a gene that is turned on or off in certain cell or tissue types. For example, the promoter can be a CD4 gene promoter, or a ThPOK gene promoter. In some embodiments, the promoter can be an inducible promoter. An inducible promoter can refer to a regulated promoter which becomes on or active in a cell in response to specific stimuli, e.g., the binding of an activator protein.

The term “sustained transcriptionally active payload region” or “STAPLR” and its grammatical equivalents, refers to an intergenic region in the mammalian genome that allows consistent levels of expression of transgenes integrated therein, including as the cell undergoes changes in its differentiation state. STAPLR expands the repertoire of genomic safe harbors where transgenes can be stably integrated and their expression can be maintained over multiple passages and as the cell changes its phenotype. The term “payload” or “genomic payload” in the context of STAPLR refers to exogenous or heterologous nucleotide sequences introduced to the region. The STAPLR can refer to an intergenic region found between essential genes or genes that are expressed throughout different cell states.

The term “T cell receptor”, or sometimes identified herein as “TCR”, and its grammatical equivalents, refers to a molecule, a protein, or a protein complex found on the surface of cells that can bind with an antigen or a fragment of an antigen such as a peptide bound to a major histocompatibility complex (MHC) molecules. The TCR can be composed of two different protein chains (that is, it is a heterodimer). In some embodiments, the TCR consists of an alpha (α) chain and a beta (β) chain (encoded by TRA and TRB loci, respectively). In other embodiments, the TCR consists of gamma and delta (γ/≢) chains (encoded by TRG and TRD, respectively). When the TCR engages with antigenic peptide and MHC (peptide/MHC), the cell is activated through signal transduction, that is, a series of biochemical events mediated by associated enzymes (e.g. protein kinases), co-receptors, specialized adaptor molecules, and activated or released transcription factors. Based on the initial receptor triggering mechanism, the TCR belongs to the family of non-catalytic tyrosine-phosphorylated receptors (NTRs).

The term “therapeutically effective amount” as used herein refers to an agent (e.g., an engineered cell) that is sufficient to induce a desired response, which can be used to manage, prevent, or treat a given disease, disorder, or condition as described herein, and/or a symptom related thereto. For example, a therapeutically effective amount when used in reference to cells administered to a subject refers to an amount of cells that, when administered to the subject, causes a detectable level of a T cell response as compared to a T cell response detected in the absence of the administering. The T cell response in a subject can be readily assessed by methods known in the art. As another example, when used in reference to an amount of a compositions, such as a pharmaceutical composition, the term therapeutically effective amount refers to the amount of a pharmaceutical composition, or the number of cells, that when administered to the subject, is sufficient to effect the treatment of the subject. A therapeutically effective amount of an agent of the present disclosure (e.g., an engineered cell) can vary according to factors such as the disease state, age, sex, and weight of the individual, and the ability of the agent to elicit a desired response in the individual. A therapeutically effective amount encompasses an amount in which any toxic or detrimental effects of the agent are outweighed by the therapeutically beneficial effects. A therapeutically effective amount also encompasses an amount of an engineered cell or other agent (e.g., drug) effective to treat a disease, disorder, or condition described herein, in a subject or mammal.

The terms “treating”, and “treatment”, refer to alleviation or elimination of one or more symptoms of the treated condition, prevention of the occurrence or reoccurrence of the symptoms, reversal or remediation of tissue damage, and/or slowing of disease progression.

As used herein, a statement that a cell or population of cells is “positive” for a particular marker refers to the detectable presence on or in the cell of a particular marker, for example, a surface marker or an intracellular marker, such as transcription factors. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker for example, a surface marker or an intracellular marker, such as transcription factors. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, or at a level substantially similar as compared to that for a cell known to be negative for the marker.

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)); Barbare Detrick, Robert Hamilton, John L Schmitz, Manual of Molecular and Clinical Lab Immunology (2016).

### DETAILED DESCRIPTION

The present disclosure provides methods and compositions useful for the efficient generation of CD4+ T cells, and derivatives thereof, from stem and/or progenitor cells including induced pluripotent stem cells. Thus, as those skilled in the art will readily appreciate, this disclosure addresses a long-standing problem in the T cell therapy space, as current state-of-the-art technologies do not allow for the efficient derivation of CD4+ T cells from stem cells. Current T cell therapies are generally restricted to the isolation and expansion of T cells from autologous or haplotype-matched allogeneic sources, which is expensive, time-consuming, and generally limited to the formation of polyclonal T cells, which not only lack the efficacy of antigen-specific T cells but also may have unknown or undesired effects.

Furthermore, since CD4+ T cells are cell lineage precursors to various types of therapeutically relevant T cells, including CD4+ cytotoxic T cells, Th1, Th2, Th17, Tr1, Tfh, Tfr, and T regulatory cells (Tregs), the methods and compositions described herein can be used for the generation of a plurality of T cell subtypes for treating diseases such as autoimmunity, cancer or to reinstate immune system homeostasis.

### I. MODULATING TRANSCRIPTION AND EPIGENETIC PROGRAMS TO GENERATE CD4+T CELLS

In some aspects, this disclosure provides for the efficient generation of CD4+ T cells via controlled changes to transcriptional and epigenetic programs that govern cell fate transitions. For example, this disclosure provides compositions and methods useful for the overexpression (i.e., expression of a gene at a higher level than a comparable wild-type cell) of lineage commitment factors involved in the determination of cell fate choice during the CD4+CD8+ double-positive (“DP”) to CD4 single positive (referred to herein as CD4+ T cells) transition. This disclosure also provides compositions and methods useful for the overexpression of lineage commitment factors during stages of T cell development that precede the DP stage, including, for example, DN stage (e.g., DN3 or DN4). This disclosure also provides for the expression of the transcription factors from specific genomic regions that allow for their stable expression during prolonged cell culture, e.g., five or more cell divisions, and cell type changes that occur during T cell differentiation.

A person skilled in the art will readily appreciate that changes in cell type often accompany changes in genome organization that are coincident with changes in gene expression. One insight of this disclosure is the recognition that many gene-editing technologies practiced in the cell therapy space are incapable of generating CD4+ T cells from stem cells because current technologies do not adequately account for the impact of changes in genome organization on transgene expression during T cell development.

Aspects of this disclosure make use of specific genomic loci (i.e., STEL and STAPLR) that were selected after extensive experimentation for their capacity to permit stable transgene expression (or to permit a desired level of expression upon induction) during prolonged cell culture and T cell development. Stable transgene expression is important for ensuring functionality and optimal protein production during the formation of CD4+ T cells. Accordingly, in some aspects, this disclosure provides stem or progenitor cells, including progenitor T cells, with linage commitment factors (e.g., ThPOK), and optionally transcriptional regulator proteins, that are integrated into a STEL or STAPLR of a genome.

In other aspects, this disclosure provides strategies for controlling the temporal expression of lineage commitment factors during CD4+ T cell development. For example, this disclosure provides compositions and methods with stem or progenitor T cells comprising inducible lineage commitment factors. The stem or progenitor T cells can be induced to express lineage commitment factors at specific stages of T cell development. For example, in some embodiments, stem or progenitor T cells can be induced to express a lineage commitment factor (e.g., ThPOK) during the CD4+CD8+ double positive (DP) stage of T cell differentiation using, for example, a dox-inducible system, to facilitate the generation of CD4+ T cells. In other embodiments, stem or progenitor T cells can be induced to express a lineage commitment factor during the CD4−CD8− double negative (DN) stage of T cell differentiation to facilitate the generation of CD4+ T cells. For example, in some embodiments a lineage commitment factor is induced during the DN3 stage of T cell development. In some embodiments, a lineage commitment factor is induced during the DN4 stage of T cell development. Accordingly, in some embodiments, a CD4+ T cell can be generated by expressing a lineage commitment factor in a progenitor T cell, such as, for example, a DP or a DN cell.

As described herein, the CD4+ T cells can be further differentiated into various T cell subtypes, such as T regulatory cells, which express FoxP3, and Tr cells, which produce IL-10 without expression of FoxP3. These cells can have potential applications in autoimmunity and immune system homeostasis. In addition, pro-inflammatory iPSC-derived CD4+ T cells can be generated according to aspects of this disclosure, which can be useful in areas such as cancer (e.g., Th1, Th2 and cytotoxic CD4+ T cells).

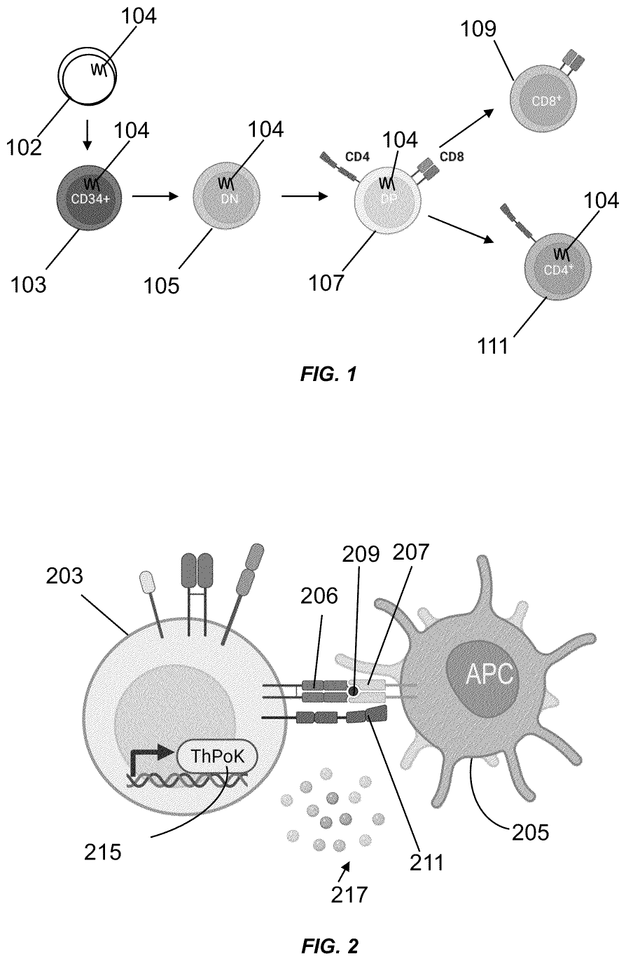

FIG. 1 illustrates an exemplary cell lineage pathway for generating CD4+ T cells from stem or progenitor T cells according to aspects of this disclosure.

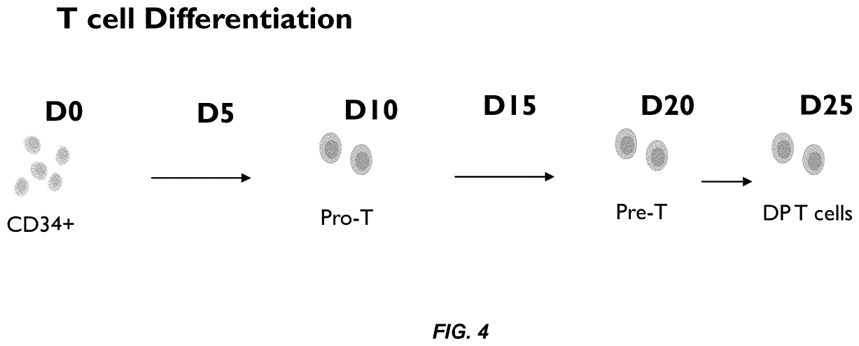

In particular, FIG. 1 shows a CD34+ stem cell 103 that has a heterologous nucleic acid 104 encoding a cell lineage commitment factor (e.g., ThPOK). The stem cell 103 can be derived from a variety of sources, including a pluripotent stem cell 102, such as, an induced pluripotent stem cell, an embryonic stem cell, or multipotent stem cells, such as, a cord blood stem cell. The stem cell 103 can be induced to differentiate, in vitro, from a cell culture dish into a T cell, for example, by following the cell differentiation programs described in detail below (see FIG. 4). During differentiation the stem cell 103 transitions into a “double negative” or “DN” cell 105, which is named in reference to an absence of expression for both CD4 and CD8 surface markers.

After approximately 20-30 days of cell differentiation, the DN cell 105 transitions into a CD4+CD8+ double positive “DP” cell 107. During the DP stage, the heterologous nucleic acid can be activated to thereby steer differentiation of the DP cell 107 towards a CD4+ cell fate 111 and away from a CD8+ cell fate 109. For example, in some embodiments, expression of the heterologous nucleic 104 is regulated in part by a tet/dox-inducible promoter. Accordingly, activating the heterologous nucleic acid to facilitate the formation of a CD4+ T cell 111 can be achieved by contacting the DP cell 107 with, for example, doxycycline.

Accordingly, in some aspects, this disclosure provides a stem or progenitor T cell, for example, a stem cell-derived CD4+CD8+ double positive cell, the ability to express a lineage commitment factor (e.g., ThPOK) at a level that is higher than normally found in T lymphoid development or in an artificial differentiation system that make T cells.

In additional aspects, this disclosure provides compositions that facilitate formation of CD4+ T cells from stem or progenitor cells harboring an inducible, exogenous lineage commitment factor. In some embodiments, the compositions comprise artificial antigen presenting substrates, e.g., artificial antigen presenting cells. In particular, and as described in further detail below, aspects of this disclosure include the insight that interactions between HLA molecules and antigen recognition receptors (e.g., TCRs) of stem or progenitor T cells can be used to facilitate the differentiation of the stem or progenitor T cell into a CD4+ T cell. Accordingly, in some embodiments, engineered stem or progenitor T cells comprising heterologous lineage commitment factors are cultured in combination with artificial antigen presenting substrates (as described further below) to enhance formation of CD4+ T cells.

FIG. 2 shows a progenitor T cell 203 in combination with an artificial antigen presenting substrate (aAPS), which is in the form of an aAPC 205. In particular, illustrated is a progenitor T cell 203 that has been genetically modified to integrate an inducible lineage commitment factor (e.g., ThPOK) 215 within the cell's genome. The antigen recognition receptor (TCR) 206 of the progenitor T cell 203 associates with an HLA molecule 207 (e.g., an HLA class II molecule, such as, HLA-DR) of the aAPC 205 which presents a cognate antigen 209. In some embodiments, the cognate antigen 209 is linked to the HLA molecule, for example, by a linker, to enhance interaction efficacy. In other embodiments, the aAPC 205 is loaded with the cognate antigen 209 by adding cognate antigens 209 to media prior to combining the progenitor T cells 203 with the aAPC 205. Binding of the TCR of the progenitor T cell to the cognate antigen 209 presented by the HLA molecule (in combination with co-stimulatory molecules (e.g., CD80 or ICAMI) 211 can elicit a signaling cascade that facilitates differentiation of the progenitor T cell into a CD4+ T cell. In some embodiments, the aAPC is modified to express a Notch ligand (e.g., DLL4) and/or secrete one or more cytokines (e.g., IL-2, IL-6) or growth factors (e.g., TGF-β) 217 that facilitate T cell differentiation.

### A. Nucleic Acids Encoding Lineage Commitment Factors

This disclosure provides stem and progenitor cells that are engineered with nucleic acids that encode lineage commitment factors. Advantageously, the commitment factors can encode proteins that promote cellular differentiation of the engineered cells towards CD4+T cells, or derivatives thereof, e.g., Tregs. This disclosure also provides engineered cells that can express one or more commitment factors to thereby promote lineage commitment of stem or progenitor cells into CD4+ T cells or CD4+ T cell derivatives. In some embodiments, the lineage commitment factor comprises a CD4+ T cell lineage commitment factor. In some embodiments, the lineage factor comprises a gene that encodes a protein that is upregulated in a CD4+ T cell or a CD4+ T cell subtype. In some embodiments, the engineered cells comprise one exogenous commitment factor. In some embodiments, the engineered cells comprise two exogenous commitment factors. In some embodiments, the engineered cells comprise more than two commitment factors, e.g., at least 3 commitment factors.

In some embodiments, the engineered cell comprises a heterologous nucleic acid encoding at least one of CD4 (Gene ID: 920), CD25 (Gene ID: 3559), FOXP3 (Gene ID: 50943), CD45RA (Gene ID: 5788), CD62L (Gene ID: 6402), Helios (Gene ID: 22807), GITR (Gene ID: 8784), Ikaros (Gene ID: 10320), CTLA4 (Gene ID: 1493), Gata3 (Gene ID: 2625), Tox (Gene ID: 9760), ETS1 (Gene ID: 2113), TCF7 (Gene ID: 6932), LEF1 (Gene ID: 51176), RORA (Gene ID: 6095), TNFR2 (Gene ID: 7133), Eos (Gene ID: 7908), Irf5 (Gene ID: 3663), SatB1 (Gene ID: 6304), Gatal (Gene ID: 2623), c-Myb (4602), or ThPOK (Gene ID: 51043; sometimes referred to as ZBTB7B). For example, in some embodiments, the heterologous nucleic acid comprises a cDNA sequence of at least one of CD4 (Gene ID: 920), CD25 (Gene ID: 3559), FOXP3 (Gene ID: 50943), CD45RA (Gene ID: 5788), CD62L (Gene ID: 6402), Helios (Gene ID: 22807), GITR (Gene ID: 8784), Ikaros (Gene ID: 10320), CTLA4 (Gene ID: 1493), Gata3 (Gene ID: 2625), Tox (Gene ID: 9760), ETS1 (Gene ID: 2113), TCF7 (Gene ID: 6932), LEF1 (Gene ID: 51176), RORA (Gene ID: 6095), TNFR2 (Gene ID: 7133), Eos (Gene ID: 7908), Irf5 (Gene ID: 3663), SatB1 (Gene ID: 6304), Gatal (Gene ID: 2623), c-Myb (4602), or ThPOK (Gene ID: 51043). cDNA sequences can be found on known genome databases, such as NCBI. In some embodiments, the sequence encoding the commitment factor is codon optimized. Codon-optimization describes gene engineering approaches that use synonymous codon changes to increase protein production.

In some embodiments, the engineered cell comprises one or more heterogenous nucleic acids encoding at least two of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK. In some embodiments, the engineered cell comprises one or more heterogenous nucleic acids encoding at least three of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, and ThPOK. For example, in some embodiments, the heterologous nucleic acid encodes three lineage commitment factors where at least one of the commitment factors are selected from CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, and ThPOK. The lineage commitment factors can be encoded on the same heterologous nucleic acid downstream of an inducible promoter and, in some embodiments, separated by self-cleaving peptide sequences such as picorna-virus derived 2A sequences.

In some embodiments, the commitment factor comprises ThPOK. In some embodiments, the engineered cell comprises a heterologous nucleic acid comprising a sequence that has at least 80 percent, at least 85 percent, at least 90 percent, at least 95 percent, at least 99 percent, or has 100 percent identify to:

AGGGAGGGGAGGGATGGGGGGAAAGCAAGCTGGAGGACAGGTGAGACAGCAGGACAGGTGAGGCGGGCCCTG
AGGGGGGGGCGGGTGGGAGCCAGGTGAATGTACGGCTCTTGGCGGCCGAGGGGGGGGGGGGCAGGAGGAGGC
AGAGGGCGGCGGAGGAGGAGCCCCCCAGCAGCGAGCGGCGAGCAACTGACCGCGGCCTTCTGACCAGGACCG
GAGCAGGGCCCCAAGCCCCCGGGCCTGGTGGGGGACGCGCTTCTTCCCACACTGTGAGCCTCAGCAGCTCCA
GCCAGCGGACCCGACGGCTGAGAGGAGCCCCAGAACCAGGACTGGGGGATTTGGAGCTGGGCAGAGACTTAA
CCCCCACAGCACCGGGAAGCAGCCAACTCCCCTCGCCTCCTTCCCCCTTCGTGGCTTGCGGTCTCTCTTCCC
CGCCTCGGCCCCCAGGAAGTGAAGATGTTACAGCCTGGTCCTCATCCTCCCTCACCCCAAGCTGCTGCTCCT
GGAGAAGCCTGGCCAGGCCCCTCTCAGGCTCCCTGGCAGAGCCTAGAGGAGAAGATGGGGAGCCCCGAGGAT
GACCTGATTGGGATTCCATTCCCGGACCACAGCAGTGAGCTCCTGAGCTGCCTCAATGAGCAGCGCCAGCTG
GGCCACCTATGTGACCTCACCATCCGGACGCAGGGCCTTGAATACCGCACCCACAGGGCTGTGCTAGCTGCC
TGTAGCCACTACTTCAAGAAGCTTTTCACTGAGGGCGGTGGCGGAGCTGTCATGGGGGCCGGGGGTAGCGGG
ACGGCCACTGGGGGAGCAGGGGCCGGTGTGTGTGAGCTGGACTTTGTAGGGCCAGAGGCACTAGGCGCCCTC
CTTGAATTTGCCTATACAGCCACACTGACCACCAGCAGCGCCAACATGCCAGCTGTGCTCCAGGCTGCCCGC
CTGCTGGAGATCCCGTGTGTCATCGCTGCTTGCATGGAGATTCTGCAGGGCAGTGGGCTAGAAGCTCCCAGC
CCGGACGAGGATGACTGTGAGCGAGCCCGCCAGTATCTGGAGGCCTTTGCCACAGCCACGGCCTCTGGAGTT
CCCAATGGTGAAGACAGTCCTCCACAGGTGCCCCTCCCACCACCTCCGCCACCGCCACCTCGGCCTGTTGCC
CGCCGCAGCCGCAAGCCCCGGAAAGCTTTCCTGCAAACCAAGGGGGCCAGAGCAAACCACCTAGTCCCTGAG
GTGCCCACAGTGCCCGCCCATCCCTTGACCTATGAGGAGGAGGAGGTGGCGGGCAGAGTGGGCAGCAGTGGG
GGCAGTGGGCCGGGGGACAGCTACAGCCCTCCCACAGGAACTGCCTCCCCTCCTGAGGGTCCCCAGAGCTAC
GAACCCTATGAGGGTGAGGAAGAAGAAGAGGAGCTGGTATATCCCCCAGCCTATGGGCTGGCGCAGGGTGGC
GGGCCCCCGCTGTCCCCAGAGGAGCTGGGCTCAGATGAGGATGCCATCGATCCTGACCTGATGGCCTACCTA
AGCTCCCTGCACCAGGACAACCTGGCACCAGGCCTGGACAGCCAAGACAAGCTGGTGCGCAAACGCCGCTCC
CAGATGCCTCAGGAGTGCCCTGTCTGCCACAAGATCATCCATGGGGCAGGCAAACTGCCTCGCCACATGAGG
ACCCACACAGGCGAGAAGCCCTTTGCCTGCGAGGTCTGCGGTGTTCGATTCACCAGGAACGACAAGCTGAAG
ATCCACATGCGGAAGCACACGGGAGAGCGCCCCTACTCATGCCCGCACTGCCCAGCCCGCTTCCTGCACAGC
TACGACCTCAAGAACCACATGCACCTGCACACAGGGGACCGGCCCTATGAGTGCCACCTGTGCCACAAGGCT
TTCGCCAAGGAGGACCACCTGCAGCGCCACCTCAAAGGCCAGAACTGCCTGGAGGTGCGCACCCGACGGCGC
CGCAAGGACGATGCACCACCCCACTACCCACCACCCTCTACCGCTGCTGCATCCCCCGCTGGCCTCGACCTC
TCCAATGGCCACCTGGACACCTTCCGCCTCTCTCTAGCTCGATTCTGGGAGCAGTCAGCCCCCACTGGGCCC
CCGGTCTCTACCCCAGGGCCCCCTGATGACGATGAGGAGGAAGGGGCACCCACCACACCCCAGGCTGAAGGT
GCCATGGAGTCCTCTTAAAGAGGGACGAGGGCCAGACTGAAGCAGCACAAGGCCGGGGACACCCATGCCAAG
CAGTGGGAGCACGCAGGACAGACACAGCAGGGGTCTGGGGCACGGAGCCTTGCTGGCATCAGCATCAGCCCT
TCCTCCCAGAGCCCTCATTCCAATTCCAAGCTAAGAAGGTATTGGGGCAGAGGCTCCCCAAATTGGGGTGAT
CCCCCAAGGAGTGATACATATATTGTGTATATATTTACAGCTGTATTGTAAAAGTGGGGTCCCTGTCCCCAG
CTGCTCCTGGGGAGTAGAAGCAATAATGTATTTCTAATTTGTGGGTCCCACTTCGGCTATGCGGGTTTCTAG
GGGGTGGGGGCTTGGGACCAAAGCCTTGCCCCGCCCCTATGCCCCTTGGGGGTTTTGGCTGTGTAAGGGGGT
GAAGGACTGCCCCTCCCTTTCGAGACCCCTCCTTCCTGGTTTCTGTTCCTTTTTCCTGGCAGTGAATTATGC
AAAGGGGGCCGGCAAAGGAAGGGTAGGTGGGGGAAAGCCAGGTGGAAGCTTGAAAGACTGGGGGACTGGGCC
TGTAAGGAAGGAGCCATCCCAGTCCCCCTCCGCCCTGCTCCCGGCGCTGAGTCATGGGGTCGTGGAGAAGGG
GGCGGGGTGGCCTGATTGGCTCGCCTGCCCCTGGGGGCAGTAGAGGGGCCCCGCCCAGCTAGGGGAGCCGCT
CCGTTCCACTCCCCTCCCTAGCCCTCCCTCCCCACGGCCCTGGGCAGGGAATGTCTTGTTCCCGCCGCTCCC
TCCCCGGGGCCAGAGGGCAGGGCGGGCCGGGCGGCGTCCTACCCTCTTCTCCTCCTCCCCATCTCCTCCCCG
CCCAGGTGCGAGCCGGAGCCGCCGCCACCGCTGCCGCCCCTGACTCACGCCGCCCCCGGGCTGGCGCAGCGA
AGGGTGTGGGACAGGGTAAGGGGTTGGAAGAGCCTTGTGGAGAGCGGGCGAGCCGGCGCCATCTGGCGGCCA
TGCTCTGAGTGGGCGAGCGCCCCCCGCGGCCACTGGAGCGAGCTGTCTTCACGCTCCTCATCCACCCCAGCT
GGTGAGCGGCGCCCCCTTGCCAAGGCAGTGGGCACAGAACTTCTCGCTTGGCCGCAGGGGAAGGGGCTGCGG
ACCTGTGGGAAAGTGATCCCCTTCCCAGATCCTTGCCAGCCGGGCTTCCTGTCAGGCAGGGGAGAATAATCC
CCACTCTGCTCTTAGGATTGAATCCACCCCCATTCTGTACATAGCCTCTTCTGTTGGTCTTGTTGAAATCTA
GTTTCAGATTTTTAACTACCCAATTCTGCTGGGGGTGGGGGACACCCCCCCTTCCTCGCTGGGTGCTGGACC
CCTTTTGCAGCCTGGGCTCTGCCTTGCACTATTTCCCCTTCCTGGCCTGACGGCTCCTCCCCCTCCTTAAAA
GGGGCAGGTTCAGGGGCCCGGTGCTCTTCCTCCCTTCCATGCACCCCCATGCCCATTTGCACAGCTGCCCAG
GTACCCCTAACAGTGGGGAGGGGTCACAGGGAGGGGGTAGCGGGACCAGTCCCTGTTATCTATTTAAAAAGT
GATGATGTAATATATTGGGGTGGCGGGGAGATCGGGTTGTCCTGGGCCTCATCTTAGCATTTCAGGTGATGG
GGGGAGCCCAGGGCTGGGGAGACCTGGGGCCCAGCCCCAGAAAGTGGGGACAATGTGGCCTCCCTTCTCCCT
ACTTTCGGCTTTCCCAGTCAGTGCCTTAGGGGGAGAGGCACTCCCCCCCTCCTATTCCCTTCCCCCCACCCC
AACTCCCCCACCTCGGGTGTAAGCGACAGGAAGAAATAATAATAATTTAAGATTC

In some embodiments, the heterologous nucleic acid encoding ThPOK has at least 80 percent, at least 85 percent, at least 90 percent, at least 95 percent, at least 99 percent, or has 100 percent identify to:

AGCAGCGAGCGGCGAGCAACTGACCGCGGCCTTCTGACCAGGACCGGAGCAGGGCCCCAAGCCCCCGGGCCTGGTG
GGGGACGCGCTTCTTCCCACACTGTGAGCCTCAGCAGCTCCAGCCAGCGGACCCGACGGCTGAGAGGAGAAGATGG
GGAGCCCCGAGGATGACCTGATTGGGATTCCATTCCCGGACCACAGCAGTGAGCTCCTGAGCTGCCTCAATGAGCA
GCGCCAGCTGGGCCACCTATGTGACCTCACCATCCGGACGCAGGGCCTTGAATACCGCACCCACAGGGCTGTGCTA
GCTGCCTGTAGCCACTACTTCAAGAAGCTTTTCACTGAGGGCGGTGGCGGAGCTGTCATGGGGGCCGGGGGTAGCG
GGACGGCCACTGGGGGAGCAGGGGCCGGTGTGTGTGAGCTGGACTTTGTAGGGCCAGAGGCACTAGGCGCCCTCCT
TGAATTTGCCTATACAGCCACACTGACCACCAGCAGCGCCAACATGCCAGCTGTGCTCCAGGCTGCCCGCCTGCTG
GAGATCCCGTGTGTCATCGCTGCTTGCATGGAGATTCTGCAGGGCAGTGGGCTAGAAGCTCCCAGCCCGGACGAGG
ATGACTGTGAGCGAGCCCGCCAGTATCTGGAGGCCTTTGCCACAGCCACGGCCTCTGGAGTTCCCAATGGTGAAGA
CAGTCCTCCACAGGTGCCCCTCCCACCACCTCCGCCACCGCCACCTCGGCCTGTTGCCCGCCGCAGCCGCAAGCCC
CGGAAAGCTTTCCTGCAAACCAAGGGGGCCAGAGCAAACCACCTAGTCCCTGAGGTGCCCACAGTGCCCGCCCATC
CCTTGACCTATGAGGAGGAGGAGGTGGCGGGCAGAGTGGGCAGCAGTGGGGGCAGTGGGCCGGGGGACAGCTACAG
CCCTCCCACAGGAACTGCCTCCCCTCCTGAGGGTCCCCAGAGCTACGAACCCTATGAGGGTGAGGAAGAAGAAGAG
GAGCTGGTATATCCCCCAGCCTATGGGCTGGCGCAGGGTGGCGGGCCCCCGCTGTCCCCAGAGGAGCTGGGCTCAG
ATGAGGATGCCATCGATCCTGACCTGATGGCCTACCTAAGCTCCCTGCACCAGGACAACCTGGCACCAGGCCTGGA
CAGCCAAGACAAGCTGGTGCGCAAACGCCGCTCCCAGATGCCTCAGGAGTGCCCTGTCTGCCACAAGATCATCCAT
GGGGCAGGCAAACTGCCTCGCCACATGAGGACCCACACAGGCGAGAAGCCCTTTGCCTGCGAGGTCTGCGGTGTTC
GATTCACCAGGAACGACAAGCTGAAGATCCACATGCGGAAGCACACGGGAGAGCGCCCCTACTCATGCCCGCACTG
CCCAGCCCGCTTCCTGCACAGCTACGACCTCAAGAACCACATGCACCTGCACACAGGGGACCGGCCCTATGAGTGC
CACCTGTGCCACAAGGCTTTCGCCAAGGAGGACCACCTGCAGCGCCACCTCAAAGGCCAGAACTGCCTGGAGGTGC
GCACCCGACGGCGCCGCAAGGACGATGCACCACCCCACTACCCACCACCCTCTACCGCTGCTGCATCCCCCGCTGG
CCTCGACCTCTCCAATGGCCACCTGGACACCTTCCGCCTCTCTCTAGCTCGATTCTGGGAGCAGTCAGCCCCCACT
GGGCCCCCGGTCTCTACCCCAGGGCCCCCTGATGACGATGAGGAGGAAGGGGCACCCACCACACCCCAGGCTGAAG
GTGCCATGGAGTCCTCTTAAAGAGGGACGAGGGCCAGACTGAAGCAGCACAAGGCCGGGGACACCCATGCCAAGCA
GTGGGAGCACGCAGGACAGACACAGCAGGGGTCTGGGGCACGGAGCCTTGCTGGCATCAGCATCAGCCCTTCCTCC
CAGAGCCCTCATTCCAATTCCAAGCTAAGAAGGTATTGGGGCAGAGGCTCCCCAAATTGGGGTGATCCCCCAAGGA
GTGATACATATATTGTGTATATATTTACAGCTGTATTGTAAAAGTGGGGTCCCTGTCCCCAGCTGCTCCTGGGGAG
TAGAAGCAATAATGTATTTCTAATTTGTGGGTCCCACTTCGGCTATGCGGGTTTCTAGGGGGTGGGGGCTTGGGAC
CAAAGCCTTGCCCCGCCCCTATGCCCCTTGGGGGTTTTGGCTGTGTAAGGGGGTGAAGGACTGCCCCTCCCTTTCG
AGACCCCTCCTTCCTGGTTTCTGTTCCTTTTTCCTGGCAGTGAATTATGCAAAGGGGGCCGGCAAAGGAAGGGTAG
GTGGGGGAAAGCCAGGTGGAAGCTTGAAAGACTGGGGGACTGGGCCTGTAAGGAAGGAGCCATCCCAGTCCCCCTC
CGCCCTGCTCCCGGCGCTGAGTCATGGGGTCGTGGAGAAGGGGGCGGGGTGGCCTGATTGGCTCGCCTGCCCCTGG
GGGCAGTAGAGGGGCCCCGCCCAGCTAGGGGAGCCGCTCCGTTCCACTCCCCTCCCTAGCCCTCCCTCCCCACGGC
CCTGGGCAGGGAATGTCTTGTTCCCGCCGCTCCCTCCCCGGGGCCAGAGGGCAGGGCGGGCCGGGCGGCGTCCTAC
CCTCTTCTCCTCCTCCCCATCTCCTCCCCGCCCAGGTGCGAGCCGGAGCCGCCGCCACCGCTGCCGCCCCTGACTC
ACGCCGCCCCCGGGCTGGCGCAGCGAAGGGTGTGGGACAGGGTAAGGGGTTGGAAGAGCCTTGTGGAGAGCGGGCG
AGCCGGCGCCATCTGGCGGCCATGCTCTGAGTGGGCGAGCGCCCCCCGCGGCCACTGGAGCGAGCTGTCTTCACGC
TCCTCATCCACCCCAGCTGGTGAGCGGCGCCCCCTTGCCAAGGCAGTGGGCACAGAACTTCTCGCTTGGCCGCAGG
GGAAGGGGCTGCGGACCTGTGGGAAAGTGATCCCCTTCCCAGATCCTTGCCAGCCGGGCTTCCTGTCAGGCAGGGG
AGAATAATCCCCACTCTGCTCTTAGGATTGAATCCACCCCCATTCTGTACATAGCCTCTTCTGTTGGTCTTGTTGA
AATCTAGTTTCAGATTTTTAACTACCCAATTCTGCTGGGGGTGGGGGACACCCCCCCTTCCTCGCTGGGTGCTGGA
CCCCTTTTGCAGCCTGGGCTCTGCCTTGCACTATTTCCCCTTCCTGGCCTGACGGCTCCTCCCCCTCCTTAAAAGG
GGCAGGTTCAGGGGCCCGGTGCTCTTCCTCCCTTCCATGCACCCCCATGCCCATTTGCACAGCTGCCCAGGTACCC
CTAACAGTGGGGAGGGGTCACAGGGAGGGGGTAGCGGGACCAGTCCCTGTTATCTATTTAAAAAGTGATGATGTAA
TATATTGGGGTGGCGGGGAGATCGGGTTGTCCTGGGCCTCATCTTAGCATTTCAGGTGATGGGGGGAGCCCAGGGC
TGGGGAGACCTGGGGCCCAGCCCCAGAAAGTGGGGACAATGTGGCCTCCCTTCTCCCTACTTTCGGCTTTCCCAGT
CAGTGCCTTAGGGGGAGAGGCACTCCCCCCCTCCTATTCCCTTCCCCCCACCCCAACTCCCCCACCTCGGGTGTAA
GCGACAGGAAGAAATAATAATAATTTAAGATTCA

In some embodiments, the heterologous nucleic acid encoding ThPOK has at least 80 percent, at least 85 percent, at least 90 percent, at least 95 percent, at least 99 percent, or has 100 percent identify to:

AGCTGGAGGACAGGTGAGACAGCAGGACAGGACCGGAGCAGGGCCCCAAGCCCCCGGGCCTGGTGGGGGACGCG
CTTCTTCCCACACTGTGAGCCTCAGCAGCTCCAGCCAGCGGACCCGACGGCTGAGAGGAGAAGATGGGGAGCCC
CGAGGATGACCTGATTGGGATTCCATTCCCGGACCACAGCAGTGAGCTCCTGAGCTGCCTCAATGAGCAGCGCC
AGCTGGGCCACCTATGTGACCTCACCATCCGGACGCAGGGCCTTGAATACCGCACCCACAGGGCTGTGCTAGCT
GCCTGTAGCCACTACTTCAAGAAGCTTTTCACTGAGGGCGGTGGCGGAGCTGTCATGGGGGCCGGGGGTAGCGG
GACGGCCACTGGGGGAGCAGGGGCCGGTGTGTGTGAGCTGGACTTTGTAGGGCCAGAGGCACTAGGCGCCCTCC
TTGAATTTGCCTATACAGCCACACTGACCACCAGCAGCGCCAACATGCCAGCTGTGCTCCAGGCTGCCCGCCTG
CTGGAGATCCCGTGTGTCATCGCTGCTTGCATGGAGATTCTGCAGGGCAGTGGGCTAGAAGCTCCCAGCCCGGA
CGAGGATGACTGTGAGCGAGCCCGCCAGTATCTGGAGGCCTTTGCCACAGCCACGGCCTCTGGAGTTCCCAATG
GTGAAGACAGTCCTCCACAGGTGCCCCTCCCACCACCTCCGCCACCGCCACCTCGGCCTGTTGCCCGCCGCAGC
CGCAAGCCCCGGAAAGCTTTCCTGCAAACCAAGGGGGCCAGAGCAAACCACCTAGTCCCTGAGGTGCCCACAGT
GCCCGCCCATCCCTTGACCTATGAGGAGGAGGAGGTGGCGGGCAGAGTGGGCAGCAGTGGGGGCAGTGGGCCGG
GGGACAGCTACAGCCCTCCCACAGGAACTGCCTCCCCTCCTGAGGGTCCCCAGAGCTACGAACCCTATGAGGGT
GAGGAAGAAGAAGAGGAGCTGGTATATCCCCCAGCCTATGGGCTGGCGCAGGGTGGCGGGCCCCCGCTGTCCCC
AGAGGAGCTGGGCTCAGATGAGGATGCCATCGATCCTGACCTGATGGCCTACCTAAGCTCCCTGCACCAGGACA
ACCTGGCACCAGGCCTGGACAGCCAAGACAAGCTGGTGCGCAAACGCCGCTCCCAGATGCCTCAGGAGTGCCCT
GTCTGCCACAAGATCATCCATGGGGCAGGCAAACTGCCTCGCCACATGAGGACCCACACAGGCGAGAAGCCCTT
TGCCTGCGAGGTCTGCGGTGTTCGATTCACCAGGAACGACAAGCTGAAGATCCACATGCGGAAGCACACGGGAG
AGCGCCCCTACTCATGCCCGCACTGCCCAGCCCGCTTCCTGCACAGCTACGACCTCAAGAACCACATGCACCTG
CACACAGGGGACCGGCCCTATGAGTGCCACCTGTGCCACAAGGCTTTCGCCAAGGAGGACCACCTGCAGCGCCA
CCTCAAAGGCCAGAACTGCCTGGAGGTGCGCACCCGACGGCGCCGCAAGGACGATGCACCACCCCACTACCCAC
CACCCTCTACCGCTGCTGCATCCCCCGCTGGCCTCGACCTCTCCAATGGCCACCTGGACACCTTCCGCCTCTCT
CTAGCTCGATTCTGGGAGCAGTCAGCCCCCACTGGGCCCCCGGTCTCTACCCCAGGGCCCCCTGATGACGATGA
GGAGGAAGGGGCACCCACCACACCCCAGGCTGAAGGTGCCATGGAGTCCTCTTAAAGAGGGACGAGGGCCAGAC
TGAAGCAGCACAAGGCCGGGGACACCCATGCCAAGCAGTGGGAGCACGCAGGACAGACACAGCAGGGGTCTGGG
GCACGGAGCCTTGCTGGCATCAGCATCAGCCCTTCCTCCCAGAGCCCTCATTCCAATTCCAAGCTAAGAAGGTA
TTGGGGCAGAGGCTCCCCAAATTGGGGTGATCCCCCAAGGAGTGATACATATATTGTGTATATATTTACAGCTG
TATTGTAAAAGTGGGGTCCCTGTCCCCAGCTGCTCCTGGGGAGTAGAAGCAATAATGTATTTCTAATTTGTGGG
TCCCACTTCGGCTATGCGGGTTTCTAGGGGGTGGGGGCTTGGGACCAAAGCCTTGCCCCGCCCCTATGCCCCTT
GGGGGTTTTGGCTGTGTAAGGGGGTGAAGGACTGCCCCTCCCTTTCGAGACCCCTCCTTCCTGGTTTCTGTTCC
TTTTTCCTGGCAGTGAATTATGCAAAGGGGGCCGGCAAAGGAAGGGTAGGTGGGGGAAAGCCAGGTGGAAGCTT
GAAAGACTGGGGGACTGGGCCTGTAAGGAAGGAGCCATCCCAGTCCCCCTCCGCCCTGCTCCCGGCGCTGAGTC
ATGGGGTCGTGGAGAAGGGGGCGGGGTGGCCTGATTGGCTCGCCTGCCCCTGGGGGCAGTAGAGGGGCCCCGCC
CAGCTAGGGGAGCCGCTCCGTTCCACTCCCCTCCCTAGCCCTCCCTCCCCACGGCCCTGGGCAGGGAATGTCTT
GTTCCCGCCGCTCCCTCCCCGGGGCCAGAGGGCAGGGCGGGCCGGGCGGCGTCCTACCCTCTTCTCCTCCTCCC
CATCTCCTCCCCGCCCAGGTGCGAGCCGGAGCCGCCGCCACCGCTGCCGCCCCTGACTCACGCCGCCCCCGGGC
TGGCGCAGCGAAGGGTGTGGGACAGGGTAAGGGGTTGGAAGAGCCTTGTGGAGAGCGGGCGAGCCGGCGCCATC
TGGCGGCCATGCTCTGAGTGGGCGAGCGCCCCCCGCGGCCACTGGAGCGAGCTGTCTTCACGCTCCTCATCCAC
CCCAGCTGGTGAGCGGCGCCCCCTTGCCAAGGCAGTGGGCACAGAACTTCTCGCTTGGCCGCAGGGGAAGGGGC
TGCGGACCTGTGGGAAAGTGATCCCCTTCCCAGATCCTTGCCAGCCGGGCTTCCTGTCAGGCAGGGGAGAATAA
TCCCCACTCTGCTCTTAGGATTGAATCCACCCCCATTCTGTACATAGCCTCTTCTGTTGGTCTTGTTGAAATCT
AGTTTCAGATTTTTAACTACCCAATTCTGCTGGGGGTGGGGGACACCCCCCCTTCCTCGCTGGGTGCTGGACCC
CTTTTGCAGCCTGGGCTCTGCCTTGCACTATTTCCCCTTCCTGGCCTGACGGCTCCTCCCCCTCCTTAAAAGGG
GCAGGTTCAGGGGCCCGGTGCTCTTCCTCCCTTCCATGCACCCCCATGCCCATTTGCACAGCTGCCCAGGTACC
CCTAACAGTGGGGAGGGGTCACAGGGAGGGGGTAGCGGGACCAGTCCCTGTTATCTATTTAAAAAGTGATGATG
TAATATATTGGGGTGGCGGGGAGATCGGGTTGTCCTGGGCCTCATCTTAGCATTTCAGGTGATGGGGGGAGCCC
AGGGCTGGGGAGACCTGGGGCCCAGCCCCAGAAAGTGGGGACAATGTGGCCTCCCTTCTCCCTACTTTCGGCTT
TCCCAGTCAGTGCCTTAGGGGGAGAGGCACTCCCCCCCTCCTATTCCCTTCCCCCCACCCCAACTCCCCCACCT
CGGGTGTAAGCGACAGGAAGAAATAATAATAATTTAAGATTC

Sequences encoding commitment factors are available at GenBank and other well-known gene databases such as Ensembl. Expression of one or more of these factors can help commit the stem or progenitor T cells to the CD4+ cell fate during differentiation. As described above, in some embodiments, the engineered cell comprises one or more heterologous nucleic acids encoding multiple commitment factors. For example, in some embodiments, the engineered cell comprises a heterologous nucleic acid that encodes the Treg lineage commitment factor FOXP3 and/or the lineage commitment factor ThPOK. In some embodiments, the heterologous nucleic acid encodes Helios, which can be expressed in CD4+ T cells to produce a subpopulation of Tregs. In some embodiments, the stem or progenitor T cells may be engineered to overexpress commitment factors that enhance hematopoietic stem cell (HSC) multipotency. In some embodiments, the stem or progenitor cells may be engineered to downregulate expression of certain lineage commitment factors. For example, in some embodiments, the cell can be engineered to downregulate the expression of EZHI, Runx1, or Runx3, via an engineered site-specific transcriptional repression construct (e.g. ZFP-KRAB, CRISPRi, etc.), shRNA, or siRNA.

In some embodiments, this disclosure provides an engineered cell comprising at least one heterologous nucleic acid encoding one or more commitment factors, wherein the one or more commitment factors can be overexpressed, i.e., expressed at a level higher than that which is found in a comparable wild-type cell. In some embodiments, the one or more commitment factors are expressed at least 0.5-fold, at least 1-fold, at least 2-fold, at least 3-fold, at least 4-fold, at least 5-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, at least 95-fold, at least 100-fold, at least 150-fold, at least 200-fold, or at least 250-fold greater than that found in a comparable wild-type cell. In some embodiments, the one or more commitment factors are expressed at least 2-fold greater than that found in a comparable wild-type cell. In some embodiments, the one or more commitment factors as expressed at a level that is at least 5 percent, at least 10 percent, at least 15 percent, at least 20 percent, at least 25 percent, at least 30 percent, at least 35 percent, at least 40 percent, at least 45 percent, at least 50 percent, at least 55 percent, at least 60 percent, at least 65 percent, at least 70 percent, at least 75 percent, at least 80 percent, at least 85 percent, at least 90 percent, at least 95 percent, at least 100 percent, at least 125 percent, at least 150 percent, at least 175 percent, at least 200 percent, or higher with respect to the expression of a substantially identical commitment factor present in a comparable wild-type cell.

In some embodiments, the one or more commitment factors are regulated by an inducible promoter. For example, in some embodiments, the heterologous nucleic acid comprises an inducible promoter, e.g., a tet-inducible promoter, upstream of the nucleotide sequence encoding the one or more commitment factors. Inducible expression of the commitment factors is useful since certain factors may be toxic during some stages of development, e.g., mesodermal, hematopoietic, or lymphocyte developmental stages, and therefore, turning on the commitment factors only during specific T cell development to skew differentiation towards CD4+ T cell lineage is advantageous.

In some embodiments, expression of the commitment factor from the heterologous nucleic acid can be controlled by a doxycycline-inducible promoter. The doxycycline-inducible promoter may include a 5-mer repeat of the Tet-responsive element. Upon the introduction of doxycycline to tissue culture, a constitutively expressed inducible form of the reverse tetracycline-controlled transactivator (rtTA) binds to the Tet-responsive element and initiate transcription of the one or more commitment factor. In some embodiments, the rtTA protein is expressed from a STEL, which ensures stable expression during cell culture and differentiation.

In some embodiments, expression of the one or more commitment factors are regulated by a cell lineage or tissue specific gene promoter. For example, in some embodiments, the one or more commitment factors are regulated by a promoter associated with a gene that is turned on during T cell development. For example, the commitment factor can be positioned on the heterologous nucleic acid downstream of a CD4 gene promoter, a GATA3 gene promoter, a TRAC promoter, or a ThPOK gene promoter.

In some embodiments, expression of the one or more lineage commitment factors can be used to facilitate cellular differentiation along a lineage commitment pathway. For example, in some embodiments, the one or more commitment factors are activated at discrete developmental stages along a lineage commitment pathway to steer differentiation of the engineered cell into a CD4+ T cell. For example, in some embodiments, the commitment factor is ThPOK, and ThPOK is activated when the engineered cell reaches a CD4+CD8+ phenotype, thereby driving the engineered cell towards a CD4+ T cell (i.e., a CD4+CD8− T cell) fate. In some embodiments, the CD4+ T cell can be further differentiated into a CD4+Treg, e.g., by the induction of a second lineage commitment factor, such as FOXP3 and/or Helios.

Accordingly, in some embodiments, this disclosure provides cells that are engineered with multiple, e.g., at least two or at least three, commitment factors. In some embodiments, the multiple commitment factors can be activated to facilitate or promote differentiation of stem or progenitor cells into CD4+ cells. In some embodiments, the multiple commitment factors can be activated to facilitate or promote trans-differentiation of a cell into a CD4+ T cell. In some embodiments, the multiple commitment factors can be used to steer the development of the engineered cell along a differentiation pathway comprising multiple bifurcations or developmental stages. For example, in some embodiments, the engineered cell comprises a first commitment factor that can be activated to steer differentiation towards a first desired cell type at a first bifurcation or developmental stage and a second commitment factor that can be activated to steer differentiation towards a second desired cell type at a second bifurcation or developmental stage. For example, in some embodiments, the engineered cells comprise a first commitment factor that facilitates differentiation of the engineered cell into a CD4+ T cell, and a second commitment factor that facilitates differentiation of the CD4+ T cell into a Treg. For example, the first commitment factor can be ThPOK, and the second commitment factor can be FOXP3. In some embodiments, the multiple commitment factors are regulated by different regulatory elements, e.g., inducible or cell type specifically-active promoters, and as such, can be differentially regulated during differentiation. For example, a first commitment factor may be regulated by a first inducible promoter and a second commitment factor can be regulated by a second inducible promoter that is distinct from the first inducible promoter.

### B. Integration Sites for Exogenous Nucleic Acids

To engineer a cell (e.g., an iPSC), a heterologous nucleic acid encoding a transgene of interest can be introduced into the cell. The heterologous nucleic acid, sometimes referred to as a transgene or an exogenous nucleic acid, can be a contiguous sequence of nucleic acids that is integrated into a site of the genome of the cell where the sequence does not naturally occur. Exemplary transgenes include CD4+ T cell lineage commitment factors or Treg lineage commitment factors some of which are described above. Other transgenes can be, for example, nucleic acids that code for transcriptional activator proteins (e.g., rtTA), which can be used to activate lineage commitment factors, for example, upon addition of a compound (e.g., dox) to cell media. In other instances, heterologous nucleic acids can include antigen recognition receptors, e.g., a TCR or CAR.

### 1. STEL

To date, heterologous nucleic acids (e.g., transgenes) are most commonly targeted to safe harbor sites in the genome such as the AAVS1 locus. High level transgene expression from safe harbor loci typically requires inclusion of external promoter sequences. But different promoters vary in their ability to maintain transgene expression in specific cell populations. Increasing evidence suggests that transgene expression at AAVS1 and other safe harbor sites is not supported in some cell lineages (e.g., dopaminergic neurons, microglia, macrophages, or T cells) and may be subject to promoter silencing. It has been observed that genetically modified human pluripotent stem cells lose transgene expression upon lineage-directed differentiation (see, e.g., Klatt et al., Hum Gene Ther. (2020) 31(3-4):199-210; Ordovas et al., Stem Cell Rep. (2015) 5:918-31, each of which is incorporated by reference). The present disclosure provides methods of transgene expression that circumvent this problem and thus greatly facilitate development of CD4+ T cells and derivatives thereof.

In particular, aspects of the present disclosure provides methods of obtaining genetically modified cells in which an exogenously introduced transgene (e.g., lineage commitment factor) can be induced or can be expressed at a stable, sustained level over a period of time or as the cells differentiate. These methods are especially advantageous when applied to PSCs engineered for use in cell therapy. Genetically modified PSCs obtained by the present methods do not lose transgene expression over time in culture and/or as the cells are differentiated into one or more cells. The transgenes can also be activated at various points during cell differentiation.

In some embodiments, the expression level of the transgene in the modified cells does not change by more than 50%, more than 40%, more than 35%, more than 30%, more than 25%, more than 20%, more than 15%, more than 10%, or more than 5% over one or more cell culture passages, as compared to the expression level of the transgene prior to the one or more passages. The one or more passages may be, e.g., two or more, three or more, four or more, five or more, six or more, seven or more, eight or more, nine or more, ten or more, or 15 or more passages.

In some embodiments, the expression level of the transgene in the modified cells does not change by more than 50%, more than 40%, more than 35%, more than 30%, more than 25%, more than 20%, more than 15%, more than 10%, or more than 5% as the cell state changes in the cells, as compared to the expression level of the transgene prior to the cell state change. A cell state may be, e.g., a cell's pluripotency, biological activity, phenotype, or differentiation status.

The expression level of a gene (e.g., a transgene or an endogenous gene) can be determined by any method suitable for the particular gene. For example, the level of RNA (e.g., by RT-PCR) or protein (e.g., by FRET, ELISA, cytometric analysis, and western blot) expressed from the gene can be measured

In particular, it is an insight of the disclosure that lineage commitment factors, or transcriptional regulators of said lineage commitment factors, can be integrated into certain loci, such as, for example, “sustained transgene expression loci” or abbreviated herein as “STEL”, which are more resistant to silencing than some non-STEL loci. Resistance to silencing may be observed, for example, by the integration of a reporter (e.g., GFP) and the detection of the reporter as the STEL-engineered cells are cultured over time (e.g., over days in culture, optionally including one or more cell passages) or as the cell fate changes (e.g., differentiation from pluripotent stem cells to lineage-specific cells). When the nucleic acid encoding one or more commitment factors is inserted into such a locus, expression of the commitment factor can be sustained, or otherwise rapidly induced, making transgene-dependent steering of cell fate choices much more efficacious. Accordingly, in some embodiments, the heterologous nucleic acid encoding one or more commitment factors, or transcription activators, is integrated into a STEL of the genome of a target cell (e.g., an iPSC).

The STEL of the present disclosure includes, without limitation, certain housekeeping genes that are active in multiple cell types such as those involved in gene expression (e.g., transcription factors and histones), cellular metabolism (e.g., GAPDH and NADH dehydrogenase), or cellular structures (e.g., actin), or those that encode ribosomal proteins (e.g., large or small ribosomal subunits, such as RPL13A, RPLP0 and RPL7). These proteins include those that form ribonucleoprotein complex, focal adhesion, cell-substrate adherens junction, cell-substrate junction, cell anchoring, extracellular exosome, extracellular vesicle, intracellular organelle, or anchoring junction. Some of the proteins are involved in RNA binding, nucleic acid binding (e.g., rRNA or mRNA binding), or protein binding.

In some embodiments, a STEL site is the locus of an endogenous gene that is robustly and consistently expressed in the pluripotent state as well as during differentiation (e.g., as examined by single-cell RNA sequencing (scRNAseq) analysis). For example, the expression level of the endogenous gene does not change (e.g., decrease) by more than 50%, more than 40%, more than 35%, more than 30%, more than 25%, more than 20%, more than 15%, more than 10%, or more than 5% over five or more, ten or more, or 15 or more passages or as the cell state changes (e.g., state of pluripotency and/or differentiation).

In some embodiments, the STEL is a ribosomal protein gene locus, such as an RPL or RPS gene locus. Examples of RPL genes are RPL10, RPL13, RPS18, RPL3, RPLP1, RPL13A, RPL15, RPL41, RPL11, RPL32, RPL18A, RPL19, RPL28, RPL29, RPL9, RPL8, RPL6, RPL18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS16, RPS25, RPS15, RPS20, and RPS11.

In some embodiments, the STEL is a gene locus encoding a mitochondria protein. Examples of such gene loci are MT-CO1, MT-CO2, MT-ND4, MT-ND1, and MT-ND2.

In some embodiments, the STEL is a gene locus encoding an actin protein, such as ACTG1 and ACTB.

In some embodiments, the STEL is a gene locus encoding a eukaryotic translation elongation factor, such as EEF1A1 and EEF2, or a eukaryotic translation initiation factor such as EIF1.

In some embodiments, the STEL is a gene locus encoding a histone, such as H3F3A and H3F3B.

In other embodiments, the STEL is a gene locus selected from FTL, FTH1, TPT1, TMSB10, GAPDH, PTMA, GNB2L1, NACA, YBX1, NPM1, FAU, UBA52, HSP90AB1, MYL6, SERF2, and SRP14.

To introduce a transgene construct into a host cell, one can use a chemical method (e.g., calcium phosphate transfection or lipofection), a non-chemical method (e.g., electroporation or nucleofection), a particle-based method (e.g., magnetofection), or viral delivery (e.g., by using viral vectors such as lentiviral vectors, adeno-associated viral (AAV) vectors, retroviral vectors, and hybrid viral vectors). The transgene may be integrated into the STEL site in a site-specific manner through, for example, a single- or double-stranded DNA break caused by ZFN, TALEN, CRISPR-cas9, CRISPR/cpf1, or another nuclease. For example, one can use various types of homologous recombination gene editing systems, where edited alleles are generated by homologous recombination between the host genome and donor molecules such as double-stranded DNA or single stranded DNA. Homologous recombination may be facilitated by the induction of double-stranded DNA breaks at targeted, homologous loci in the host genome and results in the exchange of the exogenous DNA donor sequence with the endogenous host genomic sequence. See, e.g., Hoshijima et al., Methods Cell Biol. (2016) 135:121-47. However, double-stranded DNA breaks are not required for homologous recombination.

Other well-known gene editing systems may also be used, such as those utilizing genome-targeting elements including a DNA-binding domain (e.g., zinc finger DNA-binding protein or a TALE DNA-binding domain), guide RNA elements (e.g., CRISPR guide RNA), and guide DNA elements (e.g., NgAgo guide DNA). Programmable gene-targeting and nuclease elements enable precise genome editing by introducing DNA breaks, such as double-stranded or single stranded breaks at specific genomic loci. In some embodiments, the genome editing system is a meganuclease based system, a zinc finger nuclease (ZFN) based system, a Transcription Activator-Like Effector-based Nuclease (TALEN) based system, a CRISPR-based system, or NgAgo-based system. In some embodiments, exogenously introduced DNA can be used to harness cellular repair mechanisms to introduce a transgene into the genome via homologous recombination.

In particular embodiments, the genome editing system is a CRISPR-based system. The CRISPR-based system comprises one or more guide RNA elements and one or more RNA-guided nucleases.

In further embodiments, the CRISPR-based system is a CRISPR-Cas system. The “CRISPR-Cas system” comprises: (a) at least one guide RNA element or a nucleic acid comprising a nucleotide sequence(s) encoding the guide RNA element, the guide RNA element includes a nucleotide sequence substantially complementary to a nucleotide sequence at the one or more target genomic regions, and an activator RNA that includes a nucleotide sequence that is capable of hybridizing with the guide RNA; and (b) a Cas protein element comprising a Cas protein or a nucleic acid comprising a nucleotide sequence encoding the Cas protein. The guide RNA and activator RNA can be separate or fused together into a single RNA.

In some embodiments, the CRISPR-based system includes Class 1 CRISPR and/or Class 2 CRISPR systems. Class 1 systems employ several Cas proteins together with a CRISPR RNA (crRNA) as the targeted RNA to build a functional endonuclease. Class 2 CRISPR systems employ a single Cas protein and a crRNA as the targeted RNA. Class 2 CRISPR systems, including the type II Cas9-based system, comprise a single Cas protein to mediate cleavage rather than the multi-subunit complex employed by Class 1 systems. The CRISPR-based system also includes Class 2, Type V CRISPR system employing a Cpf1 protein and a crRNA as the targeter RNA.

The Cas protein is a CRISPR-associated (Cas) double-stranded DNA nuclease. In some embodiments, CRISPR-Cas system comprises a Cas9 protein. In some embodiments, the Cas9 protein is SaCas9, SpCas9, SpCas9n, Cas9-H1F, Cas9-H840A, FokI-dCas9, or D10A nickase. The term “Cas protein,” such as Cas9 protein, includes wildtype Cas protein or functional derivatives thereof (such as truncated versions or variants of the wildtype Cas protein with a nuclease activity).

In some embodiments, the CRISPR-based system is a CRISPR-Cpf system. The “CRISPR-Cpf system” comprises: (a) at least one guide RNA element or a nucleic acid comprising a nucleotide sequence(s) encoding the guide RNA element, the guide RNA comprising a targeted RNA having a nucleotide sequence complementary to a nucleotide sequence at a locus of the target nucleic acid; and (b) a Cpf protein (e.g., cpf1) element or a nucleic acid comprising a nucleotide sequence encoding the Cpf protein element.

The transgene may be transcribed together with the endogenous gene at the STEL site, under the transcriptional control of the endogenous promoter, into one mRNA, and then the RNA sequence for each gene is translated separately through the use of an internal ribosome entry site (IRES) in the mRNA. In yet another approach, the transgene may be inserted in frame into the endogenous gene, e.g., at the 3′ end of the endogenous gene, but separated from the endogenous gene sequence by the coding sequence for a self-cleaving 2A peptide, which causes ribosomal skipping during translation. This arrangement results in production of two separate polypeptides—the payload encoded by the transgene and the polypeptide encoded by the endogenous gene. Examples of self-cleaving peptides are 2A peptides, which are viral derived peptides with a typical length of 18-22 amino acids. 2A peptides include T2A, P2A, E2A, F2A, and PQR (Lo et al., Cell Reports (2015) 13:2634-2644). By way of example, P2A is a peptide of 19 amino acids; after the cleavage, a few amino acid residues from the P2A are left on the upstream gene and a proline is left at the beginning of the second gene. See also the Examples below for the use of a PQR peptide. In other embodiments, the STEL gene and the transgene are transcribed into a single mRNA and expressed as a fusion protein.

In some embodiments, the transgene construct may introduce additional regulatory sequences, such as a transcription termination sequence (e.g., polyadenylation (polyA) site such as a SV40 polyA site) and a sequence that enhances gene expression or RNA stability (e.g., a WPRE element), to the targeted locus. To further ensure sustained expression of the transgene, suitable transcription regulatory elements also may be introduced via the transgene construct into the targeted STEL site. Such elements include, without limitation, a ubiquitous chromatin opening element (UCOE) placed upstream of the promoter, and chromatin insulators that create functional boundaries. Chromatin insulators (e.g., chicken beta globin gene cluster (cHS4) and ArsI) can be enhancer blocking or barrier insulators that prevent silencing heterochromatin from spreading into the transgene.

For additional information of STEL refer to co-owned, international application No. PCT/US2020/055158, which is published as WO2021072329A1, and is incorporated by reference.

### 2. STAPLR

In some embodiments, this disclosure makes use of certain intergenic regions in the mammalian genome that allow consistent levels of expression of transgenes integrated therein, or enabling reliable induction of said transgenes, even as the cell undergoes changes in its differentiation state and/or regardless of cell type. This expands the repertoire of genomic safe harbors where transgenes can be stably integrated and their expression can be maintained over multiple passages and as the cell changes its phenotype. This disclosure thus solves a long-standing problem in transgene expression, for example, in the context of making T cell therapies. These intergenic regions are termed “sustained transcriptionally active payload region” (STAPLR) herein, where payload or genomic payload refers to exogenous or heterologous nucleotide sequences introduced to the region.

As used herein, an intergenic region is a stretch of nucleotide sequences located between two neighboring genes. When referencing an intergenic region, the entire region does not need to be utilized; nor does the present disclosure always refer to the entire intergenic region when using the term intergenic region. For instance, the traditional sense of intergenic region is that there are two genes which are separated by a series of nucleotides; that is, two genes flank the intergenic region. However, as gene databases are being updated, not all adjacent genes are consistently shown to be demarcated by an intergenic region. There are a continuing number of overlapping genes being reported, where an overlapping gene is a series of nucleotides encoding one gene partially intertwines, or overlaps on the same or opposing DNA strand, with a series of nucleotides which encode another gene. With this understanding, there are situations where an intergenic region can include a fragment of a particularly described transcript of an overlapping gene in a particular database or with a different cell line. Intergenic regions are known to contain functionally important elements, such as promoters or enhancers, yet not all intergenic regions comprise such elements. Nearby genes may be thousands of base pairs from the functional area of the intergenic region or can be immediately adjacent to the intergenic region. Additionally, the intergenic region can be of various size. For example, the intergenic region can be at least 100 base pairs in length. In other embodiments, the intergenic region can be at least 150 base pairs in length. In other embodiments, the intergenic region can be at least 200 base pairs in length. In some embodiments, the intergenic region is at least 300 base pairs, at least 400 base pairs, at least 500 base pairs, at least 1000 base pairs, at least 1500 base pairs, at least 2000 base pairs, at least 2500 base pairs, at least 3000 base pairs, at least 3500 base pairs, at least 5000 base pairs, at least 10000 base pairs, at least 15000 base pairs, at least 20000 base pairs, at least 30000, at least 40000, at least 50000, at least 75000, or at least 100000 base pairs in length.

STAPLRs herein comprise open chromatin landscape for landing genomic payloads. In some instances, the STAPLRs are in the vicinity of transcriptionally active genes.

STAPLRs of the present disclosure include, without limitation, the intergenic region between the RPL34 gene (Gene ID: 6164) and the OSTC gene (Gene ID: 58505), the intergenic region between the ACTB gene (Gene ID: 60) and the FSCN1 gene (Gene ID: 6624), the intergenic region between the AK1RIN1 gene (Gene ID: 79647) and the NDUFS5 gene (Gene ID: 4725), the intergenic region between the PRDX1 gene (Gene ID: 5052) and the AKR1A1 gene (Gene ID: 10327), the intergenic region between the PTGES3 gene (Gene ID: 10728) and the NACA gene (Gene ID: 4666), the intergenic region between the MLF2 gene (Gene ID: 8079) and the PTMS gene (Gene ID: 5763), the intergenic region between the RAB13 gene (Gene ID: 5872) and the RPS27 gene (Gene ID: 4840565), the intergenic region between the JTB gene (Gene ID: 10899) and the RAB13 gene (Gene ID: 5872), the intergenic region between the AKR1A1 gene (Gene ID: 10327) and the NASP gene (Gene ID: 4678), the intergenic region between the NDUFS5 gene (Gene ID: 4725) and the MACF1 gene (Gene ID: 23499), the intergenic region between the SRSF9 gene (Gene ID: 8683) and the DYNLL1 gene (Gene ID: 8655), the intergenic region between the MYL6B gene (Gene ID: 140465) and the MYL6 gene (Gene ID: 4637), the intergenic region between the GPX1 gene (Gene ID: 2876) and the RHOA gene (Gene ID: 387), the intergenic region between the HNRNPA2B1 gene (Gene ID: 3181) and the CBX3 gene (Gene ID: 11335), the intergenic region between the ROMO gene (Gene ID: 140823) and the RBM39 gene (Gene ID: 9584), and the intergenic region between the PA2G4 gene (Gene ID: 5036) and the RPL41 gene (Gene ID: 6171). In some embodiments, the genes herein refer to human genes and the mammalian cells are human cells.

In some embodiments, the STAPLR is located in the vicinity of at least one sustained transgene expression loci (STEL) as described above. While a STAPLR can be associated with a STEL site, it does not need to be associated with a STEL site. STEL sites may be identified from single cell RNA sequence data. A defining characteristic of a desirable STEL site is the ubiquity of expression. Initially, the STEL site search was focused on the data available at hand, including PSCs and PSC-derived dopamine neurons (and select progenitor states), microglia (and select progenitor states), and cardiomyocytes (and select cardiomyocyte progenitor states). This wide range in both cell type and state of maturity resulted in the identification of STEL sites. Adding publicly available single cell RNA sequencing data of adult human tissue allows for the refining of such a STEL analysis.

Some exemplary STAPLRs are shown in Table 1.

TABLE 1
Intergenic Regions Between Select Genes
Description

Intergenic region between the RPL34 gene and the OSTC gene
Intergenic region between the ACTB gene and the FSCN1 gene
Intergenic region between the AKIRIN1 gene and the NDUFS5 gene
Intergenic region between the PRDX1 gene and the AKR1Al gene
Intergenic region between the PTGES3 and the NACA gene
Intergenic region between the MLF2 and the PTMS gene
Intergenic region between the RAB13 and the RPS27 gene
Intergenic region between the JTB and the RAB13 gene
Intergenic region between the AKR1A1 and the NASP gene
Intergenic region between the NDUFS5 and the MACF1 gene
Intergenic region between the SRSF9 and the DYNLL1 gene
Intergenic region between the MYL6B and the MYL6 gene
Intergenic region between the GPX1 and the RHOA gene
Intergenic region between the HNRNPA2B1 and the CBX3 gene
Intergenic region between the ROMO and the RBM39 gene
Intergenic region between the PA2G4 and the RPL41 gene

In some embodiments, the exogenous nucleotide sequence has been integrated at a location that is at least 100, at least 200, at least 300, at least 400, at least 500, at least 1000, at least 1500, at least 2000, at least 2500, at least 3000, at least 3500, at least 5000, at least 10000, at least 15000, or at least 20000 base pairs away from the nearest gene.

In some embodiments, the expression of the exogenous nucleotide sequence is under the control of a constitutive promoter. In some embodiments, the expression of the exogenous nucleotide sequence is under the control of a tissue-specific or lineage-specific promoter, for example, a promoter of a gene that is expressed in CD4+ T cells. In some embodiments, the expression of the exogenous nucleotide sequence is under the control of an inducible promoter (e.g., lac operon, which can be triggered by Isopropyl β-D-1-thiogalactopyranoside (IPTG); TRE promoter, which can be bound by a transcriptional activator upon its binding by tetracycline and its derivatives).

In some embodiments, the exogenous nucleotide sequence is a transgene encoding a protein of interest, e.g., a lineage commitment factor, such as, ThPOK. In certain embodiments, the protein of interest is a therapeutic protein (e.g., a protein that can improve or prevent symptoms of a disease or condition). Examples of therapeutic proteins include, without limitation, cytokines or growth factors that influence cell identify or that regulate immunity, recombinant antigen receptors, proteins that regulate differentiation or activity of the modified cells, and the like. In some embodiments, the protein of interest is a cellular marker, a protein used for immune evasion, or a safety or kill switch used in cell therapy.

In some embodiments, integration of the exogenous nucleotide sequence in the STAPLR is achieved by using a genomic editing system selected from the group consisting of a CRISPR/Cas system, a Cre/Lox system, a FLP-FRT system, a TALEN system, a ZFN system, a homing endonuclease, random integration (e.g., through transposons), homologous recombination, and non-nuclease dependent viral vectors (e.g., retroviral, AAV, or lentiviral vectors).

In some embodiments, the CRISPR/Cas system comprises a gRNA (guide RNA)-dependent nuclease (or a coding sequence thereof) targeting a selected intergenic region, a gRNA (or a coding sequence thereof), and a donor DNA comprising the exogenous nucleotide sequence.

In some embodiments, the gRNA-dependent nuclease is selected from the group consisting of Cpf1, Cas1, Cas1B, Cas2, Cas3, Cas4, Cas5, Cas6, Cas7, Cas8, Cas9, Cas12, Cas13, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasX, CasY, Csx3, Csx1, Csx15, Csf1, Csf2, Csf3, CasPhi, MAD7 and Csf4.

In some embodiments, the mammalian cell is a pluripotent stem cell (PSC) such as an induced PSC (iPSC), or an embryonic stem cell. In some embodiments, the cell is a cell derived from a PSC or ESC.

The cells, such as human cells, may be engineered in vitro, in vivo, or ex vivo by gene editing methods such as those described herein. A variety of human cell types may be engineered to express a transgene of interest. In some embodiments, the cells to be engineered are pluripotent stem cells, such as human embryonic stem cells (hESCs) or human induced pluripotent stem cells (iPSCs), which can be subsequently induced to differentiate into a desired cell type, referred to herein as PSC-derivatives, PSC-derivative cells, or PSC-derived cells. In still other embodiments, the cells to be engineered are differentiated cells (e.g., partially or terminally differentiated cells). Partially differentiated cells may be, for example, tissue-specific progenitor or stem cells, such as hematopoietic progenitor or stem cells, skeletal muscle progenitor or stem cells, cardiac progenitor or stem cells, neuronal progenitor or stem cells, and mesenchymal stem cells.

The present disclosure also provides targeting vectors for integrating exogenous nucleotide sequences into the STAPLRs. As used herein, a “targeting vector” is a nucleic acid comprising sequences homologous to endogenous chromosomal nucleotide sequences that flank the desired integration location in the genome. These flanking homology sequences are referred to as “homology arms.” Homology arms direct the targeting vector to a specific chromosomal location within the genome by virtue of the homology existing between the homology arms and the corresponding endogenous nucleotide sequence. In some embodiments, the targeting vector is a nucleic acid comprising a nucleic acid of interest, flanked by a 5′ nucleotide sequence (a left homology arm or homology region) and a 3′ nucleotide sequence (a right homology arm or homology region), wherein the 5′ nucleotide sequence and the 3′ nucleotide sequence are nucleotide sequences in a STAPLR locus in the genome of the cell and mediate integration of the nucleic acid of interest through homology into the STAPLR. The STAPLR may be selected from the group consisting of the intergenic region between the RPL34 gene (Gene ID: 6164) and the OSTC gene (Gene ID: 58505), the intergenic region between the ACTB gene (Gene ID: 60) and the FSCN1 gene (Gene ID: 6624), the intergenic region between the AKIRIN1 gene (Gene ID: 79647) and the NDUFS5 gene (Gene ID: 4725), the intergenic region between the PRDX1 gene (Gene ID: 5052) and the AKR1A1 gene (Gene ID: 10327), the intergenic region between the PTGES3 gene (Gene ID: 10728) and the NACA gene (Gene ID: 4666), the intergenic region between the MLF2 gene (Gene ID: 8079) and the PTMS gene (Gene ID: 5763), the intergenic region between the RAB13 gene (Gene ID: 5872) and the RPS27 gene (Gene ID: 4840565), the intergenic region between the JTB gene (Gene ID: 10899) and the RAB13 gene (Gene ID: 5872), the intergenic region between the AKR1A1 gene (Gene ID: 10327) and the NASP gene (Gene ID: 4678), the intergenic region between the NDUFS5 gene (Gene ID: 4725) and the MACF1 gene (Gene ID: 23499), the intergenic region between the SRSF9 gene (Gene ID: 8683) and the DYNLL1 gene (Gene ID: 8655), the intergenic region between the MYL6B gene (Gene ID: 140465) and the MYL6 gene (Gene ID: 4637), the intergenic region between the GPX1 gene (Gene ID: 2876) and the RHOA gene (Gene ID: 387), the intergenic region between the HNRNPA2B1 gene (Gene ID: 3181) and the CBX3 gene (Gene ID: 11335), the intergenic region between the ROMO gene (Gene ID: 140823) and the RBM39 gene (Gene ID: 9584), the intergenic region between the PA2G4 gene (Gene ID: 5036) and the RPL41 gene (Gene ID: 6171).

In some embodiments, the 5′ nucleotide sequence and the 3′ nucleotide sequence are sufficiently similar to the nucleotide sequences in a STAPLR in the genome of the cell to allow integration of the nucleic acid of interest through homology into the sustained transcriptionally active payload region. The 5′ and 3′ sequences are sufficiently similar to the nucleotide sequences as necessary so that the desired function of the nucleotide sequences remains undisturbed. In some embodiments, function remains undisturbed wherein the nucleotide sequences are at least 80%, at least 85%, at least 90%, at least 91%, at least 92%, at least 93%, at least 94%, at least 95%, at least 96%, at least 97%, at least 98%, or at least 99% identical to nucleotide sequences in the targeted STAPLR.

In some embodiments, the nucleic acid of interest is operably linked to a constitutive promoter. In some embodiments, the nucleic acid of interest is operably linked to a cell-type specific and/or an inducible promoter.

In the methods of the disclosure, the homology arms vary in length. In some embodiments, each of the homology arms is independently about at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, or at least 2000 base pairs long.

In the methods of the disclosure, the homology arms (i.e., the 5′ and 3′ nucleotide sequences) can be designed to target anywhere within the disclosed intergenic region.

In the present genetically modified mammalian cells, the exogenous nucleotide sequence may be integrated in a STAPLR at a location that is, for example, at least 100 (e.g., at least 200, at least 500, at least 1000, at least 2000, at least 3000, at least 4000, or at least 5000) base pairs away from the nearest gene to allow for homology arms of about 50 or more base pairs to incorporate a payload and avoid interference with a neighboring gene, even though there are situations where a portion of a homology arm may overlap with a portion of a neighboring gene without disrupting its function.

The present disclosure also provides methods of identifying STAPLRs as sites for safe genomic integration of lineage commitment factors in a mammalian cell (e.g., a human cell). In these methods, the first step is to select a set of cell types for single cell RNA sequencing (“scRNAseq”). Examples of cell types, without limitation, are those referred to herein, including, without limitation, PSCs (e.g., iPSCs), CD4+ T cells, and derivatives thereof, such as Tregs.

The second step is to perform a scRNAseq assay wherein the sequencing analysis assigns a unique transcriptome comprising transcribed genes to each cell that passes quality criteria. To pass quality criteria, transcriptomes are filtered to exclude those with high sparsity or missingness and those that are likely derived from more than one cell.

Next, a Prevalence Score is assigned to each gene. The Prevalence Score is out of “1” and represents the fraction of cells containing at least one transcript of a given gene based on an scRNAseq database of datasets collected. After assigning a Prevalence Score, the location of each gene in the mammalian (e.g., human) genome is determined.

The next step in identifying a STAPLR in the genome of a mammalian cell is to identify neighboring, nonoverlapping genes. By “non-overlapping genes” it is meant that the genes are separated from each other by at least 100 base pairs, at least 200 base pairs, at least 300 base pairs, at least 400 base pairs, at least 500 base pairs, at least 1000 base pairs, at least 1500 base pairs, at least 2000 base pairs, at least 2500 base pairs, at least 3000 base pairs, 3500 base pairs, at least 5000 base pairs, at least 10000 base pairs, at least 15000 base pairs, or at least 20000 base pairs on either strand. The transcripts used to calculate genetic distances for identifying non-overlapping genes may be specified by any genomic database, such as NCBI's RefSeq database and the GENCODE databases.

In some instances, different genomic databases contain non-consensus gene boundary annotations that may lead to different calculated genetic distances and contrary conclusions as to whether two genes overlap or not. In such instances, two genes are considered non-overlapping if they are determined to be non-overlapping by using at least one genomic database. For example, MLF2 is flanked downstream by its neighboring gene PTMS. As annotated in the NCBI RefSeq database, these genes are non-overlapping, with an intergenic distance of about 13 kb; however, the GENCODE V38 database reports one MLF2 transcript whose transcriptional start site is located within the first intron of PTMS encoded on the opposite strand. In this case, the RefSeq annotations are considered and the GENCODE annotations are not, and this gene pair is classified as non-overlapping.

Once two or more genes are considered non-overlapping, a Neighbor Score for the pairs of non-overlapping genes or for regions comprising three or more non-overlapping genes is determined. A Neighbor Score is the product of the individual Prevalence Scores and reflects the probability of both genes being transcriptionally active in the aggregate scRNAseq dataset. The Neighbor Score is essentially a ranking of the vicinities of transcriptionally active genes.

Neighbor Scores are then sorted to obtain a ranking of pairs of non-overlapping genes or a ranking of regions comprising three or more genes. Intergenic regions with high-ranking Neighbor Scores are then annotated in order to design homology arms for site-specific integration. In general, sequences to be avoided for integration sites include promoter regions, enhancer regions, CpG islands, epigenetic marks (e.g., H3K4Me1, H3K4Me3, and H3K27Ac), DNase I hypersensitivity peaks, conserved regions, and repetitive regions. The UCSC Genome Browser may be used with the following gene annotation tracks: GENCODE V32, RefSeq Genes, GTEx RNA-seq, EPDnew Promoters, ENCODE (transcription, H3K4Me1, H3K4Me3, H3K27Ac, and DNase Clusters), GeneHancer, CpG Islands, Conservation 100 vertebrates, and RepeatMasker.

Once the Neighbor Scores are ranked, a pair of genes or a region comprising three or more genes with the best Neighbor Scores is selected and the intergenic region between the genes of the selected pair or region is identified as a potential STAPLR.

The STAPLR may be targeted for safe genetic integration. In selecting a targetable intergenic subregion, known promoter regions and enhancer regions must be avoided. Additionally, conserved regions, repetitive regions, epigenetic marks, and DNase hypersensitivity regions are features that should be minimized in selecting a targetable region. In some embodiments, the targetable intergenic subregion comprises the sequence of an endonuclease protospacer adjacent motif (PAM) site. A PAM site is a 2-6 base pair DNA sequence immediately following the DNA sequence targeted by a Cas9 endonuclease. A short oligonucleotide known as a guide RNA (gRNA) is synthesized to perform the function of the tracrRNA-crRNA complex in a CRISPR/Cas gene editing system. A gRNA recognizes gene sequences having a PAM sequence at the 3′ end. Different Cas proteins may recognize different PAMs. For example, Cas9 from Streptococcus pyrogenes recognizes 5′-NGG-3′ (“N”: any nucleobase); Cas9 from Staphylococcus aureus recognizes 5′-NNGRR(N)-3′; Cas9 from Neisseria meningitidis recognizes 5′-NNNNGATT-3′; Cas9 from Campylobacter jejuni recognizes 5′-NNNNRYAC-3′ (“Y”: a pyrimidine); Cas9 from Streptococcus thermophilus recognizes 5′-NNAGAAW-3′ (“W”: A or T); Cpf1 (Cas12a) from Lachnospiraceae bacterium and Acidaminococcus sp. recognizes 5′-TTTV-3′ (“V”: G, A, or C); Cas12b from Alicyclobacillus acidiphilus recognizes 5′-TTN-3′; and Cas12b v4 from Bacillus hisashii recognizes 5′-ATTN-3′, 5′-TTTN-3′, and 5′-GTTN-3′.

Some exemplary guide RNAs for integrating heterologous nucleic acids into certain STAPLRs are identified below in Table 2.

TABLE 2
gRNAs for Targeting Select STAPLR Sites

| STAPLR Site | gRNA Sequence (5′→3′) |
|---|---|
| PRDX1-AKIR1A1 | CTTGCAGCACTGCCTAGGCT |
| | TGGTTCTTGCAGCACTGCCT |
| | TCTTGCAGCACTGCCTAGGC |
| ACTB-FSCN1 | GCCCACCTTGAAGATCTGTA |
| | TGCCCACCTTGAAGATCTGT |
| | CACCTTGAAGATCTGTAGGG |
| RPL34-OSTC | TTATCATGATACTATGTCCC |
| | AGTTACTGAGCACAGTGCCT |
| | GAGTTACTGAGCACAGTGCC |
| AKIRIN1-NDUFS5 | ATTAATGCCTTTAAATAGGT |
| | TCTTAAACCTACCTATTTAA |
| | GCTAATTAATGCCTTTAAAT |

A list of additional Cas9- and Cpf1-based gRNAs for STAPLR targeting is listed in Table 3.

TABLE 3
Additional STAPLR-Targeting gRNAs

| STAPLR Site | Nuclease | gRNA Sequence (5′→3′) |
|---|---|---|
| PRDX1-AKIRIA1 | Cas9 | CTTGCAGCACTGCCTAGGCT |
| | | TGGTTCTTGCAGCACTGCCT |
| | | TCTTGCAGCACTGCCTAGGC |
| | | TAGGCTGGGCTCAAACTCCA |
| | | CTAGGCTGGGCTCAAACTC |
| | | TGAGAGGATCACTTGAGCCC |
| | | CTGGAGTTTGAGCCCAGCCT |
| | | TTCTTTGTTTGTTTAGAGAC |
| | Cpf1 | AGCCCAGCCTAGGCAGTGCTG |
| | | GAGACTGGTTCTTGCAGCACT |
| | | TTTAGAGACTGGTTCTTGCAG |
| | | TTTGTTTAGAGACTGGTTCTT |
| | | TTTGTTTGTTTAGAGACTGGT |
| ACTB-FSCN1 | Cas9 | GCCCACCTTGAAGATCTGTA |
| | | TGCCCACCTTGAAGATCTGT |
| | | GGGCCAAGAGGCTCAGTCAC |
| | | GGATAAAGAGATCAGGTCCA |
| | | CACCTTGAAGATCTGTAGGG |
| | | TCCCTACAGATCTTCAAGGT |
| | | TTCAAGGTGGGCAAGGAACT |
| | | TCCCTCCCTACAGATCTTCA |
| | | CTCCCTACAGATCTTCAAGG |
| | | ACCTTGAAGATCTGTAGGGA |
| | | CTTCAAGGTGGGCAAGGAAC |
| | | ACAGATCTTCAAGGTGGGCA |
| | | AAGAGATCAGGTCCAAGGCC |
| | | GGGCAAGGAACTGGGCCAAG |
| | | TAGGGAGGGATAAAGAGATC |
| | | ATCAGGTCCAAGGCCAGGTG |
| | | AGGTCCAAGGCCAGGTGCGG |
| | | TGAACCACCGCACCTGGCCT |
| | Cpf1 | TCCCTCCCTACAGATCTTCAA |
| RPL34-OSTC | Cas9 | TTATCATGATACTATGTCCC |
| | | AGTTACTGAGCACAGTGCCT |
| | | GAGTTACTGAGCACAGTGCC |
| | Cpf1 | CAAGCATTTATCATGATACTA |
| | | TCATGATACTATGTCCCAGGC |
| AKIRIN1-NDUFS5 | Cas9 | ATTAATGCCTTTAAATAGGT |
| | | TCTTAAACCTACCTATTTAA |
| | | CCACTTCCCTCCTCCATTAA |
| | | GCTAATTAATGCCTTTAAAT |
| | | CTTTAATGGAGGAGGGAAGT |
| | | CCTTTAATGGAGGAGGGAAG |
| | | TTTAATGGAGGAGGGAAGTG |
| | Cpf1 | ATGGAGGAGGGAAGTGGGGTG |
| | | AGAGAATCTATGTCACCCCAC |
| | | AAGGCATTAATTAGCGTTTGC |
| | | AATAGGTAGGTTTAAGAGAAT |
| | | CATGCACTACTTTAAAATTTT |
| | | AAGTAGTGCATGCAAACGCTA |

For each STAPLR site, human iPSCs can be nucleofected with each individual gRNA complexed with Cas9 nuclease in the form of a ribonucleoprotein (RNP). Three days later, the nucleofected cells can be harvested, genomic DNA was extracted, and PCR amplification of the genomic region flanking the intended cut site was performed. Purified PCR product can be sequenced and the sequencing data can be analyzed for overall cutting efficiency through Synthego's ICE Analysis Tool (available at Synthego's website). gRNAs can be considered to be efficient when showing greater than 50% indel editing.

The gRNA that have the greatest overall cutting efficiency can be selected for use in future experiments to integrate transgenes at STAPLR sites.

A list of gene neighbors consisting of genes that were both highly expressed was generated. This list was filtered to remove gene pairs that contained at least one gene that is a known tumor suppressor gene or oncogene. Initially, gene pairs with less than 5 kb intergenic distance between them were discounted. However, gene pairs with only about 100 base intergenic distance between flanking genes can also be annotated and tested. Promoter regions, enhancer regions, CpG islands, and regions containing epigenetic markers can be avoided in the design. Subregions that avoided regulatory elements and are capable of being synthesized in a donor plasmid can be classified as potential homology arm regions and were used as the basis for a gRNA search (Table 4).

TABLE 4
Exemplary Parameters for Selecting STAPLRs
STAPLR Site Parameters

Site is flanked by adjacent highly expressed genes
Neither flanking gene is a known tumor suppressor gene or oncogene
>100 base intergenic distance between flanking genes
Site avoids annotated regulatory elements and repetitive regions
Site allows for homology arms to be synthesized
and cloned into donor construct
Site has CRISPR gRNAs available that have predicted high
efficiencies and low number of off-targets

After selecting gRNAs with predicted high efficiency, homology arm sequences can be finalized to center selected gRNAs within an 800 bp left homology arm and an 800 bp right homology arm that flanked the intended site of transgene integration. Table 5 indicates the intergenic distance in base pair between the two gene neighbors for exemplary STAPLR site, along with the coordinates for each set of STAPLR left and right homology arms based on the hg38 human reference genome. Gene distances were calculated using NCBI's RefSeq database.

TABLE 5
Exemplary Intergenic Distance Between STAPLR Gene
Neighbors and STAPLR Homology Arm Coordinates

| | | | Distance | Distance |
|---|---|---|---|---|
| | | | from | from |
| | | | Insertion | Insertion |
| | | | Point to | Point to |
| | Homology Arm | | Upstream | Downstream |
| | Chromosomal | Intergenic | Gene | Gene |
| Gene | Coordinates | Distance | Neighbor | Neighbor |
| Neighbors | (Hg38) | (bp) | (bp) | (bp) |
| RPL34- | chr4:108,638,253- | 20100 | 8568 | 11532 |
| OSTC | 108,639,852 | | | |
| ACTB- | chr7:5,545,025- | 62214 | 15223 | 46991 |
| FSCN1 | 5,546,624 | | | |
| AKIRIN1- | chr1:39,012,798- | 20229 | 7533 | 12696 |
| NDUFS5 | 39,014,397 | | | |
| AKR1A1- | chr1:45,525,540- | 27888 | 24439 | 3449 |
| PRDX1 | 45,527,139 | | | |

Sequences for the left and right homology arms of the targeting constructs based on the Hg38 Human Reference Genome are shown in the table below.

TABLE 6
Exemplary STAPLR Left and Right Homology Arms

| STAPLR Site | Left Homology Arm | Right Homology Arm |
|---|---|---|
| AKIRIN1-NDUFS5 | TTCTCATTTAGCAAGAGTGG | TATTTAAAGGCATTAATTAG |
| | AAAACTGTTGTGGATCCCAG | CGTTTGCATGCACTACTTTA |
| | GAGAAGTCTGGAGCTAGGTA | AAATTTTCCTCTCAATTGCTT |
| | GTAGGGGCTAGATAATAGA | GAGTCCAGGAGTTCGAGGTT |
| | GGGCTTTGACTAATAGGAGT | ACGGTGAACTATGATTTCAC |
| | TTGGGCCTTATCCTGTGGAA | CATTGCAGTCCAGCTGAGGC |
| | ATCTGAAGGGTTTTGGGAAA | AACAGAGAGAGACCCTCTTA |
| | ATGAATTCAGGGCTAGGTGC | AAGAAAAAAAAAACTTTTTC |
| | AAGACAGAAGGGCAAATAC | CTCTCAGACTCTGTCCCTAG |
| | CAGACTAAGACACACAGCC | CATTGACTCACCCCATCATT |
| | ACTAAGAGCCTCTTTTTTTTT | TTTTTTTTTTTTCTTGAGATG |
| | TTTTGTCCAGTCAGAGGTAA | GAGTTTCAAGAAAAAACTCC |
| | TGAGGCCTATATATTTATTA | AGGCTGGAGGCAGTGGTATG |
| | GGAAGGATGATTAACCATGG | GTCTCGGCTCACTGCAACCT |
| | CTGTAGCTTCACCTAAGAGG | CCGCCTCCCGGGTTCAAGCG |
| | AACGTGGCTCTTGGGCCGGG | ATTCTCCCTGCCTCAGCCTCC |
| | TGCGGTGGCTCACGCCTGTA | CAAATAGCTGGGATTACAGG |
| | ATCCCAGCACTTTGGGAGGT | CACCCGCCACCACACCCAGC |
| | AGAGGTGGGCGGATCACCTGAG | TAATTTTTTGTATTTTTAGCAGA |
| | GTCAGGAGTTCAAGACCA | GACGGGGCTTCTCCATAT |
| | GCCTGACCAACATGGAGAA | TAGCCAAGCTGGTCTGGAAC |
| | ACCTCTTCTCTACTAAAAAT | TCCTGACTTCAAGTGATCCA |
| | ACAAAATTAGCCGGGCGTGG | CCCACCTTGGCCTCCCAAAG |
| | TGGTGCATGCCTGTAATCCC | TACTGGGATTACAGGCACGA |
| | TGTTACTCGGGAGGCTGAGG | GCCACCGCGCCCGGCCTTCA |
| | CAGGAGAATCGCTTGAACCT | CCTATTATATCTATTCTTGTC |
| | GGGAGGTGGAGGTTGCGGT | TTTTAAATAGTGAGTGACTT |
| | GAGCTGAGATCACACCATTG | CACAGCTCAGTCTCTTTGTTC |
| | CACTCCAGCCTGGACAACAA | TGAACTTCCATTCTGAGTCG |
| | GAGCAAAACTCTATCTCAAA | CAACCCAGGTACACCTTAGG |
| | AAAAAAGAGAGGAATGTGG | AAGGGCGGGATCCCGTTATC |
| | CTCTTGAAGAATCTTTCTAG | CTGTCCCTGAACATCAACTA |
| | TCTCATCTTCCCTGTCCCCTT | CTGAGACAGCTGATGAAGCT |
| | AGACACACCTTCAAAGATCC | GGCTCGGTGTGTGACGGAGG |
| | TTTTTCTTTTGGTTTGGCTTG | CCCCCTCAGAGTCAGCAGAG |
| | TCTTTCCCCCTCACTTTACCT | CCGAGCGTACAGCGCACATC |
| | AATCCAGCCCAATTCCATAG | AGGCAGAGCAGACCAGAGG |
| | CCACTGCTGGTTCCTTTAAT | CAGGGAAGACACACTGGGG |
| | GGAGGAGGGAAGTGGGGTG | CAGGGCCAGTGGGCACATCT |
| | ACATAGATTCTCTTAAACCT | GGACCACCTGCGAGGG |
| | ACC | |
| RPL34-OSTC | TTATTCTTCACAGAGGACAG | CACAGGCACTGTGCTCAGTA |
| | CTCCCAACTCTTTACCTCTCT | ACTCAAGTCATTATAATACT |
| | TCCTAATGCAACACTCACTC | GTGTGCTTGACAATATGTTG |
| | TCACTCTCAGCAGCCTTATA | TACTTACTGGCCAAAAATCA |
| | ATATCTGTAAATGTAGAAAC | CCAATGTCTAATTGTCAAGC |
| | TATCAGTTATGAACTTCTGT | TAAATGTCTTCTTTCAATTTC |
| | AACTCCCTTTTCATCTTTGGC | AATCTATCTGACATGCTGGA |
| | ACCTCAAAACTTTAATGTAT | AACTTCCCTTTCCAAGGCTT |
| | GTAAAAGCATGGGCTTTGAA | CTTTGCTATCGCTCCCTTGGC |
| | ATTGGTAAACTGGAGCTCTA | TCTTCTGTTCCTTTGATCTTG |
| | TCAATCTCTGGCTGTGTCAA | CTGTATTTACTTTGTAGTATC |
| | CTTGAGCATGTTGCCCAATT | CTCCTAATTATGGGGGTGAT |
| | TCTATGAGCCTCAGTTTTCTT | TCCCTAGGCATCCATCTTCA |
| | ATCTAAAAAATGAAACTAAT | GCTTTCTTCTCTCTCACTCTT |
| | AATGCCTACTCCAAAGAATT | TTAACTTTACTCTGTGAATTT |
| | GTTGGATGGATTAAGTAAGA | CCTGTAATTATACACTACAT |
| | CAACCTAATATAATTGCTGT | TTGGTGTGTGTGTGTGTGTG |
| | GCATAGTAAATCCTAACTGT | TCTGTGTGCACGTGCATTTTTC |
| | TGGTTGCCTTATACCTCTAATC | TAGGAAGGAGTTCATAGCT |
| | TTCTTTCTGCTCTCTTTTCT | TTTATCAGCTCTGCAAGGAG |
| | TTAGAAGATGGCATGCATCT | GTCTATGACCCTCCCAATAA |
| | TTTCCAAGGTAAATTTTACC | CAACCCTGACCAGTTCCACC |
| | TCTGTTGAACTCTATCCCAT | TCACGTGTAGACACATTCTT |
| | ACTCTCTTGCCATGGCCCTA | CCACTGTGATGACTTCCATT |
| | ATTTTGTTCAGTTACTTAACT | ATCCTCCACATGCTGCTGAT |
| | CCTTCCCTTCCCTCTGACATC | TCCTGGATCTCCAACCCTAA |
| | CTTGGTCCCTTCCTCTCCACA | ACCTCTTTCCCATGCTCCAG |
| | GTTTTATTCTCTCGTCTTACAC | CGACCATCTTCCCAACTCTG |
| | AAACATGCCCAAGTCTCAT | CCTAGGTCCCTCCTGGAGGG |
| | CATCCATAAAAACCTCTGTC | TCCTTCTAGAATAATAGTTC |
| | ATTACCACTTCAGCTCTCTTC | TCATCAGGCAGGTGGCAGAA |
| | TTCTGTTCATACCCAGCTGT | CCTCTGTCTCCCACTCCTTGCA |
| | GTTGGGTGCCATGAATCATT | TGCCCCTCTGTGAGGCTTG |
| | CACTACTATTATTTTTAAAAT | GGTGGGTTGGGGGTGATGGG |
| | TCCTATCACTACTTCTCCCCT | AGGGGTGCGTGTATAATCAG |
| | CTGCAATCTGGCTTCTCCTG | AGTCCCTGGGGTGCAGAAGC |
| | CCAACATGCTACTGAAATTA | TCTGTTCTGACCTTGCCTCTT |
| | ATGAATTAATTCATCAAATA | GCTATCCATGGAATTACTGC |
| | TTTACAAGCATTTATCATGATAC | TCTATCCTGGCCATTTTCCCATTC |
| | TATGT | TCTTTCT |
| ACTB-FSCN1 | GAAGCCGGGTGTGGTGGTGC | GTAGGGAGGGATAAAGAGA |
| | ATGCCTGTGGTCCCAGCCAC | TCAGGTCCAAGGCCAGGTGC |
| | TTGGGAAACTGAGGTGGGA | GGTGGTTCACGCCTGTAATC |
| | GGATTGCTCAAGCCCAGGAG | CCAGCACTTTGGGAGGCCGA |
| | GTCAAGGCTGCTGTGAGCTA | GCCAGGCAGATCGCGAGGTC |
| | TGATTGTGACACTGCACTCC | AGGAGTTCGAGACGAGCCTG |
| | AGCCTGGGCAACAAAGGGA | GCCATCATGGTGAAACCCCG |
| | GACCCCTTAACTAACTAAAT | TCTCTACTAAAAATACAAAA |
| | GAATGAATGAATGAATGAAT | ATTGGCCAGGCATGGTGGCA |
| | GAAGGCAACCTGAGCTGCAT | GGCGCCTGTAATCCCAGCTG |
| | TCCTTGGCCTGCCAACCTGC | AGGCAGGAGAATCGCTTGA |
| | CCAGCCCCATCCCTCAGCCC | ACCCGGGAGGTGGAGGTTGC |
| | TCCCTGAGTCTGAGGGCCCT | AGTGAGCCGAGAGCGAGCC |
| | GCAGGTCCCACACAGGGCCA | ACTGCACTCCTGCCTGGGTA |
| | GGCTCCATCTTGTTTCTGCA | ACAAAGCAAGACTCCATCTC |
| | AATTTGCACCTTCCGTTCCAT | AAAAAAAAAAAAAAAAAAA |
| | TCCCTGCCACACTGCTCACG | AAAATCAAGTCTGGCGCAGC |
| | GGTCACCATGGATGCTGGAT | CCCAGGAAGCAATCTTGGGC |
| | AACTGCCACCGTCTCCTTGG | TGGGCACACCGCATTTCTGT |
| | AGAAGCCCTCCGTCATCTCA | GACATGAAGGCAGCGACAC |
| | GCCCCTGATGTCAGGTATTC | CTCGTCGCTGTCACAGGGTT |
| | AACCTGCAAATCCTCCCTGT | GCTGGGGTTGGGGGCTAGAG |
| | AGGTGCTGGGCTCTCCAGAG | GAGAGGAGGAGCCTCTGTTG |
| | CCCTGGGCTGTGTGCAGGGG | AGGGCTCCAAGAAGGGACA |
| | ACTCAGGGGAGAACGGGCC | GAGAGGTGGTGCTCATGGGT |
| | CCACCCAGCTCCTTCCTCAC | CCTGGAGACACCTTTTGGGT |
| | AGAGCTTTCAGAGGCTGGGG | GGTTCGTGCCCTCCATCCTG |
| | GCCTCCCTGATCCCCTCCCA | GGCCACTTTGGGGAGGTGAA |
| | GATCCCAAGGCCCCTGCTGC | GGAGGGAAGCATTAAGGGA |
| | CCCCACCCTGCAGACTGGGA | CAAGACCCCCCGTTCCCAAT |
| | CTCCGTGAGGCTGGGCTCTG | TTCTCTCCGGAGCCAGGGTT |
| | ACTTGGATATTGTGGTTCCA | TCTCTGATTCGAAGAAACAG |
| | GCACACAGCAGGCACCGTG | GTGTCACAACCCAGGAAGTC |
| | GCTGTAGTAGGCGTGCATGG | CACTGATGGCATCTGCCCTG |
| | GAAGTCAGGAGGAGCAGAC | GGGCATCAGCATTTAGGGCT |
| | CTGTCATCTCCCCTGCTGAG | GATCACTGAGGTCTGCACCT |
| | GACACAGCCCGGTCAGCGTG | CCCAAGGCTGCTGTGCCCAT |
| | TCTTGGCGGCCTGGGGCCAG | TCCTGGGCGCCCCAAAGGGG |
| | TGACTGAGCCTCTTGGCCCA | AAGAAAAACTCCTGAATGTG |
| | GTTCCTTGCCCACCTTGAAG | CACCGGGACAGGACCCATCC |
| | ATCT | CATGCGG |
| PRDX1-AKR1A1 | AGAAAATTGCCACCTATTGT | CTAGGCAGTGCTGCAAGAAC |
| | ATTCACCATATGCCAGGCAT | CAGTCTCTAAACAAACAAAG |
| | TGGGCTGGGCACTTGAGTTT | AAAAAAAAACAGGAAAAAA |
| | ATTTTTCTAATTCAAGAACA | AAAGAGTTATCCTAGCCTAG |
| | AGGCCAGGCACGGTGGCTCA | AGAAATGTTAACAGTCTATC |
| | TGCCTGTAATCCCAGCACTT | CCCTTAAGATCATAAAAGTG |
| | TGGGAGGCTAAGGCGGGCA | AAATAACTGGACAGAGTACT |
| | GATCACAAGGTCAGGAGTTT | GAAAAAAGAAGAAATCTGT |
| | GAGACCAGCCTGGCCAACAT | TGAACAAGTCTAAGTGATTT |
| | GGAGAAATCCCATCTCTACT | GTAGAGTCTCTCCAAAGGAC |
| | AAAAATACAAAAATTAGCTG | AGTAAAGATTTCAGAACCAT |
| | GGTTTCGTGGTGTGCACCTG | GGGGAGGAGGCTTTCTTGCA |
| | TAGTCCCAGCTACTCAGGGG | GGGTCTGATGTATACCTCTA |
| | GCTGAGGCAGGAGAATCTCT | GTTCTATTTTTAGATATATTC |
| | TGAACCCAGGAGGCGGAGG | CATTGTGCTCTCTTCATTTGC |
| | TTGCAGTGAGCCGAGATCGC | TCACCATTAGCATGGGAATT |
| | GCCACTGCACTCCAGCCTGA | ATTCCCCTTGTTTTATTTATA |
| | GTGACAGAGTGAGACTCCGT | TTTGTAGTAACAAGTCTAGC |
| | CTAAAAAAAAAGAACAAAA | ACAGTAACTGGCATAGAAGT |
| | GCCGGGCATGGTGGCTCAAG | ACTTAATAAATGTTACAATG |
| | CCTGTAATCCCAGCACTTTG | TGTTGAAGACTTTTGAGATG |
| | GGAGGCCAAGGTGGGCAGA | TAGAATTAAATTATTTGAAC |
| | TCATGAGGTCAGTAGTTCCA | TACGTTTCAGGAAGTTGACT |
| | GACCAGCCTGACCAACATGG | CTGGAAGCTGTGTGATGAAT |
| | TGAAAACCTGTTTCTACTAA | GAATTTAGTGACTAGGAGAT |
| | AAATACAAAAATTAGCTGGG | TAGGCAAAGAGAGCAATTG |
| | CGTGGTGGCAGGCACTTGCA | GGAGGCCACGGCAATGATCC |
| | ATCCCAGCTACTCAGGAGGC | AGACAAGAGAAGTCTCAGA |
| | TGCAGCAGGAGAATCACTTG | ACTGTAATATGACATTAGCA |
| | AACCCGGGAGGCAGAGGTTT | GCAGGGTAGAAAGAAGGAA |
| | TAGTGAGCTGAGATCCCGCC | ATACAAGGTATATTTCTTCA |
| | ACTGCACTCCAACCTGGGCA | GGCTTTAGGATTAATTGGAT |
| | ACAGAGCAAGACTCTGTCTC | TTAGGATGAAAGGAAAGAG |
| | AAAAAAAAAAAAAAAGAAA | TAAATTAAATCCAGTTTCTT |
| | AAAGAAAAAAAGAAAAGAA | GGTGACATGAGTGATGTCAT |
| | AAAAGAACTAAAAGAGAGT | TAACCTGTGACATAGGAAAC |
| | GGAGCACATTTGGCATGTGA | ACTGGAAGAAAAGCAGGTTT |
| | CTATAGTCCCAGCTACTTGG | GAGGTTTAAAATAATGTTAA |
| | TAGGCTGAGGTGAGAGGATC | ACAGGCCGGGAGCGGTGGC |
| | ACTTGAGCCCTGGAGTTTGA | TCACGCCTGTAATCCCAACA |
| | GCCCAGC | TTTT |

Confirmation that the identified intergenic region will safely support an exogenous genetic payload may be carried out by inserting a transgene at a targeted location within the intergenic region using a gene editing system. The gene editing system may be, for example, a CRISPR system (e.g., those using an CRISPR endonuclease disclosed above), a Cre/Lox system, a FLP-FRT system, a TALEN system, a ZFN system, a system that utilizes homing endonucleases, a system that produces homologous recombination, or a system that utilizes non-nuclease dependent viral vectors (e.g., retroviral, AAV, or lentiviral vectors). Constitutive, inducible, tissue-specific, or lineage-specific promoters may be used to direct expression of the inserted transgene.

In some embodiments, the targeted intergenic region is at least 100 base pairs in length. In some embodiments, the intergenic region does not comprise a promoter region or an enhancer region. While it may be better for the intergenic region not to comprise conserved regions, repetitive regions, epigenetic marks, and/or DNase hypersensitivity regions, the intergenic region may in fact contain a minimal amount of conserved regions, repetitive regions, epigenetic marks, and/or enzymatic hypersensitivity regions in some embodiments. For example, in some embodiments, the intergenic region will not comprise a CpG Island, an H3K4Me1 epigenetic mark, an H3K4Me3 epigenetic mark, an H3K27Ac epigenetic mark, a DNase I hypersensitivity region, a conserved region, or a repetitive region. However, in some embodiments, the intergenic region may comprise a CpG Island, an H3K4Me1 epigenetic mark, an H3K4Me3 epigenetic mark, an H3K27Ac epigenetic mark, a DNAseI hypersensitivity region, a conserved region, or a repetitive region. The amount of allowed conserved regions, repetitive regions, epigenetic marks, and/or DNase hypersensitivity regions depends on various factors. These factors include, for example, the size of the intergenic region; the size of the conserved, repetitive, and/or hypersensitivity regions, or epigenetic marks; the presence of gRNA binding sites; or challenges to synthesizing 5′ and 3′ homology arms for targeting and/or DNase hypersensitivity regions.

After genomic integration, the transcription level of the integrated transgene is measured and the intergenic region between the selected pair or within the selected region is confirmed to be a STAPLR when the integrated transgene displays sustained transcription (or displays sustained transcription when an inducible promoter regulating the transgene is induced).

### 3. Other Integration Sites

In some embodiments, the heterologous nucleic acid is integrated into a genomic site that is specifically active in T cells. Examples of such sites are the genes encoding a T cell receptor chain (e.g., TCR alpha chain, beta chain, gamma chain, or delta chain), a CD3 chain (e.g., CD3 zeta, epsilon, delta, or gamma chain), FOXP3, Helios, CTLA4, Ikaros, TNFR2, or CD4. In some embodiments, expression of at least a portion of the heterologous nucleic acid is driven by an endogenous gene at the site of integration. For example, in some embodiments, engineered cells comprise a heterologous nucleic acid encoding an antigen recognition receptor, such as a TCR or CAR. The nucleic acid encoding the antigen recognition receptor can be integrated into a T cell receptor locus, for example, the T cell receptor α constant (TRAC) locus. In some embodiments, the heterologous nucleic acid is integrated into Exon 1 of the TRAC locus. For example, as described in Roth et. at., Nature 2018, which is incorporated by reference.

### C. Gene-Editing Methods

Any gene editing method for the integration of a heterologous sequence into a genomic site of a target cell (e.g., an iPSC) may be used. Methods for integrating the heterologous nucleic acids into the genome can involve random integration (e.g., using lentiviruses) or can involve site-specific integration (e.g., CRISPR-based strategies). To ensure reliable, controlled expression of the heterologous nucleic acid, site-specific methods can be used to target the heterologous nucleic acid into a region in the target cell's genome that allows for consistent levels of expression of the heterologous nucleic acid integrated therein, even as the target cell undergoes changes in its differentiation state and/or regardless of cell type, e.g., the region may be a STAPLR or STEL, as referred to herein. In some embodiments, the heterologous nucleic acid can be integrated into a TCR locus allowing the expression of the heterologous nucleic acid to be regulated in a manner consistent with natural T cell development.

To enhance the precision of site-specific integration of the transgene, a construct carrying the heterologous sequence may contain on either or both of its ends a homology region that is homologous to the targeted genomic site. In some embodiments, the heterologous sequence carries both 5′ and 3′ end regions sequences that are homologous to the target genomic site in a STEL or STAPLR locus. In some embodiments, the heterologous sequence carries both 5′ and 3′ end regions sequences that are homologous to the target genomic site in a T cell specific active gene locus (e.g., a TRAC locus). The lengths of the homology regions on the heterologous sequence may be, for example, 50-1,000 base pairs in length. The homology region in the heterologous sequence can be, but need not be, identical to the targeted genomic sequence. For example, the homology region in the heterologous sequence may be at 80 or more percent (e.g., 85 or more, 90 or more, 95 or more, 99 or more percent) homologous or identical to the targeted genomic sequence (e.g., the sequence that is to be replaced by the homology region in the heterologous sequence). In further embodiments, the construct, when linearized, comprise on one end homology region 1, and on its other end homology region 2, where homology regions 1 and 2 are respectively homologous to genomic region 1 and genomic region 2 flanking the integration site in the genome.

The construct carrying the heterologous sequence can be introduced into the target cell by known techniques such as chemical methods (e.g., calcium phosphate transfection and lipofection), non-chemical methods (e.g., electroporation and cell squeezing), particle-based methods (e.g., magnetofection), and viral transduction (e.g., by using viral vectors such as vaccinia vectors, adenoviral vectors, lentiviral vectors, adeno-associated viral (AAV) vectors, retroviral vectors, and hybrid viral vectors). In some embodiments, the construct is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system. The AAV may be of any serotype, for example, AAV1, AAV2, AAV3, AAV4, AAV5, AAV6, AAV7, AAV8, AAV8.2, AAV 9, or AAVrhlO, of a pseudotype such as AAV2/8, AAV2/5, or AAV2/6.

The heterologous sequence may be integrated by site-specific gene knock-in technique. Such techniques include, without limitation, homologous recombination, gene editing techniques based on zinc finger nucleases or nickases (collectively “ZFNs” herein), transcription activator-like effector nucleases or nickases (collectively “TALENs” herein), clustered regularly interspaced short palindromic repeat systems (CRISPR, such as those using Cas9, cpf1, or PRO308), meganucleases, integrases, recombinases, and transposes. For site-specific gene editing, the editing nuclease typically generates a DNA break (e.g., a single- or double-stranded DNA break) in the targeted genomic sequence such that a donor polynucleotide having homology to the targeted genomic sequence (e.g., the construct described herein) is used as a template for repair of the DNA break, resulting in the introduction of the donor polynucleotide to the genomic site.

Gene editing techniques are well known in the art. See, e.g., U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, 9,790,490, 10,000,772, 10,113,167, and 10,113,167, each of which are incorporated by reference, for information on CRISPR gene editing techniques.

In gene editing techniques, the gene editing complex can be tailored to target specific genomic sites by altering the complex's DNA binding specificity. For example, in CRISPR technology, the guide RNA sequence can be designed to bind a specific genomic region; and in the ZFN technology, the zinc finger protein domain of the ZFN can be designed to have zinc fingers specific for a specific genomic region, such that the nuclease or nickase domains of the ZFN can cleave the genomic DNA at a site-specific manner. Depending on the desired genomic target site, the gene editing complex can be designed accordingly.

Components of the gene editing complexes may be delivered into the target cells, concurrent with or sequential to the transgene construct, by well-known methods such as electroporation, lipofection, microinjection, biolistics, virosomes, liposomes, lipid nanoparticles, immunoliposomes, poly cation or lipid nucleic acid conjugates, naked DNA or mRNA, and artificial virions. In particular embodiments, one or more components of the gene editing complex, including the nuclease or nickase, can be delivered as mRNA into the cells to be edited. In some embodiments, the gene editing complex comprises a Cas endonuclease (e.g., PRO308) and guide RNA and is delivered into the target cell in an active format, e.g., as a ribonucleoprotein (RNP).

### D. Antigen-Specificity of the Tregs

In some embodiments, the stem or progenitor cells may be further engineered (e.g., using gene editing methods described herein) to include transgenes encoding an antigen-recognition receptor such as a TCR or a CAR. Alternatively, the stem cells or progenitor cells are cells that have been reprogrammed from mature T cells, e.g., Tregs, that have already rearranged their TCR alpha/beta (or delta/gamma) loci. In some embodiments, Tregs re-differentiated from such stem or progenitor cells will retain the antigen specificity of their ancestral Tregs. Accordingly, in some embodiments, the Tregs may be selected for their specificity for an antigen of interest for a particular therapeutic goal.

In some embodiments, the antigen of interest is an autoantigen, i.e., an endogenous antigen expressed prevalently or uniquely at the site of autoimmune inflammation in a specific tissue of a subject's body. Aspects of this disclosure can provide engineered cells, e.g., Tregs, specific for such an antigen so as to home to the inflamed tissue and exert tissue-specific activity by causing local immunosuppression. Examples of autoantigens are aquaporin water channels (e.g., aquaporin-4 water channel), paraneoplastic antigen Ma2, amphiphysin, voltage-gated potassium channel, N-methyl-d-aspartate receptor (NMDAR), a-amino-3-hydroxy-5-methyl-4-isoxazoleproprionic acid receptor (AMPAR), thyroid peroxidase, thyroglobulin, anti-N-methyl-D-aspartate receptor (NR1 subunit), Rh blood group antigens, desmoglein 1 or 3 (Dsgl/3), BP 180, BP230, acetylcholine nicotinic postsynaptic receptors, thyrotropin receptors, platelet integrin, glycoprotein IIb/IIIa, calpastatin, citrullinated proteins, alpha-beta-crystallin, intrinsic factor of gastric parietal cells, phospholipase A2 receptor 1 (PLA2R1), and thrombospondin type 1 domain-containing 7A (THSD7A). Additional examples of autoantigens are multiple sclerosis-associated antigens (e.g., myelin basic protein (MBP), myelin associated glycoprotein (MAG), myelin oligodendrocyte glycoprotein (MOG), proteolipid protein (PLP), oligodendrocyte myelin oligoprotein (OMGP), myelin associated oligodendrocyte basic protein (MOBP), oligodendrocyte specific protein (OSP/Claudin 11), oligodendrocyte specific proteins (OSP), myelin-associated neurite outgrowth inhibitor NOGO A, glycoprotein Po, peripheral myelin protein 22 (PMP22), 2′3′-cyclic nucleotide 3′-phosphodiesterase (CNPase), and fragments thereol); joint-associated antigens (e.g., citrulline-substituted cyclic and linear filaggrin peptides, type II collagen peptides, human cartilage glycoprotein 39 peptides, keratin, vimentin, fibrinogen, and type I, III, IV, and V collagen peptides); and eye-associated antigens (e.g., retinal arrestin, S-arrestin, interphotoreceptor retinoid-binding proteins, beta-crystallin B1, retinal proteins, choroid proteins, and fragments thereof). In some embodiments, the autoantigen is relevant for the treatment of Crohn's disease, inflammatory bowel disease, or rheumatoid arthritis. In some embodiments the autoantigen is relevant for the treatment of MS, for example MOG or MBP. In some embodiments, the autoantigen is relevant to the treatment of type-1-diabetes (for example, insulin). In some embodiments, the Tregs may target other antigens of interest (e.g., B cell markers CD19 and CD20). In some embodiments, Tregs may be administered to patients for whom standard care has not worked. For example, a patient group of IBD patients that are TNFa blocker therapy refractory.

In some embodiments, the antigen of interest is a polymorphic allogeneic MHC molecule, such as one expressed by cells in a solid organ transplant or by cells in a cell-based therapy (e.g., bone marrow transplant, cancer CAR T therapy, or cell-based regenerative therapy). MHC molecules, without limitation, HLA-A, HLA-B, or HLA-C; HLA-DP, HLA-DM, HLA-DOA, HLA-DOB, HLA-DQ, or HLA-DR. By way of example, the antigencan bea class I molecule HLA-A2. HLA-A2 is a commonly mismatched histocompatibilityhaplotype in transplantation. Engineered Tregs expressing a CAR specific for an MHC class I antigen are advantageous because MHC class I molecules are broadly expressed on all tissues, so the Tregs can be used for organ transplantation regardless of the tissue type of the transplant. Tregs against HLA-A2 antigen can provide an additional advantage because HLA-A2 is expressed by a substantial proportion of the human population and therefore on many donor organs. There has been evidence showing that expression of an HLA-A2 CAR in Treg cells can enhance the potency of the Treg cells in preventing transplant rejection.

In some embodiments, the engineered cell can be selected to recognize foreign peptides (e.g., CMV, EBV, and HSV), rather than allo-antigens, and can be used in an allogeneic adoptive cell transfer setting without the risk of being constantly activated by recognizing allo-antigens and without the need for knockout of TCR expression.

In some embodiments, the engineered cells are derived from T cells of a subject in which the T cells comprise a TCR capable of recognizing a peptide of interest. For example, the T cells can be derived from a subject inflicted with an autoimmune disease. In some embodiments, the T cells expressing a TCR of interest (e.g., a TCR that recognizes MBP) are isolated from the subject-derived T cells. The isolated T cells can be reprogrammed into iPSCs as described herein. Advantageously, upon differentiation of the T cell derived iPSCs, the developing T cells will express the TCR of interest.

### E. Cells Used for Genome Editing

The engineered cells of the present disclosure include mammalian cells, such as human cells, cells from a farm animal (e.g., a cow, a pig, or a horse), and cells from a pet (e.g., a cat or a dog). The source cells, i.e., cells on which genome editing is performed, may be pluripotent stem cells (PSCs). PSCs are cells capable to giving rise to any cell type in the body and include, for example, embryonic stem cells (ESCs), PSCs derived by somatic cell nuclear transfer, and induced PSCs (iPSCs).

In some embodiments, the source cells for genome editing are multipotent cells such as hematopoietic stem cells (e.g., those isolated from bone marrow or cord blood), or hematopoietic progenitor cells (e.g., lymphoid progenitor cells). Multipotent cells are capable of differentiating into more than one cell type but are more limited in cell type potential than pluripotent cells. The multipotent cells may be derived from established cell lines or isolated from human bone marrow or umbilical cords. By way of example, the hematopoietic stem cells (HSC) may be isolated from a subject or a healthy donor following granulocyte-colony stimulating factor (G-CSF)-induced mobilization, plerixafor-induced mobilization, or a combination thereof. To isolate HSCs from the blood or bone marrow, the cells in the blood or bone marrow may be panned by antibodies that bind unwanted cells, such as antibodies to CD4 and CD8 (T cells), CD45, GR-1 (granulocytes). HSCs can then be positively selected by antibodies to CD34. In some embodiments, the cells to be engineered are iPSCs reprogrammed from a mature Treg, such as a mature Treg expressing a TCR that targets a non-allogenic antigen. In some embodiments, the edited stem cells and/or progenitor cells may be differentiated into a CD4+ T cell. In some embodiments, the CD4+ T cell can be further differentiated into a T cell subtype, e.g., a Treg cell, in vitro before engrafting into a subject, as further discussed below. Alternatively, the stem and/or edited progenitor cells may be induced to differentiate into Treg cells after engrafting to a subject.

In some embodiments, the engineered cells are reprogrammed somatic cells, e.g., iPS cells. For example, in some embodiments, it may be desirable to use reprogrammed T cells (e.g., CD4+ T cells or derivatives thereof) as the reprogrammed cell may retain epigenetic features that improve its efficacy once the engineered cells have been re-differentiated back into T cells, e.g., a CD4+ T cells or derivatives thereof. For example, a stem cell reprogrammed from a T cell may retain features of an arranged TCR locus which may enhance differentiation potential of the stem cell back into a T cell.

In some embodiments, the engineered cells are reprogrammed from T cells. The T cells to be used for reprogramming may be isolated from a number of sources, including peripheral blood mononuclear cells (PBMC), bone marrow, lymph node tissue, cord blood, thymus tissue, or spleen tissue. For example, T cells may be isolated from a unit of blood collected from a subject using well known techniques such as, for example, Ficoll separation, centrifugation through a PERCOLL gradient following red blood cell lysis and monocyte depletion, counterflow centrifugal elutriation, leukapheresis, and subsequent cell surface marker-based magnetic or flow cytometric isolation.

In some embodiments, the T cells for reprogramming are enriched from isolated cells. For example, T cells can be isolated by positive and/or negative selection with a combination of antibodies directed to unique surface markers using techniques such as flow cytometry cell sorting and/or magnetic immunoadherence involving conjugated beads. For example, to enrich for CD4+ cells by negative selection, a monoclonal antibody cocktail typically may include antibodies to CD14, CD20, CD11b, CD16, HLA-DR, and CD8. To enrich or positively select for CD4+ T cells, antibodies to CD4, CD3, and CD45 can be used.

### F. Additional Genome Editing

The engineered cells may be further genetically engineered, before or after the genome editing described above, to make the cells more effective, more useable on a larger subject population, and/or safer. The genetic engineering may be done by, e.g., random insertion of a heterologous sequence of interest (e.g., by using a lentiviral vector, a retroviral vector, or a transposon) or targeted genomic integration (e.g., by using genome editing mediated by ZFN, TALEN, CRISPR, site-specific engineered recombinase, or mega nuclease).

For example, the cells may be engineered to express one or more exogenous CAR or TCR through a site-specific integration of a CAR or TCR transgene into the genome of the cell. The exogenous CAR or TCR may target an antigen of interest, as described above.

The cells may also be edited to encode one or more therapeutic agents to promote an immunosuppressive activity. Examples of therapeutic agents include cytokines (e.g., IL-10), chemokines or their receptors (e.g., CCR7), growth factors (e.g., remyelination factors for treatment of multiple sclerosis), and signaling factors (e.g., amphiregulin).

In additional embodiments, the cells can be further engineered to express a factor that reduces severe side effects and/or toxicities of cell therapy, such as cytokine release syndrome (CRS) and/or neurotoxicity.

In some embodiments, EZH1 signaling is disrupted in the engineered cells to enhance their lymphoid commitment (see, e.g., Vo et al, Nature (2018) 553(7689):506-10).

In some embodiments, the engineered cells may be allogeneic cells to the subject in need of treatment. In such instances, the cells may be further engineered to reduce host rejection to these cells (graft rejection) and/or these cells' potential attack on the host (graft-versus-host disease). The further-engineered allogeneic cells are particularly useful because they can be used in multiple subjects without compatibility issues. The allogeneic cells thus can be called “universal” and can be used “off the shelf” The use of “universal” cells greatly improves the efficiency and reduces the costs of adopted cell therapy.

In some embodiments, it may be desirable for the engineered cells to contain a “safety switch” in their genomes, such that proliferation or activity of the cells can be stopped when their presence in the subject is no longer desired. A safety switch may, for example, be a suicide gene, which upon administration of a compound to the subject, will be activated or inactivated such that the cells enter apoptosis. A suicide gene may encode an enzyme not found in humans (e.g., a bacterial or viral enzyme) that converts a harmless substance into a toxic metabolite in the human cell.

### G. Reprogramming and Differentiating Cells In Vitro

The cells useful in the compositions and methods of the present disclosure can be reprogrammed from mature cells and/or differentiated into T cells (e.g., CD4+ T cells or derivatives thereof, e.g., Treg cells) in tissue culture. The methods described below are merely illustrative and are not limiting.

1. Reprogramming Cells into iPSCs

In some embodiments, this disclosure provides iPSCs derived from differentiated cells, e.g., T cells. The differentiated cells, such as T cells, can be reprogrammed into iPSCs using reprogramming factors such as OCT3/4, SOX2, KLF4, and c-MYC (or L-MYC) (see, e.g., Nishino et al., Regen Ther (2018) 9:71-8; U.S. Pat. No. 8,048,999 B2 each of which are incorporated by reference). Reprogramming factors may be delivered via non-integrating methods (e.g., Sendai virus, plasmid, RNA, mini circle, AAV, IDLV, etc.) or integrating methods (e.g., lentivirus, retrovirus, and nuclease-mediated targeted integration).

2. Differentiation of Stem Cells into CD34+ Cells

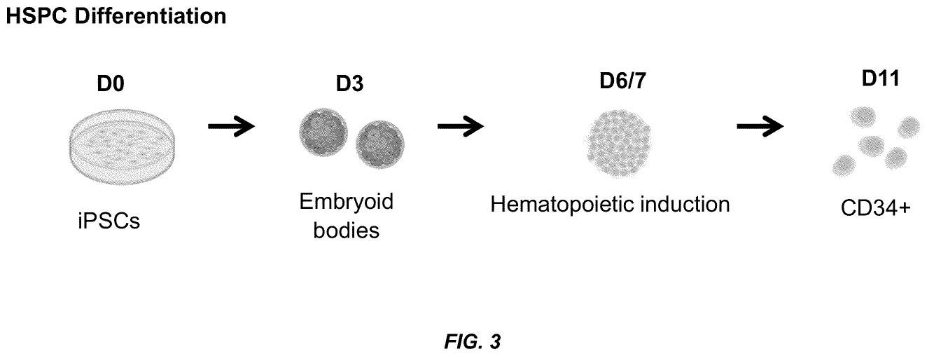

FIG. 3 illustrates an exemplary cell culture procedure for generating CD34+ cells (HSPCs) from stem cells. On Day 0 (DO) stem cells (iPSCs) are plated into tissue culture dishes in a base stem cell media (i.e., StemPro34 with supplement, ITSG, Glut, Vitamin C, Non-Essential Amino Acids (NEAA), BME, P/S) combined with the following factors: FGF2 (1-100 ng/ml); BMP4 (1-200 ng/ml); ROCKi (1-20 uM); CHIR99021 (1-20 uM). At Day 3 (D3) the cultured cells are developing as embryoid bodies, and the media is changed to base stem cell media comprising the following factors: FGF2 (1-100 ng/ml); VEGF (1-100 ng/ml); SCF (1-100 ng/ml); FLT3L (1-100 ng/ml); IL-3 (1-100 ng/ml). At approximately Days 6-7 (D6/7), hematopoietic induction occurs. Media are changed to base stem cell media comprising the following factors: TPO (1-100 ng/ml); VEGF (1-100 ng/ml); SCF (1-100 ng/ml); FLT3L (1-100 ng/ml); IL-3 (1-100 ng/ml); FGF2 (1-100 ng/ml). At approximately Day 11 (D11) CD34+ cells (HSPCs) can be isolated from tissue culture dishes. In some embodiments, it may be desirable to enrich for CD34+ cells. To enrich for CD34+ cells, the cells are subjected to CD34+ magnetic bead purification or FACS sorting.

FIG. 4 illustrates an exemplary cell culture procedure for generating CD4+ T cells from CD34+ cells. On Day 0 (DO) (counting DO as the plating of CD34+ positive cells), CD34+ cells are plated into tissue culture dishes with OP9-DLL4 cells or into dishes coated with DLL4/Retronectin coating in a differentiation media (e.g., aMEM with 20% FBS, ITSG, Glut, Vitamin C, BME, P/S) comprising the following factors: IL-7 (1-100 ng/ml); SCF (1-100 ng/ml); TPO (1-100 ng/ml); FLT3L (1-100 ng/ml); IL-2 0-100 (U/ml). At Day 5 (D5) the media is changed to differentiation media comprising the following factors: IL-7 (1-100 ng/ml); SCF (1-100 ng/ml); TPO (1-100 ng/ml); FLT3L (1-100 ng/ml); IL-2 0-100 (U/ml). At Day 10 (D10) the media is changed to differentiation media comprising the following factors: IL-7 (1-100 ng/ml); SCF (1-100 ng/ml); TPO (1-100 ng/ml); FLT3L (1-100 ng/ml); IL-2 0-100 (U/ml). At Day 15 (D15) the media is changed to differentiation media comprising the following factors: IL-7 (1-100 ng/ml); SCF (1-100 ng/ml); TPO (1-100 ng/ml); FLT3L (1-100 ng/ml); IL-2 0-100 (U/ml). At Day 20 (D20) the media is changed to differentiation media comprising the following factors: IL-7 (1-100 ng/ml); SCF (1-100 ng/ml); TPO (1-100 ng/ml); FLT3L (1-100 ng/ml); IL-2 0-100 (U/ml). At Day 25 (D25) the media is changed to differentiation media comprising the following factors: IL-7 (1-100 ng/ml); SCF (1-100 ng/ml); TPO (1-100 ng/ml); FLT3L (1-100 ng/ml); IL-2 0-100 (U/ml). After Day 25 the cells can be further stimulated towards CD4+ T cells. Half-medium changes are performed 2 or 3 days after passaging the cells onto a new feeder layer or freshly DLL4/retronectin coated well.

### 3. Differentiation of CD4+ T Cells Towards CD4+ T Cell Subtypes

CD4+ T cells can be differentiated in vitro into other CD4+ T cell subtypes, such as Tregs. Tregs can be generated from CD4+ T cells through a combination of molecules, such as IL-2, rapamycin, retinoic acid, TGF-beta, and/or butyrate, upon stimulation with anti-human CD3 and CD28 antibodies. Th1 cells can be generated from CD4+ T cells through a combination of molecules, such as IL-2 and IL-12, upon stimulation with anti-human CD3 and CD28 antibodies. Th2 cells can be generated from CD4+ T cells through a combination of molecules, such as IL-2 and IL-4, upon stimulation with anti-human CD3 and CD28 antibodies. Th17 cells can be generated from CD4+ T cells through a combination of molecules, such as IL1beta, IL-6, IL-23 or TGF-beta+IL-6, upon stimulation with anti-human CD3 and CD28 antibodies.

Plasticity is the phenomenon that a given cell type can differentiate into a slightly different subtype. This phenomenon is well documented in T helper cell biology and is often driven by the immunological milieu, proximity of other immune cells and inflammatory mediators. It appears that Treg cells are able to lose FOXP3 expression and acquire an effector phenotype (a state known as exTreg) under inflammatory and environmental conditions. To maintain the Treg phenotype and/or to increase expression of the transgene(s) (e.g., FOXP3, Helios, and/or ThPOK) in the engineered Treg cells, the cells may be cultured in tissue culture media containing rapamycin and high-dose IL-2.

### H. Use of CD4+ T Cells and Derivatives Thereof

The CD4+ T cells, and derivatives thereof (e.g., Tregs), generated according to aspects of the present disclosure can be used in cell therapy to treat a subject (e.g., a human subject) in need of induction of immune tolerance or restoration of immune homeostasis. A subject herein may be having or at risk of having an undesired inflammatory condition such as an autoimmune disease. Examples of autoimmune diseases are Addison's disease, ankylosing spondylitis, anti-glomerular basement membrane disease autoimmune hepatitis, dermatitis, Goodpasture's syndrome, granulomatosis with polyangiitis, Graves' disease, Guillain-Barre syndrome, Hashimoto's thyroiditis, hemolytic anemia, Henoch-Schonlein purpura (HSP), juvenile arthritis, juvenile myositis, Kawasaki disease, inflammatory bowel diseases (i.e. Crohn's disease and ulcerative colitis), polymyositis, pulmonary alveolar proteinosis, multiple sclerosis, myasthenia gravis, neuromyelitis optica, PANDAS, psoriasis, psoriatic arthritis, rheumatoid arthritis, Sjogren's syndrome, systemic scleroderma, systemic sclerosis, systemic lupus erythematosus, thrombocytopenic purpura (TTP), Type I diabetes mellitus, uveitis, vasculitis, vitiligo, and Vogt-Koyanagi-Harada Disease.

In some embodiments, stem or progenitor cells of the present disclosure are engineered to express an antigen-binding receptor (e.g., TCR or CAR) targeting an autoantigen associated with an autoimmune disease, such as, insulin, myelin oligodendrocyte glycoprotein (multiple sclerosis), myelin protein zero (autoimmune peripheral neuropathy), myelin basic protein (multiple sclerosis), CD37 (systemic lupus erythematosus), CD20 (B-cell mediated autoimmune diseases).

A subject herein may be one in need of an allogeneic transplant, such as an allogeneic tissue or solid organ transplant or an allogeneic cell therapy. The CD4+ T cells (including CD4+ Tregs) of the present disclosure, such as those expressing TCRs targeting one or more allogeneic MHC class I or II molecules, may be introduced to the subject, where the CD4+ T cells will home to the transplant and suppress allograft rejection elicited by the host immune system and/or graft-versus-host rejection. Subjects in need of a tissue or organ transplant or an allogeneic cell therapy include those in need of, for example, kidney transplant, heart transplant, liver transplant, pancreas transplant, intestine transplant, vein transplant, bone marrow transplant, and skin graft; those in need of regenerative cell therapy; those in need of gene therapy (AAV-based gene therapy); and those in need of cancer CAR T therapy.

If desired, the subject receiving the engineered cells herein is treated with a mild myeloablative procedure prior to introduction of the cell graft or with a vigorous myeloablative conditioning regimen.

The CD4+ T cells of the present disclosure may be provided in a pharmaceutical composition containing the cells and a pharmaceutically acceptable carrier. For example, the pharmaceutical composition comprises sterilized water, physiological saline or neutral buffered saline (e.g., phosphate-buffered saline), salts, antibiotics, isotonic agents, and other excipients (e.g., glucose, mannose, sucrose, dextrans, mannitol; proteins (e.g., human serum albumin); amino acids (e.g., glycine and arginine); antioxidants (e.g., glutathione); chelating agents (e.g., EDTA); and preservatives). The pharmaceutical composition may additionally comprise factors that are supportive of a T cell (e.g., Treg) phenotype and growth (e.g., IL-2 and rapamycin or derivatives thereof), anti-inflammatory cytokines (e.g., IL-10, TGF-β, and IL-35), and other cells for cell therapy (e.g., CAR T effector cells for cancer therapy or cells for regenerative therapy). For storage and transportation, the cells optionally may be cryopreserved. Prior to use, the cells may be thawed and diluted in a pharmaceutically acceptable carrier.

A pharmaceutical composition according to aspects of this disclosure can be administered to a subject in a therapeutically effective amount through systemic administration (e.g., through intravenous injection or infusion) or local injection or infusion to the tissue of interest (e.g., infusion through the hepatic artery, and injection to the CNS (brain, spinal cord), heart, or muscle).

Accordingly, in some aspects, this disclosure provides a method for treating a subject diagnosed with a cancer or a subject in need of immunosuppression by administering an engineered cell as described herein. In some aspects, this disclosure provides a method for treating a subject diagnosed with a cancer or a subject in need of immunosuppression by administering a cell derived from the stem or progenitor T cell contained in any of the compositions provided herein. In some aspects, this disclosure provides a method for treating a subject diagnosed with a cancer or a subject in need of immunosuppression by administering a CD4+ T cell or a population of cells enriched for CD4+ T cells obtained from any of the methods provided herein.

In some embodiments, the subject is a recipient of a cell or tissue transplant. In some embodiments, the subject has an autoimmune disease. In some aspects, this disclosure provides a use of an engineered cell, as disclosed herein, in the manufacture of a medicament in treating a cancer or a subject in need of immunosuppression.

In some aspects, this disclosure provides a method for treating a subject diagnosed with a cancer by administering an engineered cell as described herein. Accordingly, in some embodiments, this disclosure makes use of CD4+ T cells as anti-tumor effector cells.

In some embodiments, a single dosing unit of the pharmaceutical composition comprises more than 10\hat{ }4 cells (e.g., from about 10\hat{ }5 to about 10\hat{ }6 cells, from about 10\hat{ }6 to about 10\hat{ }10, from about 10\hat{ }6 to 10\hat{ }7, from about 10\hat{ }6 to 10\hat{ }8, from about 10\hat{ }7 to 10\hat{ }8, from about 10\hat{ }7 to 10\hat{ }9, or from about 10\hat{ }8 to 10\hat{ }9 cells). In certain embodiments, a single dosing unit of the composition comprises about 10\hat{ }6, about 10\hat{ }7, about 10\hat{ }8, about 10\hat{ }9, or about 10\hat{ }10 or more cells. The subject may be administered with the pharmaceutical composition once every two days, once every three days, once every four days, once a week, once every two weeks, once every three weeks, once a month, or at another frequency as necessary to establish a sufficient population of engineered Treg cells in the subject.

### II. ARTIFICIAL ANTIGEN PRESENTING SUBSTRATES

In vitro systems for generating CD4+ T cells from stem or progenitor cells (e.g., progenitor T cells) remain lacking at least in part due to the spatiotemporal complexity of T cell development. For example, during T cell development progenitor cells undergo multiple maturation stages within a unique microenvironment of the thymus where they experience significant changes in transcriptional networks and respond to a multitude of signaling factors to generate a diverse array of T cells.

The different maturation stages are often characterized based on the presence or absence of specific cell surface markers. For example, the earliest developmental stage is often called the “double negative” or “DN” stage in reference to an absence of expression for both CD4 and CD8 surface markers. The DN cells can be further divided into at least four stages (DN1-DN4) according to the expression of CD44 and CD25: DN1 (CD44+CD25−), DN2 (CD44+CD25+), DN3 (CD44−CD25+), and DN4 (CD44−CD25−). DN1 cells generally proliferate extensively under the stimuli provided by the thymus and progress to become DN2 population. The DN2 cells often continue to express considerable numbers of genes related to stemness, and also express of IL-2 receptor (CD25) and IL-7 receptor (CD127), which is associated with an acquired T cell identity. Expression of RAG1/2 recombinases and T cell receptor (TCR) signaling complex proteins such as CD3 epsilon and Zap70 may occur through the transition to DN3 stage. At the DN3 stage, rearrangement and expression of functional TCR beta chains can occur through association with pre-T alpha chains, which can provide pre-TCR signals necessary for DN3 thymocytes to advance to DN4 stage. DN4 cells finally recombine and express functional TCR alpha chain and competency of the TCR alpha beta chain expression will further drive DN4 cells to develop into double-positive (DP) cells, which is in reference to their positive expression of both CD4 and CD8.

In some aspects, this disclosure overcomes challenges posed by a complex spatiotemporal developmental pathway by identifying and incorporating key biological components of T cell development into a cell culture-based platform that is capable of producing CD4+ T cells in vitro. For example, one insight of this disclosure is the recognition that certain interactions between HLA molecules and their restricted TCRs, in combination with appropriate signals via co-stimulatory receptors, can enhance formation of CD4+ T cells from stem or progenitor cells. Accordingly, this disclosure provides compositions and methods for modulating interactions (e.g., strength and duration of interaction) between HLA molecules (e.g., HLA class II molecules) and TCRs, as well as providing appropriate co-stimulatory signals, to generate CD4+ T cells (or CD4+ T cell derivatives) for therapeutic use, which can include cell therapy in autoimmunity, cancer, and immune system homeostasis.

In particular, certain aspects of this disclosure provide compositions and methods that make use of artificial antigen presenting substrates (aAPSs) (e.g., artificial antigen presenting cells (aAPCs)) to control interactions between HLA molecules and the antigen recognition receptors (e.g., TCRs) of emerging progenitor T cells (e.g., DN4 or DP cells) for efficient generation of CD4+ T cells in vitro. In some embodiments, artificial antigen presenting substrates are used to interact with antigen recognition receptors (e.g., TCRs) of progenitor T cells, via HLA molecules (e.g., HLA-class II molecule) on the surface of the artificial antigen presenting substrates, such that a functional signal of sufficient duration and strength is generated to facilitate the differentiation of the progenitor T cells (e.g., DN4 or DP cells) into CD4+ T cells. The duration of the interaction can be tightly controlled through co-culture or removal of the artificial antigen presenting cells. The signal strength can also be controlled through the ratio of progenitor T cells to artificial antigen presenting cells. The signal strength can be controlled through the extent of surface expression of the HLA molecules of antigen presenting substrates and/or surface expression of antigen recognition receptors of progenitor T cells. The signal strength can also be controlled through selection of co-stimulatory molecules on the artificial antigen presenting substrates.

In some embodiments, sufficient duration and strength of signal is generated on account of the HLA molecule of the artificial antigen presenting substrate providing a cognate antigen to the antigen recognition receptor of, for example, the stem cell derived progenitor T cell. The cognate antigen can also be leveraged to determine signal strength between antigen recognition receptor and HLA molecule (e.g., HLA-class II molecule). In particular, small amino acid changes in the cognate antigen can be used to enhance T cell activation through the antigen recognition receptor. Furthermore, to enhance interactions between the HLA molecule and the antigen recognition receptor, in some embodiments, the cognate antigen is linked to the HLA molecule, e.g., HLA class II molecule, of the artificial antigen presenting substrate via a peptide linker.

In some embodiments, the strength of interactions between HLA molecules of the artificial antigen presenting substrate and TCRs of stem or progenitor cells are modulated based on the selection of one or more co-stimulatory molecules incorporated on the artificial antigen providing substrates. In some embodiments, the HLA molecule is an HLA class II molecule, for example, one of HLA-DP, HLA-DM, HLA-DO, HLA-DQ HLA-DR, and the one or more of co-stimulatory comprise one or more of CD40, CD80, CD83, CD86, ICOS-L, CD58, and ICAM1 (sometimes referred to as CD54). The HLA class II molecule can be selected such that it matches its respective antigen recognition receptor (TCR), i.e., HLA-class II molecule is restricted to the TCR. In some embodiments, the HLA class II molecule is HLA-DR and the one or more co-stimulatory molecule is CD80. In some embodiments, the HLA class II molecule is HLA-DR and the one or more co-stimulatory molecule is CD80 and ICAMI.

In some embodiments, the artificial antigen presenting substrate is a cell (e.g., aAPC). For example, the antigen presenting substrate can be an adherent cell, such as OP9 cells, dendritic cells. In other embodiments, the antigen presenting substrate can be a cell that is cultured in suspension. In some embodiments, an exogenous nucleic acid encoding the HLA molecule that, upon expression can present an antigen to a progenitor T cell, is introduced into the cell. The nucleic acid encoding the HLA molecule can be integrated into the cell by any means known in the art, including transduction, such as lentiviral transduction, or with gene-editing systems such as CRISPRs. Accordingly, in some embodiments, the exogenous nucleic acid encoding the HLA molecule can be integrated into the genome of the cell. In some embodiments, the exogenous nucleic acid encoding the HLA molecule further comprises a sequence encoding a peptide antigen to be presented to the antigen recognition receptor of a progenitor T cell. The HLA molecule can be linked with the peptide antigen via a linker sequence. Advantageously, linking the peptide antigen to the HLA molecule enhances the efficiency of interactions between the antigen recognition receptor and cognate antigen.

In some embodiments, the artificial antigen presenting substrate comprises a bead. For example, in some embodiments, the artificial antigen presenting substrate comprises an agarose, Sepharose, polystyrene, or polyethylene glycol bead. In some embodiments, the artificial antigen presenting substrate comprises a particle, such as, for example, a nanoparticle. In some embodiments, the nanoparticle comprises a polymer, such as, biodegradable PLGA.

In some embodiments, the duration of interactions between HLA molecules of the artificial antigen presenting substrate and antigen recognition receptors (e.g., TCRs) of progenitor T cells are modulated based on the amount of time in which the progenitor T cells are contacted with the artificial antigen presenting substrates. In some embodiments, the progenitor T cells are contacted with the artificial antigen presenting substrate for less than 24 hours. In some embodiments, the progenitor T cells are contacted with the artificial antigen presenting substrate for at least 24 hours, for example, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days.

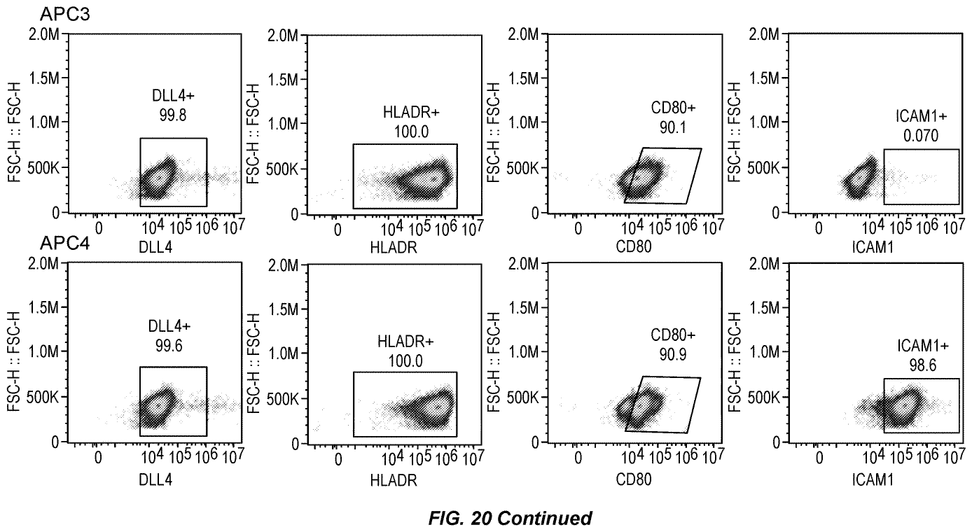

In some embodiments, the stem or progenitor T cells are cultured with at least two different aAPSs. For example, in some embodiments, a progenitor T cell is cultured with a first aAPS during a first stage of T cell differentiation and is cultured with a second aAPS during a second stage of T cell differentiation. In some embodiments, the first and second aAPS are different. For example, making reference to FIG. 20, the first aAPS may be one of APC1-APC3 and the second aAPS may be APC4.

In some embodiments, compositions and methods of the disclosure make use of aAPSs to generate CD4+ T cells, and derivatives thereof, from progenitor T cells by providing to the progenitor T cells a cognate antigen-presenting HLA molecule recognizable by the TCR of the progenitor T cell. In some embodiments, the stage of T cell development during which the cognate antigen-presenting HLA molecule interacts with the TCR of the developing progenitor T cells is an important factor for generating CD4+ T cells. In some embodiments, the progenitor T cells are contacted with the artificial antigen presenting substrate when the progenitor T cells has a CD34+ phenotype. In some embodiments, the progenitor T cells are contacted with the artificial antigen presenting substrate when the progenitor T cells has a DN phenotype. For example, the progenitor T cells can be contacted with the artificial antigen presenting substrate when the progenitor T cells has a DN1 (CD44+CD25−) phenotype, a DN2 (CD44+CD25+) phenotype, a DN3 (CD44−CD25+) phenotype, or a DN4 (CD44−CD25−) phenotype. In some embodiments, the progenitor T cells are contacted with the artificial antigen presenting substrate when the progenitor T cells comprise a CD4+CD8+ double positive DP phenotype.

In some embodiments, the artificial antigen presenting substrate is modified to express a Notch ligand. For example, in some embodiments, an exogenous nucleic acid encoding a Notch ligand is introduced into the artificial antigen presenting substrate. The exogenous nucleic acid encoding the Notch ligand can be introduced into the artificial antigen presenting substrate via any means known in the art including, for example, lentiviral transduction. The Notch ligand can be a DLL1 or a DLL4 Notch ligand.

In some embodiments, the artificial antigen presenting substrate is modified to secrete one or more cytokines or growth factors, e.g., TGF-β.

In some embodiments, the artificial antigen presenting substrate is a cell. For example, an OP9 cell. In some embodiments, the artificial antigen presenting substrate is engineered to express a Notch ligand. For example, in some embodiments the artificial antigen presenting substrate is engineered to express human DLL4. In some embodiments, the artificial antigen presenting substrate is modified so as to express an HLA class II molecule, e.g., HLA-DR. In some embodiments, the artificial antigen presenting substrate is further modified to express a co-stimulatory ligand, e.g., CD80 and/or ICAM9. In some embodiments, the artificial antigen presenting substrate expresses a Notch ligand (human DLL4), HLA-DR, and CD80. In some embodiments, the substrate expresses a Notch ligand (human DLL4), HLA-DR, CD80, and ICAMI.

Accordingly, in some aspects this disclosure provides a system for the efficient generation of CD4+ T cells and CD4+ T cell subtypes by providing methods and compositions for modulating interactions between immunomodulatory polypeptides or antigen recognition receptors of progenitor T cells and artificial antigen presenting substrates. Advantageously, the system described herein can be a 2D system.

In order that this invention may be better understood, the following examples are set forth. These examples are for purposes of illustration only and are not to be construed as limiting the scope of the invention in any manner.

### III. ILLUSTRATIVE EMBODIMENTS

The following paragraphs are not claims, but represent illustrative embodiments of the disclosure:

Embodiment 1: An engineered progenitor T cell comprising:
a heterologous nucleic acid that, when activated, results in the expression of a lineage commitment factor at a level that is equivalent or higher than that of a comparable wild-type cell to thereby facilitate differentiation towards a CD4+ T cell.

Embodiment 2: The engineered progenitor T cell of embodiment 1, wherein the engineered progenitor T cell comprises a CD4+CD8+ double positive (DP) phenotype.

Embodiment 3: The engineered progenitor T cell of embodiment 1, wherein the engineered progenitor T cell comprises a CD4−CD8− double negative (DN) phenotype.

Embodiment 4: The engineered progenitor T cell of any one of embodiments 1 to 3, wherein the heterologous nucleic acid is expressed by the engineered progenitor T cell.

Embodiment 5: The engineered progenitor T cell of any one of embodiment 1 to 4, wherein the lineage commitment factor comprises at least one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

Embodiment 6: The engineered progenitor T cell of any one of embodiments 1 to 5, wherein the heterologous nucleic acid encodes the lineage commitment factor.

Embodiment 7: The engineered progenitor T cell of any one of embodiments 1 to 6, wherein the lineage commitment factor comprises ThPOK.

Embodiment 8: The engineered progenitor T cell of any one of embodiments 1 to 7, wherein the heterologous nucleic acid is integrated in the genome of the engineered progenitor T cell in a sustained transcriptionally active payload region (STAPLR) or a sustained transgene expression locus (STEL).

Embodiment 9: The engineered progenitor T cell of embodiment 8, wherein the STAPLR is selected from the group consisting of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene; and
the intergenic region between the PA2G4 gene and the RPL41 gene.

Embodiment 10: The engineered progenitor T cell of embodiment 9, wherein the STAPLR is the intergenic region between the PRDX1 gene and the AKR1A1 gene.

Embodiment 11: The engineered progenitor T cell of any one of embodiments 1 to 10, wherein the heterologous nucleic acid is integrated at a location that is at least 100-5000 base pairs away from the nearest gene.

Embodiment 12: The engineered progenitor T cell of any one of embodiments 1 to 7, wherein the heterologous nucleic acid is integrated into the genome of the engineered progenitor T cell at or near a gene that is specifically expressed in a T cell.

Embodiment 13: The engineered progenitor T cell of any one of embodiments 1 to 7, wherein the heterologous nucleic acid is integrated in the genome of the engineered progenitor T cell at or near a TRAC locus.

Embodiment 14: The engineered progenitor T cell of any one of embodiments 1 to 13, wherein the heterologous nucleic acid further comprises an inducible promoter upstream of the lineage commitment factor.

Embodiment 15: The engineered progenitor T cell of any one of embodiments 1 to 14, wherein the heterologous nucleic acid, when activated, results in at least a 2-fold increase in expression of the lineage commitment factor as compared to the comparable wild-type cell under similar conditions and thereby steers differentiation of the engineered cell into the CD4+ T cell.

Embodiment 16: The engineered progenitor T cell of any one of embodiments 1 to 15, wherein the engineered progenitor T cell further comprises a heterologous nucleic acid sequence encoding a second and optionally a third lineage commitment factor.

Embodiment 17: The engineered progenitor T cell of embodiment 16, wherein the second lineage commitment factor and optionally the third lineage commitment factor, when activated, promotes differentiation towards a CD4+ T cell or a CD4+ T cell subtype.

Embodiment 18: The engineered progenitor T cell of embodiment 17, wherein the T cell subtype comprises a Treg cell.

Embodiment 19: The engineered progenitor T cell of any one of embodiments 1 to 18, wherein the engineered progenitor T cell is derived from an induced pluripotent stem cell.

Embodiment 20: The engineered progenitor T cell of embodiment 19, wherein the induced pluripotent stem cell is derived from a T cell.

Embodiment 21: The engineered progenitor T cell of embodiment 20, wherein the T cell comprises an autoantigen specific TCR.

Embodiment 22: The engineered progenitor T cell of any one of embodiments 1 to 21, wherein the engineered progenitor T cell is genetically modified to integrate an exogenous nucleic acid encoding a T cell receptor (TCR) or a chimeric antigen receptor (CAR).

Embodiment 23: The engineered progenitor T cell of embodiment 22, wherein the exogenous nucleic acid encodes the TCR and the TCR is specific to an autoantigen.

Embodiment 24: The engineered progenitor T cell of embodiment 23, wherein the exogenous nucleic acid is integrated within a STEL.

Embodiment 25: The engineered progenitor T cell of embodiment 24, wherein the STEL comprises a housekeeping gene.

Embodiment 26: The engineered progenitor T cell of embodiment 25, wherein the housekeeping gene comprises GAPDH.

Embodiment 27: The engineered progenitor T cell of embodiment 23, wherein the exogenous nucleic acid is integrated into a TRAC locus.

Embodiment 28: A composition comprising:
the engineered progenitor T cell of any one of embodiments 1 to 27; and
an artificial antigen presenting substrate (aAPS).

Embodiment 29: The composition of embodiment 28, wherein the aAPS comprises an antigen presenting molecule.

Embodiment 30: The composition of embodiment 29, wherein the antigen presenting molecule comprises an MHC class II molecule.

Embodiment 31: The composition of embodiment 29 or 30, wherein the engineered progenitor T cell expresses a TCR that associates with the antigen presenting molecule by a cognate antigen.

Embodiment 32: The composition of embodiment 28, wherein the aAPS comprises an artificial antigen presenting cell (aAPC), a bead, a particle, or a nanoparticle.

Embodiment 33: The composition of embodiment 28, wherein the aAPS comprises the aAPC.

Embodiment 34: The composition of embodiment 33, wherein the aAPC comprises a cell that is immobilized to a solid surface.

Embodiment 35: A method of treating a cancer in a subject diagnosed with cancer or a subject in need of immunosuppression, comprising administering to the subject a cell derived from the engineered progenitor T cell of any one of embodiments 1 to 27 or the engineered progenitor T cell contained in the composition of any one of embodiments 28-34.

Embodiment 36: The method of embodiment 35, wherein the cell comprises a Treg.

Embodiment 37: The method of embodiment 36, wherein the subject has an autoimmune disease or is a recipient of a cell or tissue transplant.

Embodiment 38: Use of the engineered progenitor T cell of any one of embodiments 1 to 37, in the manufacture of a medicament in treating a cancer or a subject in need of immunosuppression.

Embodiment 39: An engineered cell comprising:
a heterologous nucleic acid comprising a coding sequence for a lineage commitment factor that promotes differentiation of the engineered cell towards a CD4+ T cell, wherein the heterologous nucleic acid is integrated within the genome of the engineered cell in a sustained transcriptionally active payload region (STAPLR) or a sustained transgene expression loci (STEL).

Embodiment 40: The engineered cell of embodiment 39, wherein the heterologous nucleic acid is integrated in a STAPLR.

Embodiment 41: The engineered cell of embodiment 39, and wherein the STAPLR is selected from the group consisting of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene; and the intergenic region between the PA2G4 gene and the RPL41 gene.

Embodiment 42: The engineered cell of embodiment 41, wherein the STAPLR is the intergenic region between the PRDX1 gene and the AKR1A1 gene.

Embodiment 43: The engineered cell of any one of embodiments 39-42, wherein the heterologous nucleic acid is integrated at a location that is at least 100-5000 base pairs away from the nearest gene.

Embodiment 44: The engineered cell of embodiment 43, wherein the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

Embodiment 45: The engineered cell of embodiment 44, wherein the lineage commitment factor is ThPOK.

Embodiment 46: The engineered cell of any one of embodiments 39 to 45, wherein the heterologous nucleic acid further comprises an inducible promoter upstream of the lineage commitment factor.

Embodiment 47: The engineered cell of any one of embodiments 39 to 46, wherein the heterologous nucleic acid, when activated, results in at least a 2-fold increase in expression of the lineage commitment factor as compared to a comparable wild-type cell under similar conditions and thereby steers differentiation of the engineered cell into the CD4+ T cell.

Embodiment 48: The engineered cell of any one of embodiment 39 to 47, wherein the engineered cell further comprises a heterologous coding sequence for a second lineage commitment factor.

Embodiment 49: The engineered cell of embodiment 48, wherein the second lineage commitment factor, when activated, promotes differentiation of the CD4+ T cell towards a CD4+ T cell subtype.

Embodiment 50: The engineered cell of any one of embodiments 39 to 49, wherein the engineered cell comprises at least one of an embryonic stem cell, an induced pluripotent stem cell, a hematopoietic stem cell, a lymphoid progenitor cell, or a progenitor T cell.

Embodiment 51: The engineered cell of any one of embodiments 39 to 50, wherein the engineered cell is genetically modified to integrate an exogenous nucleic acid encoding a TCR within the genome of the engineered cell.

Embodiment 52: The engineered cell of embodiment 51, wherein the exogenous nucleic acid is integrated within a STEL.

Embodiment 53: The engineered cell of embodiment 52, wherein the STEL comprises a housekeeping gene.

Embodiment 54: The engineered cell of embodiment 53, wherein the housekeeping gene comprises GAPDH.

Embodiment 55: The engineered cell of embodiment 51, wherein the exogenous nucleic acid is integrated into a TCR alpha constant (TRAC) loci.

Embodiment 56: The engineered cell of any one of embodiments 39 to 55, wherein the engineered cell is further modified to reduce the expression of a competing lineage commitment factor.

Embodiment 57: The engineered cell of embodiment 56, wherein the competing lineage commitment factor comprises Runx3.

Embodiment 58: An engineered cell comprising a heterologous nucleic acid that, when activated, results in at least a 2-fold increase in expression of ThPOK as compared to a comparable wild-type cell under similar conditions, thereby promoting differentiation of the engineered cell towards a CD4+ T cell.

Embodiment 59: The engineered cell of embodiment 58, wherein the heterologous nucleic acid comprises a coding sequence for ThPOK.

Embodiment 60: The engineered cell of embodiment 58 or 59, wherein the heterologous nucleic acid further comprises an inducible promoter upstream of the coding sequence.

Embodiment 61: The engineered cell of any one of embodiments 58 to 59, wherein the heterologous nucleic acid is integrated into a STAPLR or STEL.

Embodiment 62: The engineered cell of embodiment 61, wherein the heterologous nucleic acid is integrated into a STAPLR.

Embodiment 63: The engineered cell of embodiment 62, wherein the STAPLR comprises one of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene; or
the intergenic region between the PA2G4 gene and the RPL41 gene.

Embodiment 64: The engineered cell of embodiment 63, wherein the STAPLR comprises the intergenic region between the PRDX1 gene and the AKR1A1 gene.

Embodiment 65: The engineered cell of any one of embodiments 58 to 64, wherein the heterologous nucleic acid is integrated at a location that is at least 100-5000 base pairs away from the nearest gene.

Embodiment 66: The engineered cell of any one of embodiments 58 to 60, wherein the heterologous nucleic acid is integrated in the genome at or near a gene that is specifically expressed in T cells.

Embodiment 67: The engineered cell of cany one of embodiments 58 to 60, wherein the heterologous nucleic acid is integrated in the genome at or near a TRAC locus.

Embodiment 68: The engineered cell of any one of embodiments 58 to 67, wherein the engineered cell further comprises at least one heterologous coding sequence for a lineage commitment factor that, when activated, promotes differentiation of the CD4+ T cell subtype.

Embodiment 69: The engineered cell of embodiment 68, wherein the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, or c-Myb.

Embodiment 70: The engineered cell of embodiment 69, wherein the engineered cell comprises one of an embryonic stem cell, an induced pluripotent stem cell, a hematopoietic stem cell, a lymphoid progenitor cell, or a progenitor T cell.

Embodiment 71: The engineered cell of any one of embodiments 58 to 70, wherein the engineered cell is a stem cell that is engineered to express a TCR that recognizes an autoantigen upon differentiation into a T cell.

Embodiment 72: A composition comprising the engineered cell of any one of embodiments 58 to 71 and further comprising an artificial antigen presenting substrate (aAPS).

Embodiment 73: The composition of embodiment 72, wherein the aAPS comprises an antigen presenting molecule.

Embodiment 74: The composition of embodiment 73, wherein the antigen presenting molecule comprises an MHC class II molecule.

Embodiment 75: The composition of embodiment 74, wherein the engineered cell comprises a T cell receptor (TCR) that binds with the MHC class II molecule of the artificial antigen presenting substrate by a cognate antigen.

Embodiment 76: The composition of embodiment 75, wherein the cognate antigen is linked to the MHC-class II molecule via a linker.

Embodiment 77: The composition of any one of embodiments 72 to 76, wherein the aAPS comprises an artificial antigen presenting cell (aAPC), a bead, a particle, or a nanoparticle.

Embodiment 78: A method of treating a cancer in a subject diagnosed with cancer or a subject in need of immunosuppression or suffering from autoimmune disease, comprising administering to the subject an engineered cell of any one of embodiments 58 to 71 or the engineered progenitor T cell contained in the composition of any one of embodiments 72 to 77.

Embodiment 79: The method of embodiment 78, wherein the subject is a recipient of a cell or tissue transplant.

Embodiment 80: Use of a cell derived from the engineered cell of any one of embodiments 58 to 77, in the manufacture of a medicament in treating a cancer or a subject in need of immunosuppression.

Embodiment 81: A method of making a CD4+ T cell, the method comprising:
activating a heterologous nucleic acid comprising a coding sequence for a lineage commitment factor within an engineered CD4−CD8− double negative (DN) cell or an engineered CD4+CD8+ double positive (DP) cell, wherein the activating results in an increased expression of said lineage commitment factor within the engineered DN cell or the engineered DP cell as compared to a comparable wild-type cell under similar conditions and thereby promotes the differentiation of the engineered DN cell or the engineered DP cell towards a CD4+ T cell.

Embodiment 82: The method of embodiment 81, wherein the activating occurs in the engineered DN cell.

Embodiment 83: The method of embodiment 81, wherein the activating occurs in the engineered DP cell.

Embodiment 84: The method of any one of embodiments 81 to 83, wherein the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

Embodiment 85: The method of embodiment 84, wherein the lineage commitment factor comprises ThPOK.

Embodiment 86: The method of any one of embodiments 81 to 85, wherein the heterologous nucleic acid comprises an inducible promoter upstream of the lineage commitment factor.

Embodiment 87: The method of embodiment 86, wherein the activating comprises activating the inducible promoter upstream of the coding sequence.

Embodiment 88: The method of embodiment 82, wherein the activating comprises contacting the DN cell with doxycycline or a derivative thereof.

Embodiment 89: The method of embodiment 83, wherein the activating comprises contacting the DP cell with doxycycline or a derivative thereof.

Embodiment 90: The method of any one of embodiments 81 to 89, wherein the DN or DP cell is derived from an induced pluripotent stem cell.

Embodiment 91: The method of any one of embodiments 81 to 90, further comprising overexpressing the lineage commitment factor at least 2-fold greater within the engineered DN or DP cell as compared to the comparable wild-type cell under similar conditions.

Embodiment 92: The method of embodiment 82, wherein the DN cell comprises a DN3 or DN4 cell.

Embodiment 93: A method of obtaining a population of cells enriched for CD4+ T cells, the method comprising:
obtaining a CD4−CD8− (DN) cells or CD4+CD8+(DP) cells; and
overexpressing at least one exogenous lineage commitment factor within the DN cells or DP cells as compared to a comparable wild-type cell under similar conditions to thereby produce a population of cells enriched for CD4+ T cells.

Embodiment 94: The method of embodiment 93, wherein the method is performed with DN cells.

Embodiment 95: The method of embodiment 93, wherein the method is performed with DP cells.

Embodiment 96: The method of any one of embodiments 93 to 95, further comprising contacting the DN cells or DP cells with a first artificial antigen presenting substrate (aAPS).

Embodiment 97: The method of embodiment 96, wherein the method further comprises contacting the DN cells or the DP cells with a second aAPS, wherein the second aAPS is different than the first aAPS.

Embodiment 98: A method of treating a cancer in a subject diagnosed with cancer or a subject in need of immunosuppression, comprising administering to the subject a cell derived from the CD4+ T cell made by the method of any one of embodiments 81 to 92 or a cell derived from the population of cells enriched for CD4+ T cells made by the method of any one of embodiments 93 to 97.

Embodiment 99: The method of embodiment 98, wherein the cell comprises a Treg.

Embodiment 100: A composition comprising:
a stem or progenitor T cell that expresses, or is capable of expressing upon differentiation into a T cell, an antigen recognition receptor; and
an artificial antigen presenting substrate (aAPS) comprising an immunomodulatory polypeptide.

Embodiment 101: The composition of embodiment 100, wherein the immunomodulatory polypeptide comprises an antigen presenting molecule.

Embodiment 102: The composition of embodiment 101, wherein the antigen presenting molecule comprises a major histocompatibility complex (MHC) molecule.

Embodiment 103: The composition of embodiment 102, wherein the MHC molecule comprises an MHC-class II molecule.

Embodiment 104: The composition of embodiment 103, wherein the interaction of the MHC-class II molecule or the cognate antigen with the antigen recognition receptor of the stem or progenitor T cell promotes differentiation towards a CD4+ T cell.

Embodiment 105: The composition of any one of embodiment 103 or embodiment 104, wherein the MHC-class II molecule comprises at least one of an HLA-DP molecule, an HLA-DM molecule, an HLA-DO molecule, an HLA-DQ molecule, or an HLA-DR molecule.

Embodiment 106: The composition of embodiment 105, wherein the MHC-class II molecule comprises the HLA-DR molecule.

Embodiment 107: The composition of any one of embodiments 100 to 106, wherein the aAPS comprises an artificial antigen presenting cell (aAPC).

Embodiment 108: The composition of embodiment 107, wherein the aAPC comprises a cell that is immobilized to a solid surface.

Embodiment 109: The composition of any one of embodiments 100 to 108, wherein the aAPS further comprises at least one co-stimulatory molecule.

Embodiment 110: The composition of embodiment 109, wherein the at least one co-stimulatory molecule comprises at least one of CD40, CD80, CD83, CD86, ICOS-L, CD58, or ICAM1.

Embodiment 111: The composition of embodiment 110, wherein the at least one co-stimulatory molecule comprises CD80 or ICAM1.

Embodiment 112: The composition of any one of embodiments 100 to 111, wherein the antigen recognition receptor comprises an artificial antigen receptor.

Embodiment 113: The composition of embodiment 112, wherein the artificial antigen receptor comprises a chimeric antigen receptor (CAR).

Embodiment 114: The composition of any one of embodiments 100 to 113, wherein the antigen recognition receptor comprises a T cell receptor (TCR).

Embodiment 115: The composition of any one of embodiments 100 to 114, wherein the aAPS comprises a Notch ligand.

Embodiment 116: The composition of embodiment 115, wherein the Notch ligand is human delta-like ligand 4 (DLL4).

Embodiment 117: The composition of any one of embodiments 100 to 116, wherein the stem or progenitor T cell comprises at least one of an embryonic stem cell, an induced pluripotent stem cell, a hematopoietic stem cell, a lymphoid progenitor cell, or a progenitor CD4+ T cell.

Embodiment 118: The composition of embodiment 117, wherein the stem cell is a hematopoietic stem cell derived from an induced pluripotent stem cell.

Embodiment 119: The composition of embodiment 118, wherein the induced pluripotent stem cell is derived from a T cell.

Embodiment 120: The composition of embodiment 119, wherein the T cell comprises a TCR that is specific to an autoantigen.

Embodiment 121: The composition of any one of embodiment 101 to 120, wherein the antigen presenting molecule is loaded with an antigen that is cognate to the antigen recognition receptor.

Embodiment 122: The composition of any one of embodiments 100 to 121, wherein the stem or progenitor T cell further comprises a heterologous nucleic acid comprising a coding sequence for a lineage commitment factor that, upon activation, promotes differentiation of the stem or progenitor T cell into a CD4+ T cell.

Embodiment 123: The composition of embodiment 122, wherein the heterologous nucleic acid is integrated within the genome of the stem or progenitor T cell in a sustained transcriptionally active payload region (STAPLR) or a sustained transgene expression locus (STEL).

Embodiment 124: The composition of embodiment 123, wherein the heterologous nucleic acid is integrated into the STAPLR.

Embodiment 125: The composition of embodiment 124, wherein the STAPLR comprises one of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene; or
the intergenic region between the PA2G4 gene and the RPL41 gene.

Embodiment 126: The composition of embodiment 125, wherein the STAPLR comprises the intergenic region between the PRDX1 gene and the AKR1A1 gene.

Embodiment 127: The composition of embodiment 122, wherein the heterologous nucleic acid is integrated into the genome of the stem or progenitor T cell at or near a gene that is specifically expressed in T cells.

Embodiment 128: The composition of embodiment 122, wherein the heterologous nucleic acid is integrated into the genome of the stem or progenitor T cell at or near a TRAC locus.

Embodiment 129: The composition of any one of embodiments 122 to 128, wherein the lineage commitment factor comprises at least one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

Embodiment 130: The composition of embodiment 129, wherein the stem or progenitor T cell further comprises at least two more heterologous lineage commitment factors.

Embodiment 131: The composition of embodiment 129 or embodiment 130, wherein the lineage commitment factor comprises ThPOK.

Embodiment 132: The composition of any one of embodiments 122 to 131, wherein the stem or progenitor T cell further comprises a second lineage commitment factor that, when activated, promotes differentiation towards at a CD4+ T cell subtype.

Embodiment 133: The composition of any one of embodiments 122 to 132, wherein the heterologous nucleic acid, when activated, results in at least a 2-fold increase in expression of the lineage commitment factor as compared to a comparable wild-type cell under similar conditions and thereby promotes differentiation of the stem or progenitor T cell into the CD4+ T cell.

Embodiment 134: A method of treating a cancer in a subject diagnosed with cancer or a subject in need of immunosuppression, comprising administering to the subject a cell derived from the engineered cell or progenitor T cell contained in the composition of any one of embodiments 100 to 133.

Embodiment 135: The method of embodiment 134, wherein the cell derived from the stem or progenitor T cell is a Treg.

Embodiment 136: The method of embodiment 134 or embodiment 135, wherein the subject has an autoimmune disease or is a recipient of a transplant.

Embodiment 137: Use of the stem or progenitor T cell contained in the composition of any one of embodiments 100 to 133, in the manufacture of a medicament in treating a cancer or a subject in need of immunosuppression.

Embodiment 138: An artificial antigen presenting substrate (aAPS) comprising an antigen presenting molecule linked to an antigen by a peptide.

Embodiment 139: The aAPS of embodiment 138, wherein the antigen presenting molecule comprises a major histocompatibility complex (MHC) molecule.

Embodiment 140: The aAPS of embodiment 139, wherein the MHC molecule comprises an MHC-class II molecule.

Embodiment 141: The aAPS of any one of embodiments 138 to 140, wherein the aAPS further comprises at least one co-stimulatory molecule or a Notch ligand.

Embodiment 142: The aAPS of any one of embodiments 138 to 141, wherein the aAPS comprises an artificial antigen presenting cell (aAPC).

Embodiment 143: The aAPS of embodiment 142, wherein the aAPC comprises a cell that is immobilized to a solid surface.

Embodiment 144: The aAPS of any one of embodiments 138 to 143, wherein the MHC-class II molecule comprises at least one of an HLA-DP molecule, an HLA-DM molecule, an HLA-DO molecule, an HLA-DQ molecule, or an HLA-DR molecule.

Embodiment 145: The aAPS of embodiment 144, wherein the MHC-class II molecule comprises an HLA-DR molecule.

Embodiment 146: The aAPS of any one of embodiments 141 to 14, wherein the aAPS comprises the at least one co-stimulatory molecule and the at least one co-stimulatory molecule comprises at least one of CD40, CD80, CD86, ICOS-L, CD58, or ICAM1.

Embodiment 147: The aAPS of embodiment 146, wherein the co-stimulatory molecule comprises CD80 or ICAM1.

Embodiment 148: The aAPS of any one of embodiments 141 to 147, wherein the aAPS comprises the Notch ligand and the Notch ligand comprises DLL4.

Embodiment 149: The aAPS of any one of embodiments 138 to 140, wherein the aAPS is engineered to secrete one or more cytokines or growth factors.

Embodiment 150: The aAPS of embodiment 149, wherein the one or more cytokines or growth factors are selected to influence cell fate of a target cell into a CD4+ T cell or regulatory T cell.

Embodiment 151: The aAPS of embodiment 149 or embodiment 150, wherein the one or more cytokines or growth factors comprises TGF-b.

Embodiment 152: A composition comprising the aAPS of any one of embodiments 41 to 54 and cell culture media.

Embodiment 153: The composition of embodiment 152, further comprising a progenitor T cell that expresses, or is capable of expressing upon differentiation into a T cell, an antigen recognition receptor.

Embodiment 154: The composition of embodiment 153, wherein the antigen recognition receptor comprises a TCR.

Embodiment 155: The composition of embodiment 154, wherein the antigen of the aAPS is bound by the TCR of the progenitor T cell.

Embodiment 156: A method of producing a CD4+ T cell, the method comprising: combining a progenitor T cell that expresses an antigen recognition receptor, or is capable of expressing said antigen recognition receptor upon differentiation into a T cell, with an artificial antigen presenting substrate (aAPS);
differentiating the progenitor T cell towards a CD4+ T cell; and
contacting the antigen presenting molecule of the aAPS with the antigen recognition receptor of the T cell thereby promoting differentiation of the stem or progenitor cell towards a CD4+ T cell.

Embodiment 157: The method of embodiment 156, wherein the aAPS comprises an antigen presenting molecule.

Embodiment 158: The method of embodiment 156 or embodiment 157, wherein the aAPS comprises an artificial antigen presenting cell (aAPC).

Embodiment 159: The method of embodiment 158, wherein the aAPC comprises a cell that is immobilized to a solid surface.

Embodiment 160: The method of any one of embodiments 157 to 159, wherein the antigen presenting molecule comprises a major histocompatibility complex (MHC).

Embodiment 161: The method of embodiment 160, wherein the MHC comprises an MHC-class II molecule.

Embodiment 162: The method of embodiment 161, wherein the MHC-class II molecule comprises at least one of an HLA-DP molecule, an HLA-DM molecule, an HLA-DO, an HLA-DQ molecule, or an HLA-DR molecule.

Embodiment 163: The method of embodiment 162, wherein the MHC-class II molecule is an HLA-DR molecule.

Embodiment 164: The method of any one of embodiments 158 to 163, wherein the aAPS further comprises a co-stimulatory molecule.

Embodiment 165: The method of embodiment 164, wherein the co-stimulatory molecule comprises at least one of CD40, CD80, CD86, ICOS-L, CD58, ICAM1.

Embodiment 166: The method of any one of embodiments 158 to 165, wherein the aAPS further comprises a Notch ligand.

Embodiment 167: The method of embodiment 166, wherein the Notch ligand comprises DLL4.

Embodiment 168: The method of any one of embodiments 158 to 167, wherein the combining comprises:
adhering the aAPS to a surface of a tissue culture dish; and
adding the progenitor T cell to the tissue culture dish comprising the aAPS.

Embodiment 169: The method of any one of embodiments 158 to 168, further comprising contacting the progenitor T cell with a second aAPS, wherein the second aAPS is different from the first aAPS.

Embodiment 170: The method of embodiment 169, wherein the second aAPS comprises an antigen presenting molecule.

Embodiment 171: The method of embodiment 170, wherein the second aAPS, but not the aAPS, comprises the antigen presenting molecule.

Embodiment 172: The method of any one of embodiments 158 to 171, wherein the progenitor T cell comprises a heterologous nucleic acid comprising a coding sequence for a lineage commitment factor.

Embodiment 173: The method of embodiment 172, further comprising activating the heterologous nucleic acid in the progenitor T cell, wherein the activating results in the stem or progenitor T cell expressing the lineage commitment factor at a higher level than a comparable wild-type stem or progenitor T cell.

Embodiment 174: The method of embodiment 172 or embodiment 173, wherein the progenitor T cell comprises a CD4+CD8+DP phenotype.

Embodiment 175: The method of embodiment 172 or embodiment 173, wherein the progenitor T cell comprises a CD4−CD8− DN phenotype.

Embodiment 176: The method of any one of embodiments 172 to 175, wherein the heterologous nucleic acid is integrated within the genome of the progenitor T cell in a sustained transcriptionally active payload region (STAPLR) or a sustained transgene expression loci (STEL).

Embodiment 177: The method of embodiment 176, wherein the STAPLR comprises one of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene; or
the intergenic region between the PA2G4 gene and the RPL41 gene.

Embodiment 178: The method of embodiment 177, wherein the STAPLR is the intergenic region between the PRDX1 gene and the AKR1A1 gene.

Embodiment 179: The method of embodiment 178, wherein the lineage commitment factor comprises at least one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

Embodiment 180: The method of embodiment 179, wherein the lineage commitment factor comprises ThPOK.

Embodiment 181: The method of any one of embodiments 171 to 180, wherein the progenitor T cell is further modified to comprise a coding sequence for a second lineage commitment factor that, when activated, promotes differentiation towards a CD4+ T cell subtype.

Embodiment 182: The method of embodiment 181, wherein the heterologous nucleic acid, when activated, results in at least a 2-fold increase in expression of the lineage commitment factor as compared to the comparable wild-type stem or progenitor cell under similar conditions and thereby steers differentiation of the progenitor T cell into the CD4+ T cell.

Embodiment 183: A method of treating a cancer in a subject diagnosed with cancer or a subject in need of immunosuppression, comprising administering to the subject a cell obtained from the method of any one of embodiments 158 to 182.

Embodiment 184: The method of embodiment 183, wherein the cell obtained from the stem or progenitor T cell is a regulatory T cell.

Embodiment 185: The method of embodiment 183 or embodiment 184, wherein the subject has an autoimmune disease or is a recipient of a transplant.

Embodiment 186: Use of a cell obtained from the method of any one of embodiments 158 to 182, in the manufacture of a medicament in treating a cancer or subject in need of immunosuppression.

### EXAMPLES

### Example 1: Stem Cells with Inducible Lineage Commitment Factors

This example describes experiments that were performed to generate stem cells that harbor an exemplary inducible lineage commitment factor (ThPOK), which upon activation, can facilitate the formation of CD4+ T cells. To allow for controlled and robust expression of the exemplary lineage commitment factor, a dual component doxycycline-inducible rtTA/TRE system was used (e.g., as described in U.S. Pat. No. 9,127,283, which is incorporated by reference).

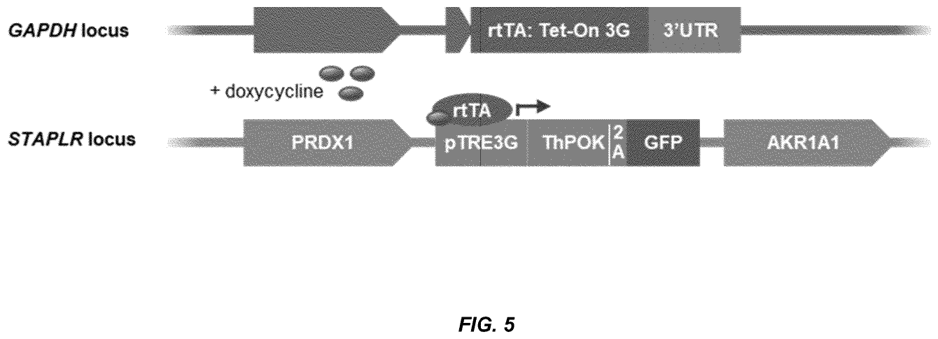

FIG. 5 is a diagram illustrating an inducible expression system. To employ the expression system in ThPOK-edited cells, an exogenous nucleic acid encoding a doxycycline (dox)-responsive, transcriptional regulator protein (rtTA) was integrated into a STEL site located at the 3′ UTR of the GAPDH locus using a Cas ribonucleoprotein. The Cas ribonucleoprotein was nucleofected into the cells with a STEL targeting construct comprising left and right homology arms designed to facilitate in-frame integration of the rtTA upstream of the STOP codon of GAPDH. Integration of the construct upstream of the STOP codon allows for constitutive expression of rtTA from the GAPDH gene promoter.

A construct encoding ThPOK and a green fluorescent protein (GFP) downstream of a pTRE3G promoter was integrated into a STAPLR comprising the intergenic region between genes PRDX1 and AKR1A1. The ThPOK and GFP coding sequences are separated by a 2A self-cleaving peptide sequence, which allows ThPOK and GFP proteins to be processed separately in the cell despite originating from shared mRNA transcripts. As described above, the addition of dox to cell cultures enables binding of the rtTA to the pTRE3G promoter resulting in robust expression of ThPOK and GFP. To validate target integration of the first and second nucleic acids junction PCR was performed on the ThPOK-edited iPSCs.

After validating site-specific integration, experiments were conducted to validate inducible expression of ThPOK from the STAPLR site by treating cells with dox and monitoring for GFP expression. Briefly, ThPOK-edited iPSCs were plated into cell culture dishes. One portion of cell culture dishes were treated with 2 μg/mL doxycycline (dox) for 48 hours. As a negative control, a second portion of the cell culture dishes were left untreated. Samples of treated and untreated cells were harvested and stained for cell surface antigen TRA-1-60, which is associated with pluripotency, to assess cell stemness. Samples from cells corresponding to dox treated and untreated cultures were then subjected to FACS analysis.

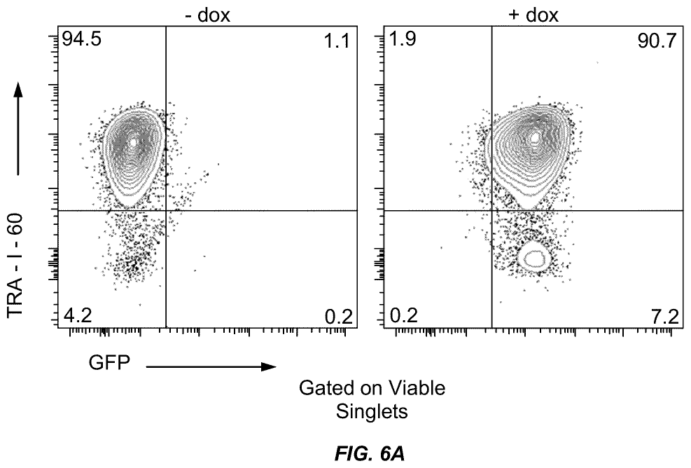

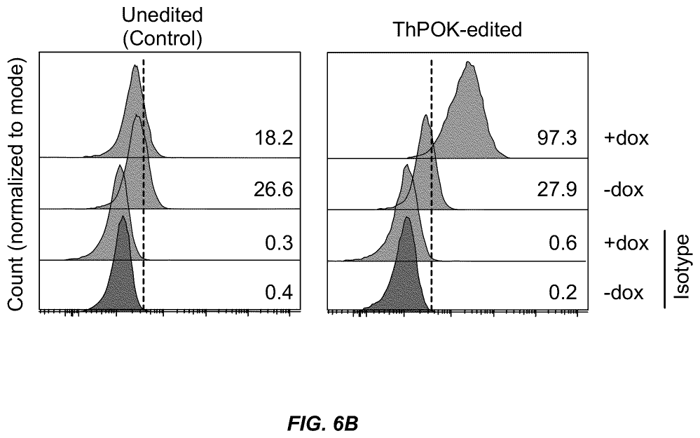

FIGS. 6A and 6B show exemplary experimental results confirming inducible ThPOK expression from a STAPLR in ThPOK-edited iPSCs. In particular, FIG. 6A shows flow cytometry plots of cells with ThPOK:GFP integrated into a STAPLR of iPSCs untreated (left panel) and treated (right panel) with dox. The treated cells were treated with 2 μg/mL dox for 48 hours. For FACS analysis, the cells were gated on viable singlets. In the FACS profiles, the X-axis corresponds to GFP expression (indicative of ThPOK expression). The Y-axis corresponds to the expression of pluripotency marker TRA-I-60. These results show that ThPOK is induced (i.e., 97.9% of cells are GFP positive) by the addition of dox to cell cultures. These results also suggest that induction of ThPOK may drive cell differentiation as a higher population of cells treated with dox are negative for pluripotency marker TRA-1-60 (compare 7.2 percent in dox treated cells with the untreated cells).

FIG. 6B shows exemplary histograms of control (left panel) and ThPOK-edited iPSCs (right panel) analyzed by flow cytometry for ThPOK expression (see X-axis). Cell cultures of control and ThPOK-edited iPSCs were treated (top histograms) or, as a control, untreated (second histograms from top) with 2 μg/mL of dox for 48 hours. The cultured cells were subsequently stained with a human monoclonal antibody against ThPOK and subjected to flow cytometry analysis. These results show that ~97.3 percent of ThPOK-edited cells treated with dox express ThPOK within 48 hours after treatment.

To confirm genomic integrity of the ThPOK-edited iPSCs, the cells were subjected to cytogenetic analysis (karyotyping).

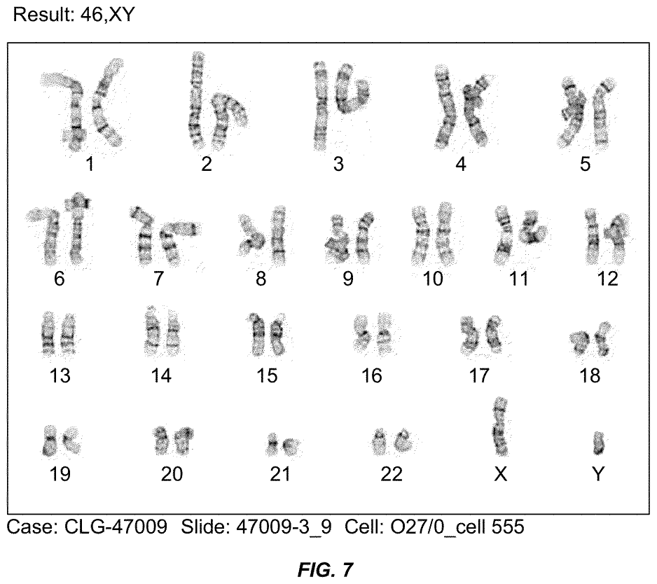

FIG. 7 shows exemplary cytogenetic data of ThPOK-edited iPSCs. The cytogenetic analysis was performed on twenty G-banded metaphase ThPOK-edited iPSCs. These data demonstrate that iPSCs genetically modified by the integration of an inducible ThPOK transgene maintain a normal karyotype.

To determine whether ThPOK-edited iPSCs maintain stemness, these cells were cultured for ~10-13 passages before being subjected to an immunoassay in which the cells were stained with antibodies against known pluripotency markers: SSEA3, SOX2, NANOG, OCT4, TRA-1-60, and SSEA4. After staining, the cells were subjected to FACS analysis.

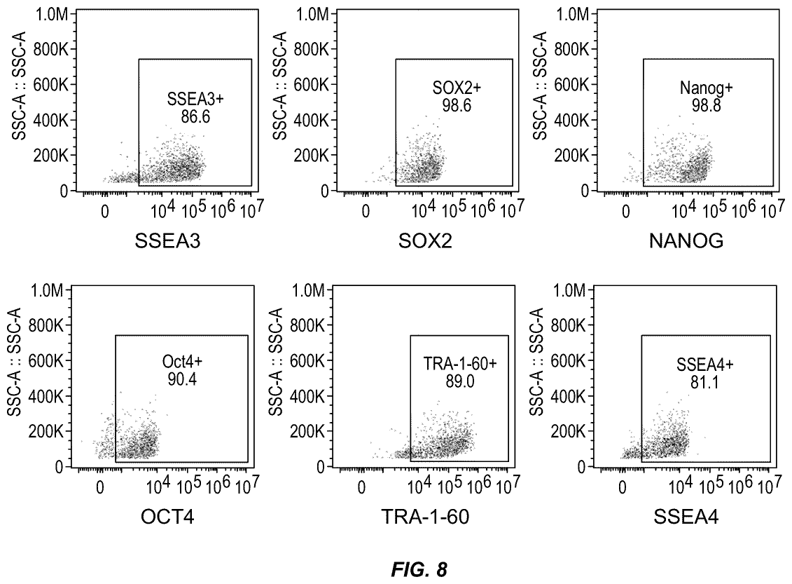

FIG. 8 shows exemplary flow cytometry data confirming the ThPOK-edited iPSCs maintain expression of pluripotency markers. In particular, FIG. 8 shows six flow cytometry plots of ThPOK-edited iPSCs stained for one of SSEA3, SOX2, NANOG, OCT4, TRA-1-60, or SSEA4 as indicated. The data show that 86.6 percent of ThPOK-edited iPSCs were positive for SSEA3; 98.6 percent of ThPOK-edited iPSCs were positive for SOX2; 98.8 percent of ThPOK-edited iPSCs were positive for NANOG; 90.4 percent of ThPOK-edited iPSCs were positive for OCT4; 89.1 percent of ThPOK-edited iPSCs were positive for TRA-1-60; and 81.1 percent of ThPOK-edited iPSCs were positive for SSEA4.

### Example 2: Induction of ThPOK Expression Results in Differentiation of ThPOK-Edited iPSCs

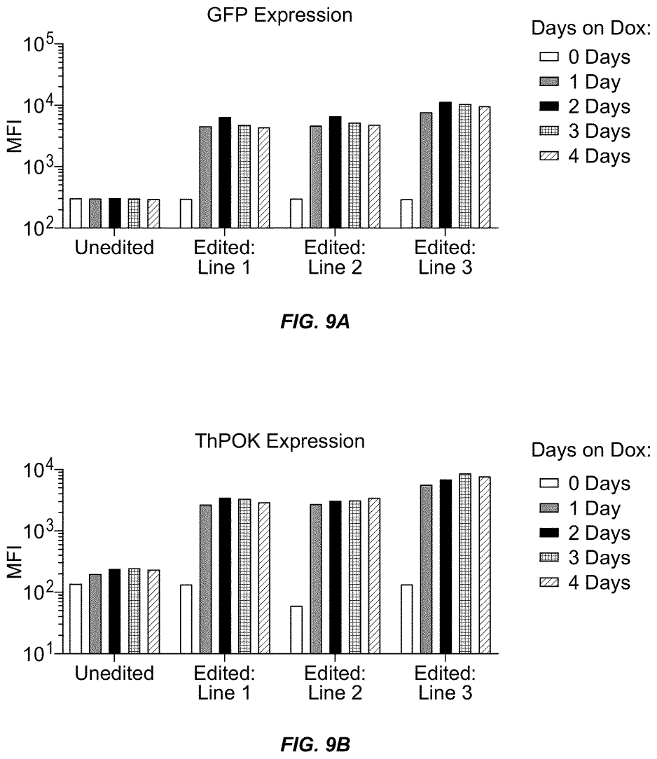

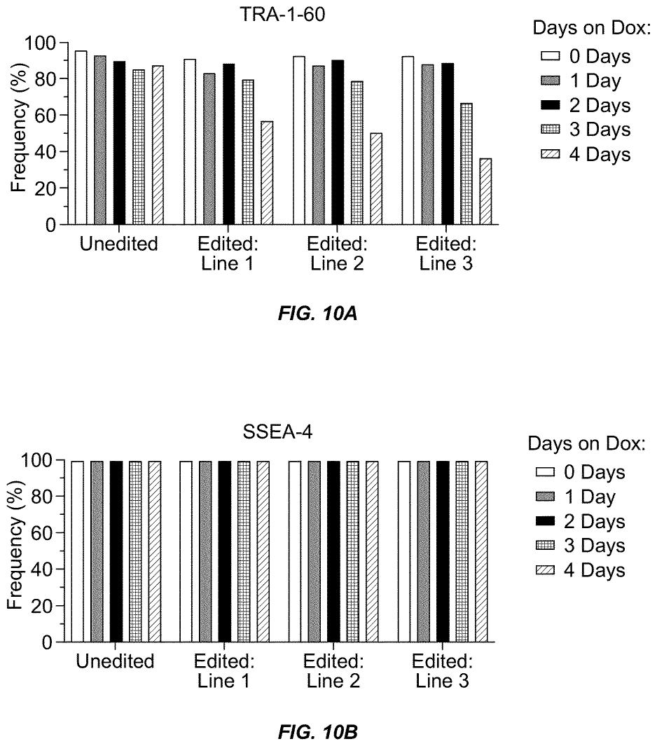

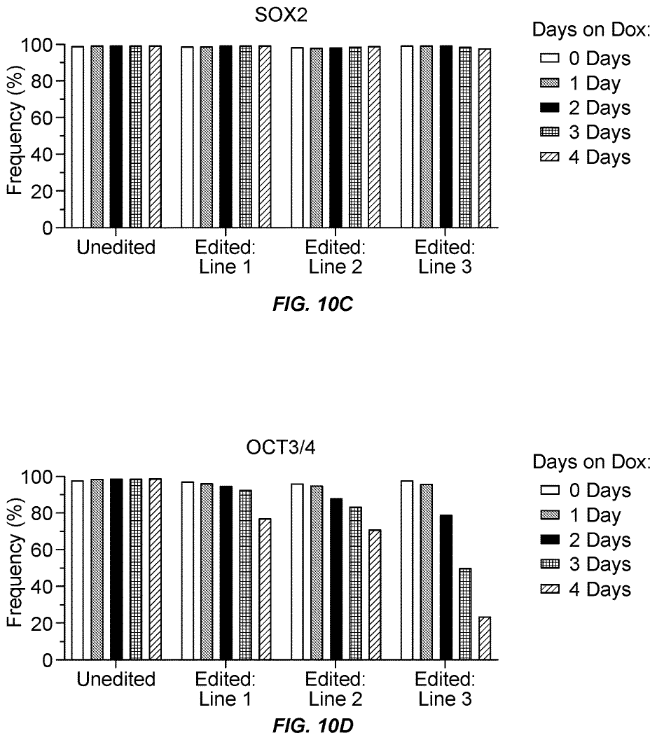

This example describes experimental work demonstrating functionality of the inducible ThPOK protein and shows biological consequences of inducing ThPOK expression in iPSCs. For this experiment, ThPOK-edited iPSCs from Example 1 were used. With the ThPOK-edited iPSCs, ThPOK was induced by the addition of 2 μg/mL dox for 1, 2, 3, or 4 days. After induction, the cells were stained with antibodies against the markers (i.e., ThPOK, TRA-60, SSEA4, SOX2, OCT3/4) indicated in FIGS. 9 and 10 below and subjected to flow cytometry. Cells were gated on viable singles and fluorescent cells were counted.

FIGS. 9A and 9B are exemplary bar graphs from flow cytometry data showing GFP (FIG. 9A) and ThPOK expression (FIG. 9B) of unedited and ThPOK-edited cells. In particular, FIG. 9A shows GFP expression of different cell populations (one unedited cell line, three ThPOK-edited cell lines) following 0, 1, 2, 3, or 4 days (in order) of dox treatment as described above. The different cell populations include unedited cells and three edited cell lines. Each bar represents GFP expression, as measured by flow cytometry, following a different time of treatment (i.e., shown in order from 0, 1, 2, 3, or 4 days) with dox. The data show GFP is rapidly upregulated in edited cell populations upon addition of dox as soon as 1 day following treatment. The different cell populations of FIG. 9A were stained with antibodies against ThPOK. FIG. 9B shows ThPOK expression corresponds with GFP expression, e.g., ThPOK expression is rapidly upregulated upon addition of dox for 1 day.

FIGS. 10A-10D are exemplary bar graphs taken from flow cytometry data of the cell populations described above (i.e., unedited and ThPOK edited cells treated with dox for 0, 1, 2, 3, or 4 days) demonstrating induction of ThPOK in iPSCs results in a loss of certain pluripotency makers, i.e., TRA-1-60 and Oct 3/4. FIG. 10A shows bar graphs from flow cytometry data after staining cells with an antibody against TRA-1-60. These results show that after two days of dox treatment (days 3 and 4), the number of cells that are positive for pluripotency marker TRA-1-60 is reduced. FIG. 10B shows bar graphs on the cell populations after staining the cells with an antibody against SSEA-4. These results show that the number of cells positive for SSEA-4 remains constant. FIG. 10C shows bar graphs on the cell populations described above after staining the cells with antibodies against Sox2. These results show that the number of cells positive for Sox2 remains constant. FIG. 10D shows bar graphs on the cell populations described above after staining the cells with antibodies against Oct3/4. These results show that the number of cells positive for Oct3/4 is rapidly reduced upon treatment with dox. Taken together, these results show induction of ThPOK by the addition of dox results in a loss of pluripotency markers TRA-1-60 and Oct3/4 demonstrating that the inducible ThPOK is functional and when expressed in iPSCs causes changes in cell type.

To further evaluate the impact of induced ThPOK expression, cell morphology was evaluated in cells treated or untreated with dox.

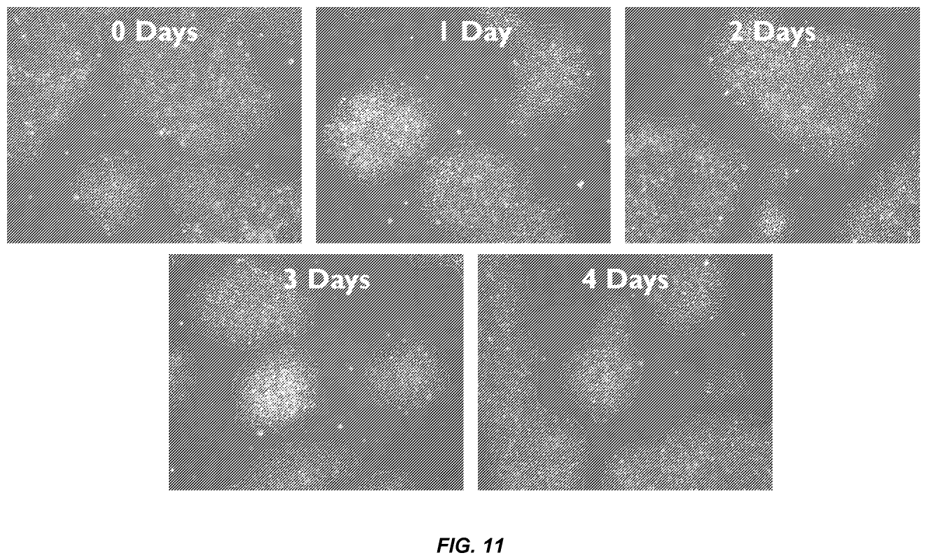

FIG. 11 shows exemplary images of unedited iPSCs during dox treatment. In particular, shown are panels of microscopic images of iPSCs treated with dox for 0, 1, 2, 3, or 4 days, as indicated. The images show the iPSCs retain an iPSC-like phenotype. That is, the iPSCs retain an appearance of compact colonies having distinct borders and well-defined edges and are comprised of cells with a large nucleus with less cytoplasm.

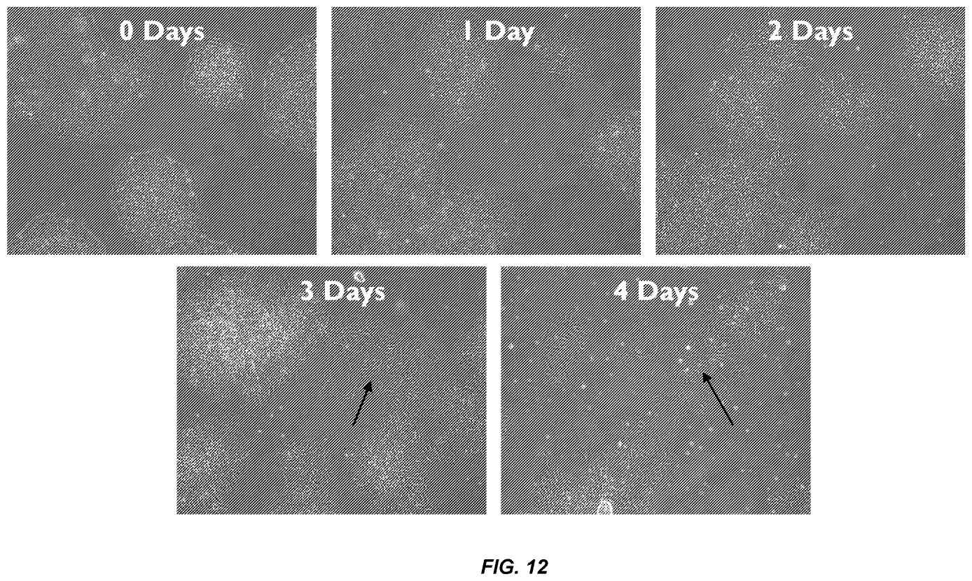

FIG. 12 shows exemplary images of ThPOK-edited iPSCs during dox treatment. In particular, shown are panels of microscopic images of ThPOK-edited iPSCs treated with dox for 0, 1, 2, 3, or 4 days, as indicated. The images show that portions of the populations of ThPOK-edited iPSCs lose their iPSC-like morphology after two days of ThPOK induction. Exemplary portions of ThPOK-edited cells that have lost their stem cell morphology are identified by black arrows. The cells that have lost their iPSC-like morphology exhibit epithelial-like morphology, i.e., individual cells separate from compact colonies and have a high cytoplasm to nucleus ratio.

### Example 3: Premature Induction of ThPOK Expression can Inhibit T Cell Development

These experiments demonstrate that the temporal kinetics of ThPOK expression, i.e., the timing of ThPOK induction, can influence differentiation of stem or progenitor T cells into CD4+ T cells. In particular, these experiments demonstrate that premature induction of ThPOK expression (e.g., induction in iPSCs) can inhibit the formation of CD4+ T cells. These experiments also demonstrate that the DN4 or DP development stage may represent a more optimal time window for inducing ThPOK to thereby steer differentiation towards a CD4+ T cell fate.

In the experiments described below, iPSCs were differentiated into CD34+ stem cells (HSPCs) following the differentiation procedure described above and shown in FIG. 3.

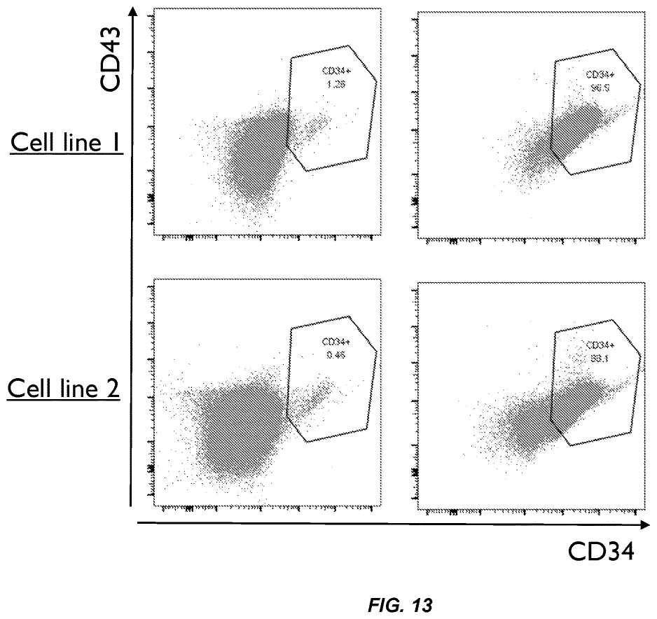

FIG. 13 shows exemplary flow cytometry data of iPSCs (unedited) following CD34+ differentiation. In particular, FIG. 13 shows CD43 (y-axis) and CD34 (x-axis) expression data from two exemplary cell lines collected following differentiation of unedited iPSCs to CD34+ cells (left panel), which can be enriched for by magnetic enrichment using antibodies against CD34 (right panel). Following enrichment, cell populations in which more than 88 percent of the cells were positive for CD34 were placed in cell culture dishes and induced to differentiate into T cells according to the procedure described in FIG. 4.

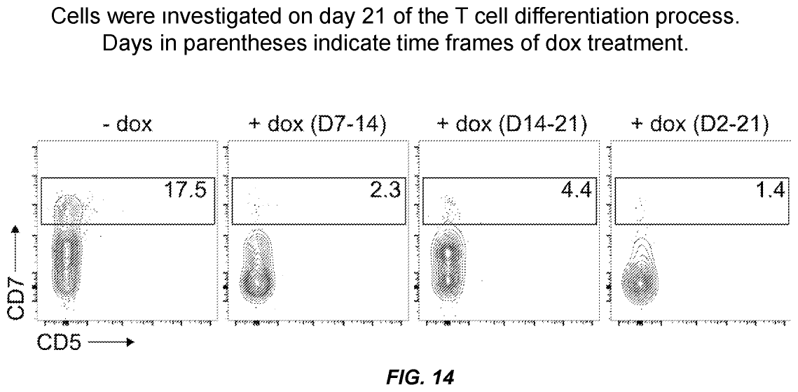

FIGS. 14 & 15 are exemplary experimental results demonstrating that premature induction of ThPOK can inhibit T cell development.

FIG. 14 shows exemplary flow cytometry data demonstrating premature induction of ThPOK (e.g., before or at day 14 of T cell differentiation) can result in reduced expression of CD7. In particular, FIG. 14 shows flow cytometry profiles of ThPOK-edited cells in which ThPOK had been induced during T cell differentiation during the time window indicated, e.g., between days 2-21; between days 7-14; between days 14-21, of T cell differentiation. The gene-edited cells were differentiated as described above. After day 21 of the T cell differentiation protocol, cells were stained for CD5 and CD7. These data show premature induction of ThPOK (e.g., between days 2-14) resulted in reduced expression of CD7, which is required for T cell development.

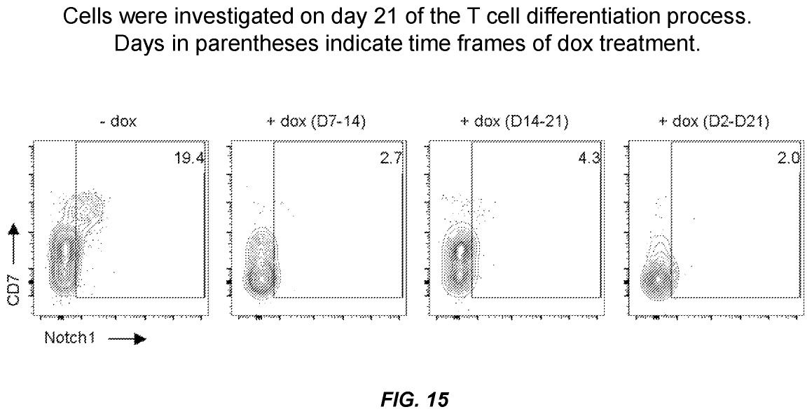

FIG. 15 shows flow cytometry data demonstrating premature induction of ThPOK (e.g., before day 14) results in reduced Notch1 expression. In particular, FIG. 15 shows exemplary flow cytometry profiles of ThPOK-edited cells in which ThPOK had been induced during T cell differentiation during the time window indicated, e.g., between days 2-21; between days 7-14; between days 14-21, of T cell differentiation. The data show that in samples of cell cultures not treated with dox, populations of CD7+ cells are positive for Notch1 expression; however, in samples from cultures treated with dox (e.g., during days 2-21; during days 7-14; or during days 14-21) CD7+ cells are negative for Notch1. Since Notch signaling is important for T cell development, these data show premature induction of ThPOK inhibits T cell differentiation.

Taken together, these data show premature induction of ThPOK (e.g., before DP) can inhibit CD4+ T cell development. Based on these data, it was hypothesized that DN4 or DP cells may represent an optimal window for induction of ThPOK for the generation of CD4+ T cells from stem or progenitor T cells.

### Example 4: Induction of ThPOK in DP T Cells Promotes the Efficient Generation of CD4+ T Cells

Gene edited iPSCs are differentiated into CD34+ cells according to the procedure described above and shown in FIG. 3. The CD34+ cells are optionally enriched and then induced to differentiate into T cells according to the procedure described above and shown in FIG. 4. After 7-14 days, the cells are stained for surface markers CD7, CD4, and CD8a. Cells positive for CD7 as well as positive for both CD4 and CD8a (CD4 CD8 DP), are plated into tissue culture dishes and ThPOK is induced in one portion of the tissue culture dishes by addition of dox to the cell cultures. Following 2-7 days, cells from dox treated and untreated cultures are harvested and stained for CD7, CD5, CD4, and CD8a, and subsequently subjected to flow cytometry analysis. Populations showing a higher percentage of CD4+CD8α− (CD4SP) T cells in cultures from CD4 CD8 DP cells in which ThPOK expression was induced as compared to cultures from cells in which ThPOK expression was not induced will show that induction of THPOK expression at a later stage of progenitor T cell differentiation is preferred.

CD4+ T cells generated by the induction of ThPOK at the DP stage are further subjected to scRNAseq for assessing the transcriptome of the cells, which are compared to the transcriptome of naturally derived CD4+ T cells. Comparable results between assayed populations would indicate substantial conformity to naturally derived CD4+ T cells.

### Example 5: IPSCs Engineered to Express an Autoantigen Specific TCR

This example describes experiments that were performed to generate iPSCs that, upon differentiation, express a TCR from a STEL. Integration of the TCR at the STEL (i.e., GAPDH locus) was useful to ensure high efficiency of genome editing and robust expression of the TCR upon T cell formation.

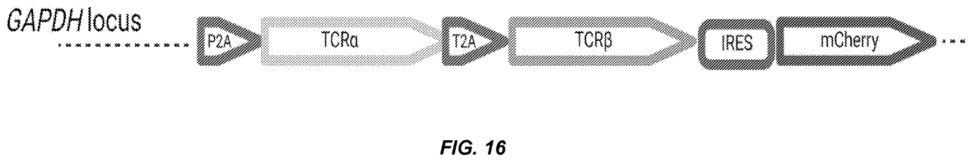

FIG. 16 shows a construct that was used to integrate a TCR into an iPSC. Briefly, iPSCs were nucleofected with a Cas RNP and a STEL targeting construct comprising the autoantigen TCR alpha and beta chains separated by a T2A sequence. The construct further included a fluorescent protein (mCherry). The construct also included left and right homology arms which facilitated site-specific integration of the construct upstream of the STOP codon of GAPDH. Expression of mCherry was used to identify cells carrying the autoantigen specific TCR, which are referred to herein as GAPDH-TCR engineered iPSCs.

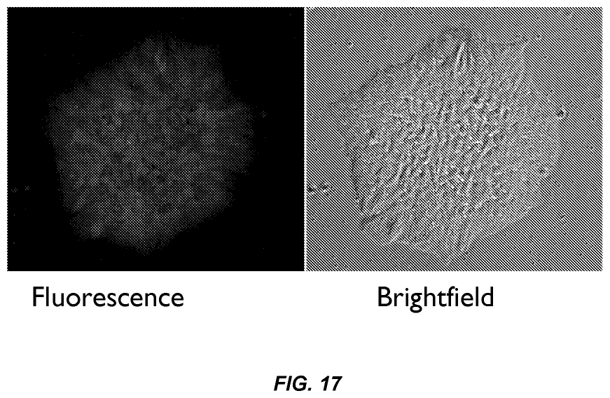

FIG. 17 shows exemplary microscope images of GAPDH-TCR engineered iPSCs. The left panel is a fluorescent image of a colony of GAPDH-TCR engineered iPSCs expressing mCherry, which demonstrates the TCR construct is integrated and expressed in the iPSCs. The right panel is a brightfield image of the cell colony from the right panel.

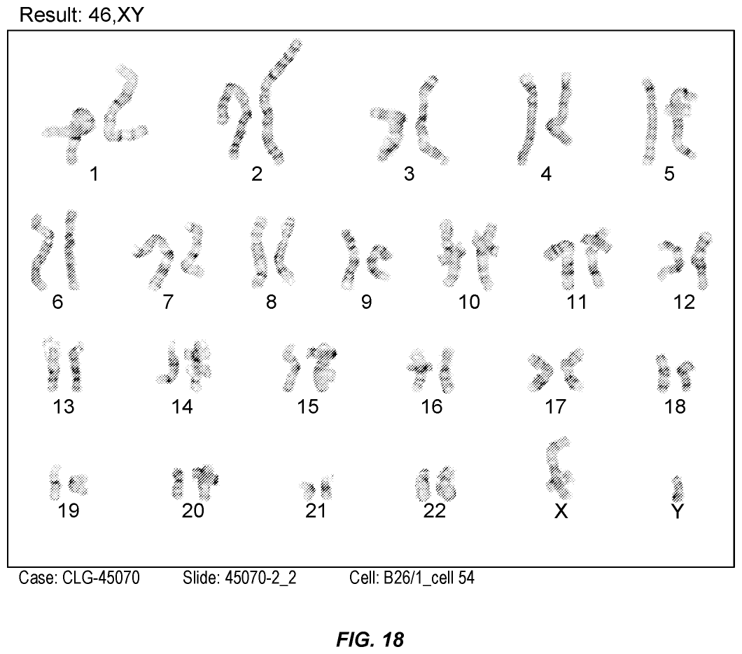

FIG. 18 shows exemplary cytogenetic data of GAPDH-TCR engineered iPSCs. The cytogenetic analysis was performed on G-banded metaphase GAPDH-TCR engineered iPSCs. The data demonstrate that the GAPDH-TCR engineered iPSCs exhibit a normal karyotype.

To determine whether GAPDH-TCR engineered iPSCs maintain pluripotency, two lines of GAPDH-TCR engineered iPSCs were each passaged for ~10 passages and stained with antibodies against pluripotency markers, i.e., SSEA-3, Oct3/4, and TRA-1-60, and subjected to FACS analysis.

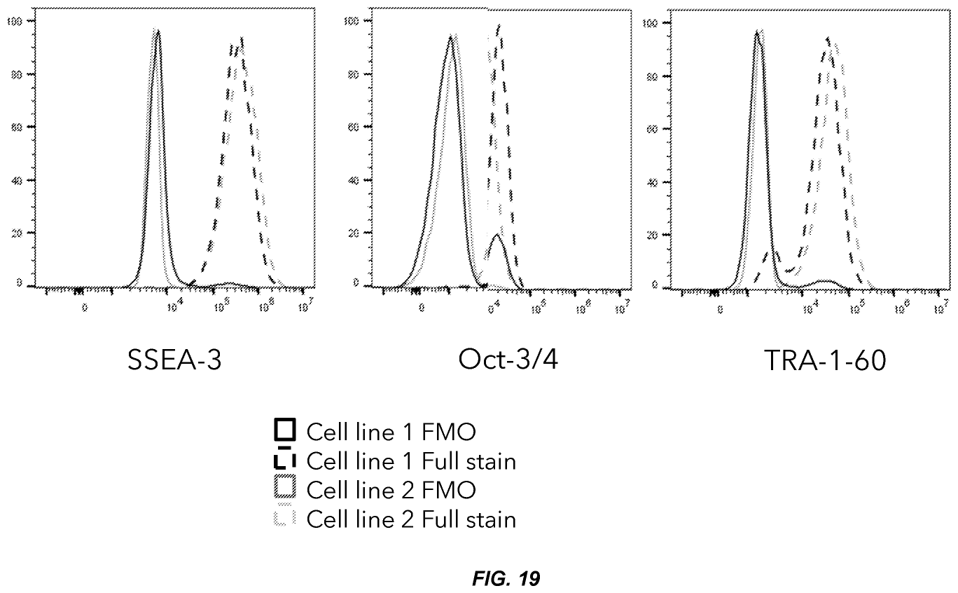

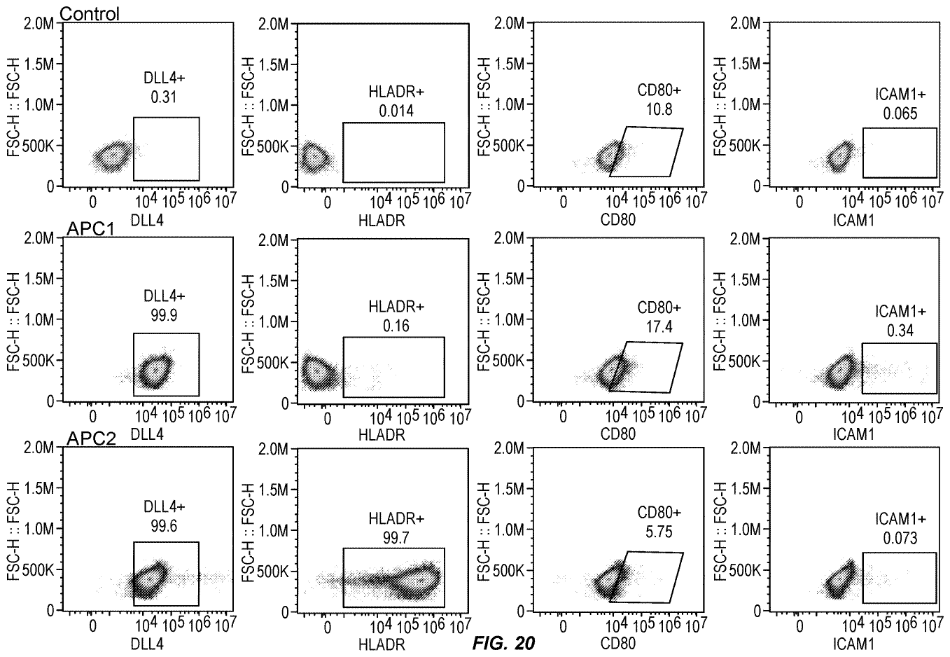

FIG. 19 shows exemplary histograms from a FACS analysis of GAPDH-TCR engineered iPSCs stained for pluripotency markers. In particular, these data show GAPDH-TCR engineered iPSCs that were unstained (FMO) or stained (Full stain) for SSEA-3, Oct 3/4, and TRA-1-60. These data demonstrate GAPDH-TCR engineered iPSCs were positive for pluripotency markers thus demonstrating the GAPDH-TCR engineered iPSCs maintain pluripotency.

### Example 6: Generating Artificial Antigen Presenting Substrates (aAPSs)

This example describes artificial antigen presenting substrates (aAPSs) in the form of artificial antigen presenting cells (aAPCs) that were generated to present cognate antigens to antigen recognition receptor (TCRs) of developing T cells in order to steer development towards a CD4+ T cell fate.

Briefly, adherent OP9 cells were transduced with nucleic acid constructs encoding DLL4, HLA-class II molecule (HLA-DR), and co-stimulatory molecules CD80 and ICAM1. The nucleic acid encoding HLA-DR further encoded a cognate peptide which was linked to the HLA-DR via a linker sequence. The constructs were transduced into the cells via lentiviral transduction.

FIG. 20 shows FACS profiles of aAPCs expressing DLL4, HLA-DR, CD80, and ICAM1. In particular, shown are FACS profiles of control aAPCs (unstained), aAPCs transduced with DLL4 only (APC1); aAPCs transduced with DLL4 and HLA-DR (APC2); aAPCs transduced with DLL4, HLA-DR, and CD80 (APC3); aAPCs transduced with DLL4, HLA-DR, CD80, and ICAM1 (APC4).

### Example 7: Generating CD4+ T Cells from Stem Cells Using Artificial Antigen Presenting Substrates

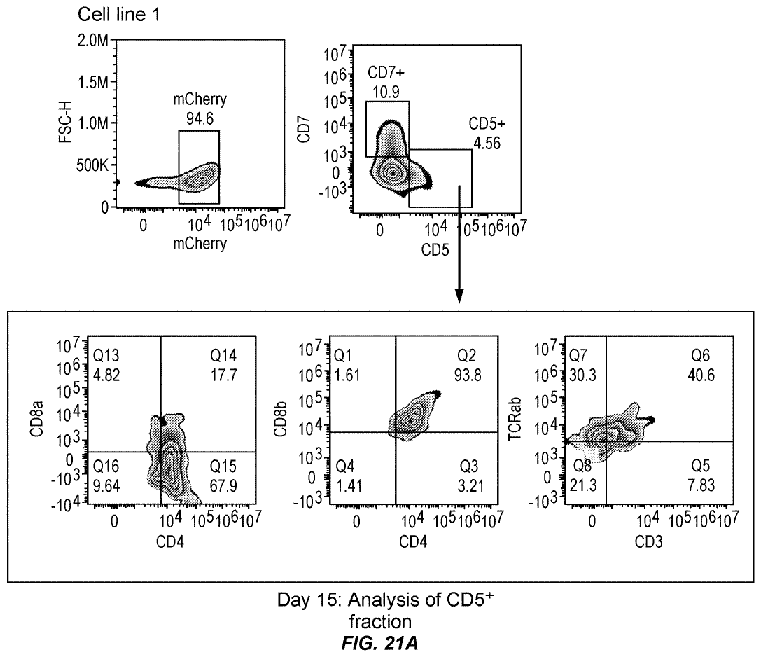

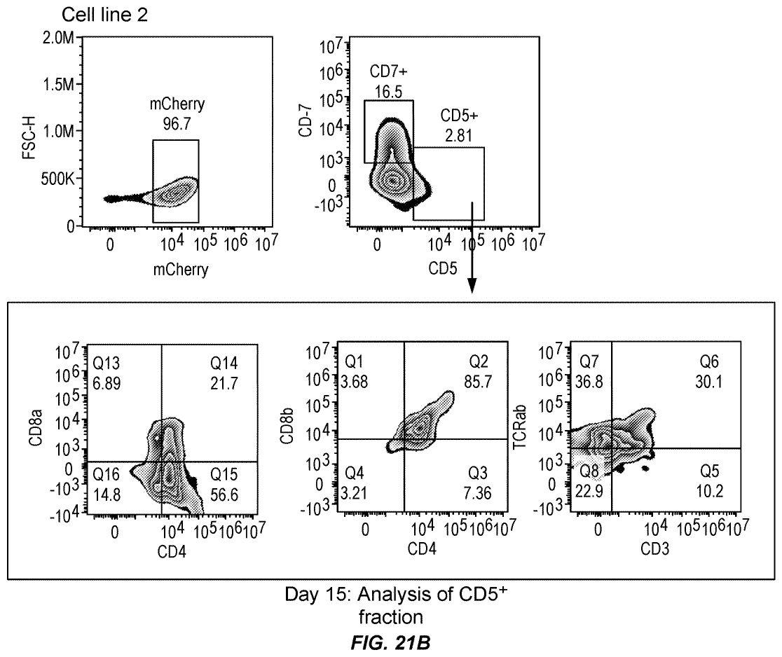

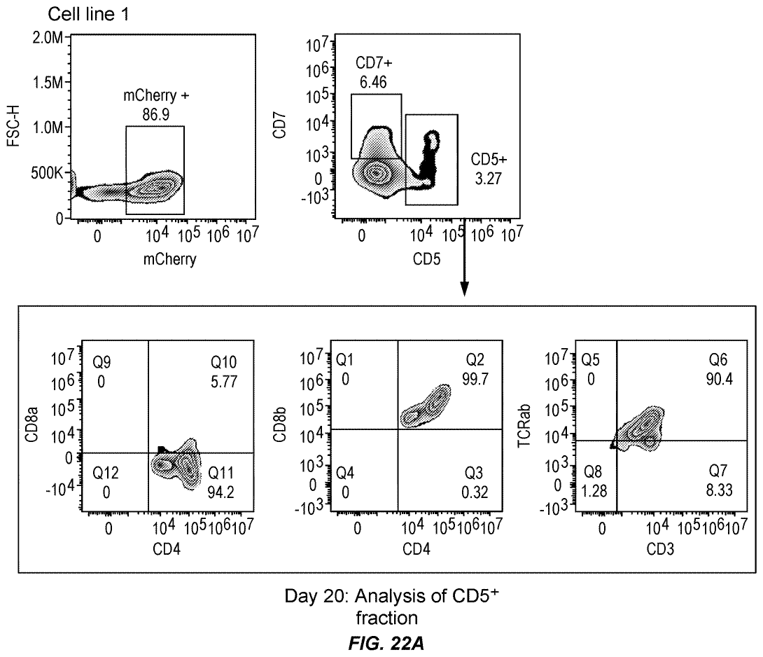

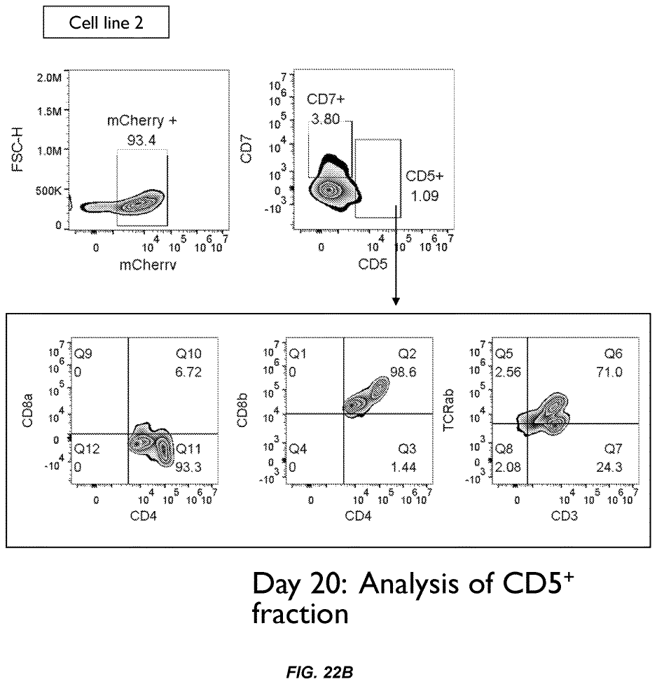

This example describes experiments conducted to generate CD4+ T cells from GAPDH-TCR engineered iPSCs s (Example 5) which were cultured with artificial antigen presenting substrates (aAPCs). The iPSCs were differentiated into CD34+ cells as described above. The CD34+ cells were plated onto aAPCs (i.e., APC4 of Example 6) and induced to differentiate into CD4+ T cells according to the differentiation scheme described above. At day 15 (FIGS. 21A and 21B) and day 20 (FIGS. 22A and 22B) cells were harvested and stained with antibodies against CD7, CD5, CD4, CD8 alpha, CD8 beta, CD3, and TCR. CD5 positive cells were gated and further evaluated for their expression of CD4, CD8 alpha, and CD8 beta.

FIGS. 21A and 21B shows an exemplary flow cytometry analysis of CD5+ cells from day 15 of T cell differentiation on an artificial antigen presenting substrate. The data show that, following 15 days of differentiation, 67.9% (cell line 1, FIG. 21A) or 56.6% (cell line 2, FIG. 21B) of CD5+ cells are CD4+ and CD8 alpha negative.

FIGS. 22A and 22B shows an exemplary flow cytometry analysis of CD5+ cells from day 20 of T cell differentiation on an artificial antigen presenting substrate. The data show that, following 20 days of differentiation, 94.2% (cell line 1, FIG. 22A) or 93.3% (cell line 2, FIG. 22B) of CD5+ cells are CD4+ and CD8 alpha negative.

Taken together, these data show that co-culture of iPSC derived progenitor T cells with aAPCs expressing a cognate antigen can result in the generation of a CD4+ cell.

### Example 8: Enhanced Differentiation of T Cell-Derived iPSCs into CD4+ T Cells

This example describes experimental work conducted to investigate the differentiation efficiency of iPSCs originating from T cells (T-iPSCs) in comparison to iPSCs derived from other cell types (non-T-iPSCs) when generating CD4+ T cells.

Briefly, one T-iPSC line and one non-T-iPSC line were differentiated into CD34+ cells using the method outlined in FIG. 3. These CD34+ cell lines were then further induced to differentiate into CD4+ T cells, as detailed in FIG. 4. The expansion capacity during T cell differentiation between cells derived from the T-iPSCs and non-T-iPSCs was then investigated.

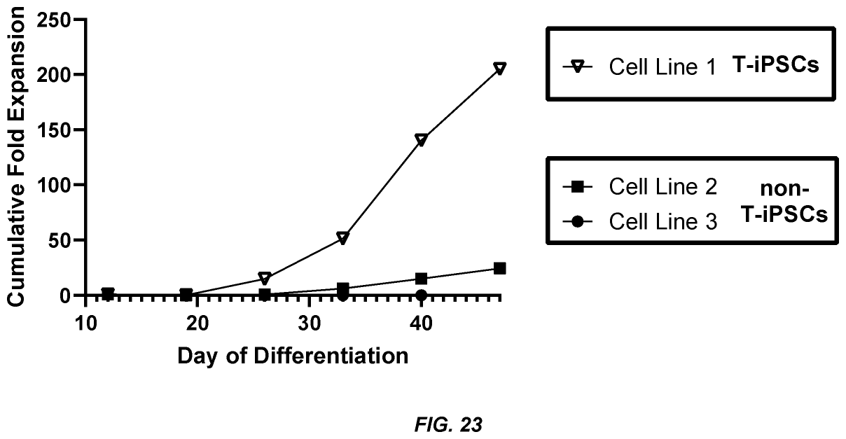

FIG. 23 show exemplary results from these experiments. These data showed that T-iPSCs had a higher expansion than non-T-iPSCs. This increased expansion is an indicator of the enhanced capacity for differentiation of T-iPSCs into CD4+ T cells. The higher expansion rate also suggests that T-iPSCs may maintain intrinsic properties conducive to T cell lineage commitment, likely due to their origin.

Furthermore, these data support the strategy of employing T-iPSCs for generating CD4+ T cells, particularly when enhanced through transgene overexpression, such as ThPOK.

### Example 9: Enhanced CD4+ T Cell Differentiation Via ThPOK Overexpression

This example describes experimental work conducted to investigate whether overexpressing ThPOK in progenitor T cells can facilitate the differentiation into CD4+ T cells.

Briefly, the two T-iPSC lines were differentiated into CD34+ cells using the method outlined in FIG. 3. These CD34+ cells were then further induced to differentiate into CD4+ T cells, as detailed in FIG. 4. On day 9, when a majority of the cells were DN (double-negative) cells, ThPOK was overexpressed in a subset of the culture dishes by the nucleofection with ThPOK-encoding mRNA. On day 10, cells treated with ThPOK mRNA, those nucleofected without mRNA (No mRNA), and cells not nucleofected (No EP) were collected, stained for various markers including CD45, CD4, CD8a, ThPOK, and Runx3, and analyzed via flow cytometry.

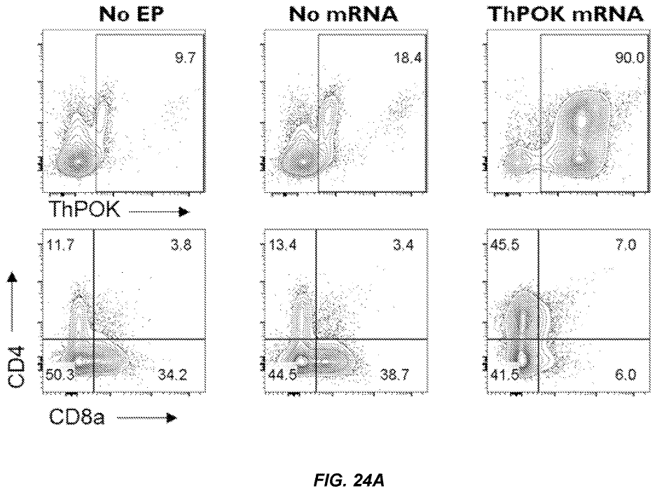

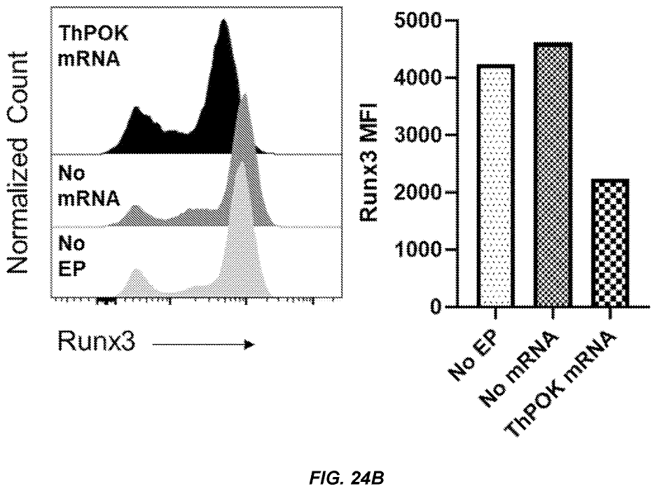

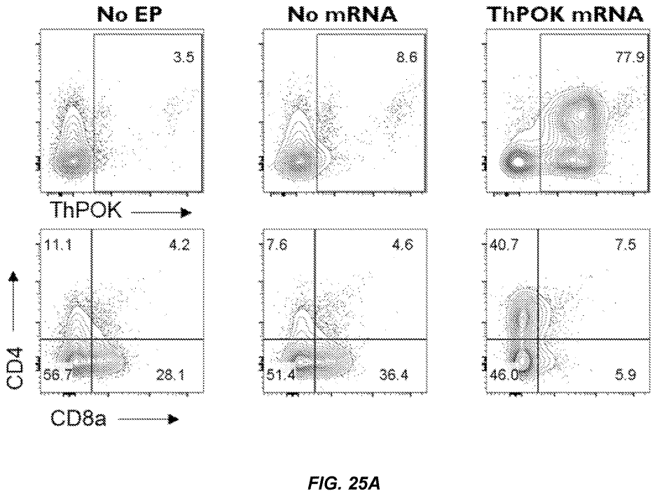

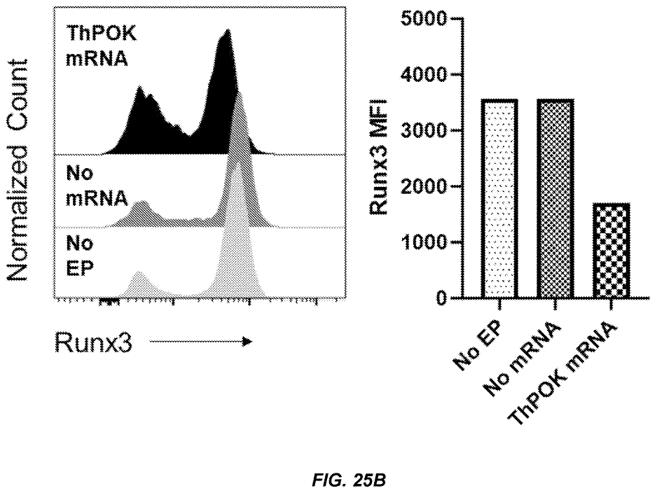

The results indicate that, after 10 days of differentiation, the ThPOK nucleofected cells exhibited high levels of ThPOK expression, with 90.0% in Cell Line 1 (FIG. 24A) and 77.9% in Cell Line 2 (FIG. 25A). Additionally, these cells showed significant CD4+CD8α-expression, accounting for 45.5% in Cell Line 1 (FIG. 24A) and 40.7% in Cell Line 2 (FIG. 25A). Notably, Runx3 expression, quantified by mean fluorescence intensity (MFI), was reduced in both cell lines, as illustrated in FIGS. 24B and 25B. Reduced Runx3 expression, in the context of overexpressed ThPOK, suggests a shift in T cell differentiation towards CD4+ T cells, as a reduction in Runx3 expression minimizes CD8+ T cell development pathways, enhancing the specificity and yield of the desired CD4+ T cell population.

### EQUIVALENTS AND SCOPE, INCORPORATION BY REFERENCE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members, are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. Thus, for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

What is claimed is:

1. An engineered progenitor T cell comprising:
a heterologous nucleic acid that, when activated, results in the expression of a lineage commitment factor at a level that is higher than that of a comparable wild-type cell to thereby facilitate differentiation towards a CD4+ T cell.

2. The engineered progenitor T cell of claim 1, wherein the engineered progenitor T cell comprises a CD4+CD8+ double positive (DP) phenotype.

3. The engineered progenitor T cell of claim 1, wherein the engineered progenitor T cell comprises a CD4−CD8− double negative (DN) phenotype.

4. The engineered progenitor T cell of any one of claims 1 to 3, wherein the heterologous nucleic acid is expressed by the engineered progenitor T cell.

5. The engineered progenitor T cell of any one of claims 1 to 4, wherein the heterologous nucleic acid encodes the lineage commitment factor.

6. The engineered progenitor T cell of any one of claim 1 to 5, wherein the lineage commitment factor comprises at least one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

7. The engineered progenitor T cell of any one of claims 1 to 6, wherein the lineage commitment factor comprises ThPOK.

8. The engineered progenitor T cell of any one of claims 1 to 7, wherein the heterologous nucleic acid is integrated in the genome of the engineered progenitor T cell in a sustained transcriptionally active payload region (STAPLR) or a sustained transgene expression locus (STEL).

9. The engineered progenitor T cell of any one of claims 1 to 8, wherein the heterologous nucleic acid is integrated into a STAPLR.

10. The engineered progenitor T cell of claim 9, wherein the STAPLR is selected from the group consisting of:
the intergenic region between the RPL34 gene and the OSTC gene;
the intergenic region between the ACTB gene and the FSCN1 gene;
the intergenic region between the AKIRIN1 gene and the NDUFS5 gene;
the intergenic region between the PRDX1 gene and the AKR1A1 gene;
the intergenic region between the PTGES3 gene and the NACA gene;
the intergenic region between the MLF2 gene and the PTMS gene;
the intergenic region between the RAB13 gene and the RPS27 gene;
the intergenic region between the JTB gene and the RAB13 gene;
the intergenic region between the AKR1A1 gene and the NASP gene;
the intergenic region between the NDUFS5 gene and the MACF1 gene;
the intergenic region between the SRSF9 gene and the DYNLL1 gene;
the intergenic region between the MYL6B gene and the MYL6 gene;
the intergenic region between the GPX1 gene and the RHOA gene;
the intergenic region between the HNRNPA2B1 gene and the CBX3 gene;
the intergenic region between the ROMO gene and the RBM39 gene; and
the intergenic region between the PA2G4 gene and the RPL41 gene.

11. The engineered progenitor T cell of claim 10, wherein the STAPLR is the intergenic region between the PRDX1 gene and the AKR1A1 gene.

12. The engineered progenitor T cell of any one of claims 1 to 11, wherein the heterologous nucleic acid is integrated at a location that is at least 100-5000 base pairs away from the nearest gene.

13. The engineered progenitor T cell of any one of claims 1 to 7, wherein the heterologous nucleic acid is integrated into the genome of the engineered progenitor T cell at or near a gene that is specifically expressed in a T cell.

14. The engineered progenitor T cell of any one of claims 1 to 7, wherein the heterologous nucleic acid is integrated in the genome of the engineered progenitor T cell at or near a TRAC locus.

15. The engineered progenitor T cell of any one of claims 1 to 14, wherein the heterologous nucleic acid further comprises an inducible promoter upstream of the lineage commitment factor.

16. The engineered progenitor T cell of any one of claims 1 to 15, wherein the heterologous nucleic acid, when activated, results in at least a 2-fold increase in expression of the lineage commitment factor as compared to a comparable wild-type cell under similar conditions and thereby steers differentiation of the engineered progenitor T cell into the CD4+ T cell.

17. The engineered progenitor T cell of any one of claims 1 to 16, wherein the engineered progenitor T cell further comprises a heterologous nucleic acid sequence encoding a second and optionally a third lineage commitment factor.

18. The engineered progenitor T cell of claim 17, wherein the second lineage commitment factor and optionally the third lineage commitment factor, when activated, promotes differentiation towards a CD4+ T cell or a CD4+ T cell subset.

19. The engineered progenitor T cell of claim 18, wherein the T cell subtype comprises a Treg cell.

20. The engineered progenitor T cell of any one of claims 1 to 19, wherein the engineered progenitor T cell is derived from an induced pluripotent stem cell.

21. The engineered progenitor T cell of claim 20, wherein the induced pluripotent stem cell is derived from a T cell comprising an autoantigen specific TCR.

22. The engineered progenitor T cell of any one of claims 1 to 21, wherein the engineered progenitor T cell is genetically modified to integrate an exogenous nucleic acid encoding a T cell receptor (TCR) or a chimeric antigen receptor (CAR).

23. The engineered progenitor T cell of claim 22, wherein the exogenous nucleic acid encodes the TCR and the TCR is specific to an autoantigen.

24. The engineered progenitor T cell of claim 23, wherein the exogenous nucleic acid is integrated within a STEL.

25. The engineered progenitor T cell of claim 24, wherein the STEL comprises a housekeeping gene.

26. The engineered progenitor T cell of claim 25, wherein the housekeeping gene comprises GAPDH.

27. The engineered progenitor T cell of claim 23, wherein the exogenous nucleic acid is integrated into a TCR alpha constant (TRAC) locus.

28. An engineered cell comprising:
a heterologous nucleic acid comprising a coding sequence for a lineage commitment factor that promotes differentiation of the engineered cell towards a CD4+ T cell, wherein the heterologous nucleic acid is integrated within the genome of the engineered cell in a sustained transcriptionally active payload region (STAPLR) or a sustained transgene expression locus (STEL).

29. The engineered cell of claim 28, wherein the heterologous nucleic acid is integrated at a location that is at least 100-5000 base pairs away from the nearest gene, and wherein the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

30. The engineered cell of claim 28 or 29, wherein the engineered cell is further modified to reduce the expression of a competing lineage commitment factor, and wherein the competing lineage commitment factor optionally comprises Runx3.

31. An engineered cell comprising a heterologous nucleic acid that, when activated, results in at least a 2-fold increase in expression of ThPOK as compared to a comparable wild-type cell under similar conditions, thereby promoting differentiation of the engineered cell towards a CD4+ T cell.

32. The engineered cell of claim 31, wherein the heterologous nucleic acid comprises a coding sequence for ThPOK.

33. The engineered cell of claim 31 or 32, wherein the heterologous nucleic acid further comprises an inducible promoter upstream of the coding sequence.

34. The engineered cell of any one of claims 31 to 33, wherein the engineered cell further comprises at least one heterologous coding sequence for a lineage commitment factor that, when activated, promotes differentiation of the CD4+ T cell subtype.

35. The engineered cell of claim 34, wherein the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, or c-Myb.

36. The engineered cell of claim 35, wherein the engineered cell comprises one of an embryonic stem cell, an induced pluripotent stem cell, a hematopoietic stem cell, a lymphoid progenitor cell, or a progenitor T cell.

37. The engineered cell of any one of claims 31 to 36, wherein the engineered cell is a stem cell that is engineered to express a TCR that recognizes an autoantigen upon differentiation into a T cell.

38. An artificial antigen presenting substrate (aAPS) comprising an immunomodulatory polypeptide.

39. The aAPS of claim 38, wherein the immunomodulatory polypeptide comprises an antigen presenting molecule.

40. The aAPS of claim 39, wherein the antigen presenting molecule is linked to an antigen by a peptide.

41. The aAPS of claim 39 or 40, wherein the antigen presenting molecule comprises a major histocompatibility complex (MHC) molecule.

42. The aAPS of claim 41, wherein the MHC molecule comprises an MHC-class II molecule.

43. The aAPS of claim 42, wherein the MHC-class II molecule comprises at least one of an HLA-DP molecule, an HLA-DM molecule, an HLA-DO molecule, an HLA-DQ molecule, or an HLA-DR molecule.

44. The aAPS of any one of claims 38 to 43, wherein the aAPS comprises an artificial antigen presenting cell (aAPC), a bead, a particle, or a nanoparticle.

45. The aAPS of claim 44, wherein the aAPS comprises the aAPC.

46. The aAPS of claim 45, wherein the aAPC comprises a cell that is immobilized to a solid surface.

47. The aAPS of any one of claims 44 to 46, wherein the aAPS further comprises at least one co-stimulatory molecule or a Notch ligand.

48. The aAPS of claim 47, wherein the aAPS comprises the at least one co-stimulatory molecule, and wherein the at least one co-stimulatory molecule comprises at least one of CD40, CD80, CD86, ICOS-L, CD58, or ICAM1.

49. The aAPS of claim 48, wherein the co-stimulatory molecule comprises CD80 or ICAM1.

50. The aAPS of claim 47, wherein the aAPS comprises the Notch ligand, and wherein the Notch ligand comprises DLL4.

51. The aAPS of any one of claims 38 to 50, wherein the aAPS is engineered to secrete one or more cytokines or growth factors.

52. The aAPS of claim 51, wherein the one or more cytokines or growth factors are selected to influence cell fate of a target cell into a CD4+ T cell or regulatory T cell.

53. The aAPS of claim 51 or 52, wherein the one or more cytokines or growth factors comprises TGF-b.

54. A composition comprising:
a. a stem cell, the engineered progenitor T cell of any one of claims 1 to 27, or the engineered cell of any one of claims 28-37 and
b. the aAPS of any one of claims 38 to 53.

55. A composition comprising the aAPS of any one of claims 38 to 53, and cell culture media.

56. The composition of claim 55, further comprising a stem or progenitor T cell that expresses, or is capable of expressing upon differentiation into a T cell, an antigen recognition receptor.

57. The composition of claim 56, wherein the antigen recognition receptor comprises a TCR.

58. The composition of claim 57, wherein the antigen of the aAPS is bound by the TCR of the stem or progenitor T cell.

59. A method of producing a CD4+ T cell, the method comprising:
combining a stem or progenitor T cell that expresses an antigen recognition receptor, or is capable of expressing said antigen recognition receptor upon differentiation into a T cell, with the aAPS of any one of claims 38 to 53;
differentiating the stem or progenitor T cell towards a CD4+ T cell; and
contacting the antigen presenting molecule of the aAPS with the antigen recognition receptor of the T cell thereby promoting differentiation of the stem or progenitor cell towards a CD4+ T cell.

60. The method of claim 59, wherein the combining comprises:
adhering the aAPS to a surface of a tissue culture dish; and
adding the stem or progenitor T cell to the tissue culture dish comprising the aAPS.

61. A method of making a CD4+ T cell, the method comprising:
activating a heterologous nucleic acid comprising a coding sequence for a lineage commitment factor within an engineered CD4−CD8− double negative (DN) cell or an engineered CD4+CD8+ double positive (DP) cell, wherein the activating results in an increased expression of said lineage commitment factor within the engineered DN cell or the engineered DP cell as compared to a comparable wild-type cell under similar conditions and thereby promotes the differentiation of the engineered DN cell or the engineered DP cell towards a CD4+ T cell.

62. The method of claim 61, wherein the activating occurs in the engineered DN cell.

63. The method of claim 61, wherein the activating occurs in the engineered DP cell.

64. The method of any one of claims 61 to 63, wherein the lineage commitment factor comprises one of CD4, CD25, FOXP3, CD45RA, CD62L, Helios, GITR, Ikaros, CTLA4, Gata3, Tox, ETS1, TCF7, LEF1, RORA, TNFR2, Eos, Irf5, SatB1, Gatal, c-Myb, or ThPOK.

65. The method of claim 64, wherein the lineage commitment factor comprises ThPOK.

66. The method of any one of claims 61 to 65, wherein the heterologous nucleic acid comprises an inducible promoter upstream of the lineage commitment factor.

67. The method of claim 66, wherein the activating comprises activating the inducible promoter upstream of the coding sequence.

68. The method of claim 62, wherein the activating comprises contacting the DN cell with doxycycline or a derivative thereof.

69. The method of claim 63, wherein the activating comprises contacting the DP cell with doxycycline or a derivative thereof.

70. The method of any one of claims 61 to 69, wherein the DN or DP cell is derived from an induced pluripotent stem cell.

71. The method of any one of claims 61 to 70, further comprising overexpressing the lineage commitment factor at least 2-fold greater within the engineered DN or DP cell as compared to the comparable wild-type cell under similar conditions.

72. The method of claim 62, wherein the DN cell comprises a DN3 or DN4 cell.

73. A method of obtaining a population of cells enriched for CD4+ T cells, the method comprising:
obtaining a CD4−CD8− (DN) cells or CD4+CD8+(DP) cells; and
overexpressing at least one exogenous lineage commitment factor within the DN cells or DP cells as compared to a comparable wild-type cell under similar conditions to thereby produce a population of cells enriched for CD4+ T cells.

74. The method of claim 73, wherein the method is performed with DN cells.

75. The method of claim 73, wherein the method is performed with DP cells.

76. The method of any one of claims 73 to 75, further comprising contacting the DN cells or DP cells with a first artificial antigen presenting substrate (aAPS).

77. The method of claim 76, wherein the method further comprises contacting the DN cells or the DP cells with a second aAPS, wherein the second aAPS is different than the first aAPS.

78. A method of treating a subject in need of immunosuppression, comprising administering to the subject a cell derived from the engineered progenitor T cell of any one of claims 1 to 27 or the engineered cell of any one of claims 28-37.

79. A method of treating cancer, comprising administering to a subject diagnosed with cancer a cell derived from the engineered progenitor T cell of any one of claims 1 to 27 or the engineered cell of any one of claims 28-37.

80. The method of claim 78 or claim 79, wherein the cell derived from the engineered cell is a Treg.

81. A method of treating a subject in need of immunosuppression, comprising administering to the subject a cell derived from the stem or progenitor T cell contained in the composition of any one of claims 56 to 58.

82. A method of treating a cancer, comprising administering to a subject diagnosed with cancer a cell derived from the stem or progenitor T cell contained in the composition of any one of claims 56 to 58.

83. The method of claim 81 or claim 82, wherein the cell derived from the stem or progenitor T cell is a Treg.

84. A method of treating a subject in need of immunosuppression, comprising administering to the subject a CD4+ T cell or a population of cells enriched for CD4+ T cells obtained from the method of any one of claims 59 to 77.

85. A method of treating a cancer, comprising administering to a subject diagnosed with cancer a CD4+ T cell or a population of cells enriched for CD4+ T cells obtained from the method of any one of claims 59 to 77.

86. The method of any one of claims 78, 80, 81, 83, 84, wherein the subject has an autoimmune disease or is a recipient of a transplant.

87. Use of the engineered progenitor T cell of any one of claims 1 to 27 or the engineered cell of any one of claims 28-37, in the manufacture of a medicament in treating a cancer or a subject in need of immunosuppression.

88. Use of the stem or progenitor T cell contained in the composition of any one of claims 56 to 58, in the manufacture of a medicament in treating cancer or a subject in need of immunosuppression.

89. Use of a CD4+ T cell or a population of cells enriched for CD4+ T cells obtained from the method of any one of claims 59 to 77, in the manufacture of a medicament in treating a cancer or a subject in need of immunosuppression.
